(19) United States
(12) Patent Application Publication
(10) Pub. No.: US 2007/0155720 A1
(43) Pub. Date: Jul. 5, 2007

(54) Co-administration of dopamine-receptor binding compounds

(75) Inventors: Prabhavathi B. Fernandes, Chapel Hill, NC (US); Richard Bernard Mailman, Chapel Hill, NC (US); David Earl Nichols, West Lafayette, IN (US); Robert Niel Postlethwait, Zionsville, IN (US)

Correspondence Address: BARNES & THORNBURG LLP, 11 SOUTH MERIDIAN, INDIANAPOLIS, IN, 46204, US

(73) Assignee: DarPharma, Inc., Chapel Hill, NC (US)

(21) Appl. No.: 10/584,019

(22) PCT Filed: Dec. 22, 2004
(86) PCT No.: PCT/US04/43145

§ 371 Date: Jun. 22, 2006

Related U.S. Application Data
(60) Provisional application No. 60/532,248, filed on Dec. 23, 2003.

(51) Int. Cl. A61K 31/55; A61K 31/4745; A61K 31/473

(52) U.S. Cl. 514/217.02; 514/291; 514/292; 514/298; 514/284

(57) ABSTRACT

Methods for treating a patient having neurological, psychotic, and psychiatric disorders are described comprising the steps of administering to the patient an effective amount of a partial and/or full dopamine D₁ receptor agonist, and administering to the patient an effective amount of a dopamine D₂ receptor antagonist. Pharmaceutical compositions comprising a dopamine D₁ receptor agonist and a dopamine D₂ receptor antagonist are also described. The D₁ dopamine receptor agonist and the D₂ dopamine receptor antagonist can be administered to the patient in the same or in a different composition or compositions.

### CROSS-REFERENCE TO RELATED APPLICATIONS

This application claims priority to U.S. provisional patent application Ser. No. 60/532,248 filed Dec. 23, 2003

### TECHNICAL FIELD

The invention relates to methods and compositions for treating patients having neurological, psychotic, and/or psychiatric disorders. More particularly, the invention relates to methods for treating patients having neurological, psychotic, and/or psychiatric disorders by co-administration of compounds having different dopamine receptor activities to the patient.

### BACKGROUND OF THE INVENTION

It is generally accepted that there are at least two pharmacological subtypes of dopamine receptors (the D₁ and D₂ receptor subtypes), each consisting of several molecular forms. D₁ receptors preferentially recognize the phenyltetrahydrobenzazepines and generally lead to stimulation of the enzyme adenylate cyclase, whereas D₂ receptors recognize the butyrophenones and benzamides and often are coupled negatively to adenylate cyclase, or are not coupled at all to this enzyme. It is now known that at least five dopamine receptor genes encode the D₁, D₂, D₃, D₄, and D₅ receptor isoforms or subtypes. The traditional classification of dopamine receptor subtypes, however, remains useful with the D₁-like class comprising the D₁ (D_{1A}) and the D₅ (D_{1B}) receptor subtypes, whereas the D₂-like class consists of the D₂, D_{2L}, D_{2S}, D₃, and D₄ receptor subtypes. Agonist stimulation of dopamine D₁ receptors is believed to activate adenylate cyclase to form cyclic AMP (cAMP), which in turn is followed by the phosphorylation of intracellular proteins. Agonist stimulation of D₂ dopamine receptors is believed to lead to decreased cAMP formation. Agonists at both subclasses of receptors are clinically useful. However, much work remains to fully understand the physiological events associated with the interaction of dopamine agonists with each of these receptor subtypes

Dopamine receptor agonists are of therapeutic interest for a variety of reasons. For example, it has been hypothesized that excessive stimulation of D₂ dopamine receptor subtypes may be linked to schizophrenia. Additionally, it is generally recognized that either excessive or insufficient dopaminergic activity in the central nervous system can cause hypertension, narcolepsy, and other behavioral, neurological, physiological, psychological, and movement disorders, including Parkinson's disease.

For example, schizophrenia is among the most common and the most debilitating of psychiatric diseases. Current estimates suggest a prevalence of schizophrenia at between 0.5 and 1% of the population.

Patients with schizophrenia and other neurological and psychiatric disorders, such as psychosis, bipolar disorder, anxiety states, and depression in combination with psychotic episodes, can have both “positive” symptoms, including delusions, hallucinations, impaired cognitive function, and agitation, as well as “negative” symptoms, including emotional unresponsiveness, impaired memory, and impaired cognitive function. Patients with these psychotic signs and symptoms can be treated with drugs that fall into the general classes of typical antipsychotic drugs and atypical antipsychotic drugs. The typical antipsychotic agents include phenothiazines, butyrophenones, and other non-phenothiazines such as loxapine and molindone. The atypical antipsychotic agents include the clozapine-like drugs, such as clozapine, olanzepine, quetiapine, ziprasidone, and the like, as well as several others, including risperidone, aripiprazole, and amisulpiride, among others. Whereas both of these typical and atypical antipsychotic agents are useful for treating the positive symptoms of the neurological disorders described herein, patients may not find total relief from the negative symptoms that may accompany these antipsychotic agents. In addition, recent studies suggest that the current antipsychotic therapy for treating positive symptoms of schizophrenia may in some cases exacerbate or facilitate the onset of such negative symptoms.

Dopamine agonists have also been developed to treat Parkinson's disease in an attempt to avoid some of the limitations of levodopa therapy, because levodopa therapy is not always a successful treatment, for example in certain late-stage disorders. In addition, by acting directly on postsynaptic dopamine receptors, selective dopamine agonists bypass the degenerating presynaptic neurons. Furthermore, these drugs do not rely on the same enzymatic conversion for activity required for levodopa, avoiding issues associated with declining levels of striatal dopa decarboxylase. In addition, agonists have the potential for longer half-lives than levodopa, and can also be designed to interact specifically with predetermined subpopulations of dopamine receptors.

However, it has been shown that administering a D₂ receptor antagonist down regulates D₁ receptors. Such down regulation was shown to have the overall effect of causing or increasing memory and cognition complications. Down regulation of D₁ and/or D₅ receptor mRNAs has been observed in the prefrontal and temporal cortices but not in the neostriatum of nonhuman primates after chronic treatment with certain antipsychotic medications.

In addition, numerous reports have been made that full D₁ agonists may cause D₁ receptor desensitization and even down regulation of dopamine D₁ receptor expression. Partial D₁ agonists may cause desensitization but generally do not cause down regulation of receptor expression. In addition, it has also been shown that short-term administration of a D₁ receptor agonist following the onset of memory or cognition complications arising from administering a D₂ receptor antagonist, alleviated the symptoms of such memory or cognition complications.

### SUMMARY OF THE INVENTION

The invention described herein generally pertains to compounds, compositions, and methods for treating neurological, psychotic, and/or psychiatric disorders by administering a plurality of such dopamine receptor active compounds or compositions.

The compounds useful in the methods and compositions described herein for treating neurological, psychotic, and/or psychiatric disorders include partial and/or full dopamine D₁ receptor agonists, and dopamine D₂ receptor antagonists. The partial and/or full D₁ receptor agonists, and D₂ receptor antagonists are co-administered either contemporaneously or simultaneously. In accordance with the methods and compositions described herein, an effective amount of a partial and/or full D₁ receptor agonist can be co-administered to a patient having a neurological disorder along with an effective amount of a D₂ receptor antagonist to reduce the symptoms of the neurological, psychotic, and/or psychiatric disorder. Illustratively, to reduce both the positive and the negative symptoms of disorders such as schizophrenia, a dopamine D₂ receptor antagonist is used to reduce the primary symptoms, and a dopamine D₁ receptor agonist is used to reduce the negative symptoms. The partial and/or full D₁ receptor agonist and the D₂ receptor antagonist can be administered to the patient having the neurological disorder either in the same or in a different composition or compositions. It is appreciated that simultaneous co-administration is facilitated by a unit or unitary dosage form that includes both the partial and/or full D₁ receptor agonists, and D₂ receptor antagonists.

As used herein, the term “D₁ receptor” refers to each and every D₁ and D₁-like receptor, alone or in various combinations, including the D₁ and D₅ receptors in humans, the D_{1A} and D_{1B} receptors found in rats, and other D₁-like receptors. Similarly, the term “D₂ receptor” refers to each and every D₂ and D₂-like receptor, alone or in various combinations, including the D₂, D_{2L}, D_{2S}, D₃, and D₄ receptors found in mammals.

In one illustrative embodiment, the dopamine agonist is a compound selected from the following group of compounds: 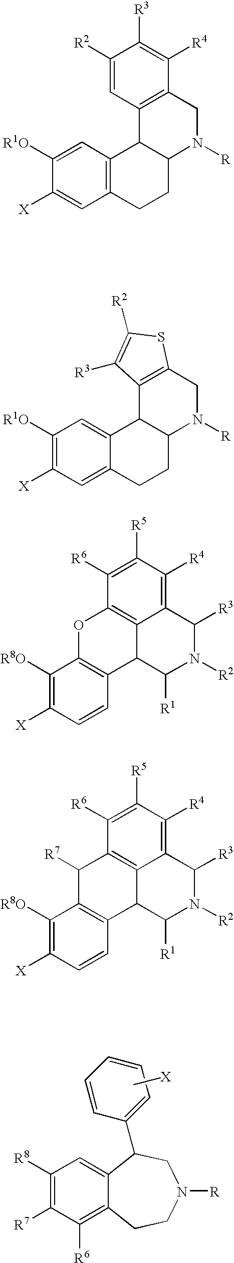
wherein, the groups R, R¹, R³, R⁴, R⁵, R⁶, R⁷, R⁸, and X are as defined herein.

It is appreciated that each of the foregoing compounds have one or more asymmetric carbon atoms or chiral centers, and that each may be prepared in or isolated in optically pure form, or in various mixtures of enantiomers or diastereomers. Each of the individual stereochemically pure isomers of the foregoing are contemplated herein. In addition, various mixtures of such stereochemically pure isomers are also contemplated, including but not limited to racemic mixtures that are formed from one pair of enantiomers.

In another illustrative aspect, the dopamine agonist is a compound selected from the following group of compounds: 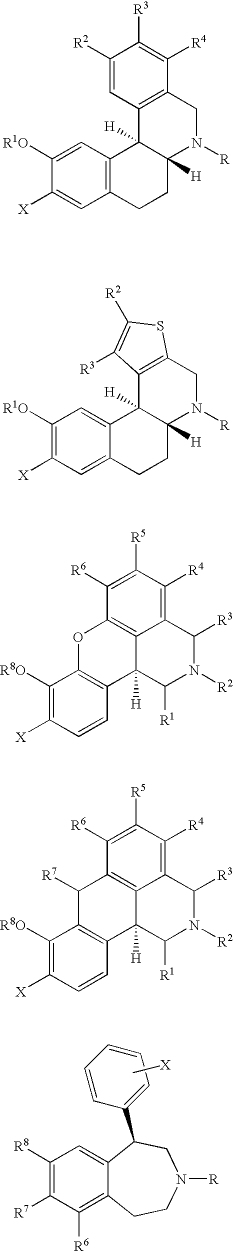
wherein, the groups R, R¹, R², R³, R⁴, R⁵, R⁶, R⁷, R⁸, and X are as defined herein, and the compounds are in optically pure form as shown, or are a racemic mixture with the relative stereochemistry shown.

In another embodiment, the dopamine D₂ receptor antagonist is an antipsychotic agent, and is illustratively selected from the typical and atypical families of antipsychotic agents. It is appreciated that atypical antipsychotics may generally be associated with less acute extrapyramidal symptoms, especially dystonias, and less frequent and smaller increases in serum prolactin concentrations associated with therapy. In one aspect, the typical antipsychotic agents include phenothiazines and non-phenothiazines such as loxapine, molindone, and the like. In another aspect, the atypical antipsychotic agents include the clozapine-like agents, and others, including aripiprazole, risperidone (3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one), amisulpiride, sertindole (1-[2-[4-[5-chloro-1-(4-fluorophenyl)-1H-indol-3-yl]-1-piperidinyl]ethyl]imidazolidin-2-one), and the like. Phenothiazines include, but are not limited to chlorpromazine, fluphenazine, mesoridazine, perphenazine, prochlorperazine, thioridazine, and trifluoperazine. Non-phenothiazines include, but are not limited to haloperidol, pimozide, and thiothixene. Other clozapine-like agents include, but are not limited to olanzapine (2-methyl-4-(4-methyl-1-piperazinyl)-10H-thieno[2,3-b][1,5]benzodiazepine), clozapine (8-chloro-11-(4-methyl-1-piperazinyl)-5H-dibenzo[b,e][1,4]diazepine), quetiapine (5-[2-(4-dibenzo[b,f][1,4]thiazepin-11-yl-1-piperazinyl)ethoxy]ethanol), ziprasidone (5-[2-[4-(1,2-benzoisothiazol-3-yl)-1-piperazinyl]ethyl]-6-chloro-1,3-dihydro-2H-indol-2-one), and the like. It is appreciated that other typical and atypical antipsychotic agents may be used in the methods and compositions described herein. It is also appreciated that various combinations of typical and atypical antipsychotic agents may be used in the methods and compositions described herein.

In another embodiment, a pharmaceutical composition is described. The composition includes a partial and/or full dopamine D₁ receptor agonist, a dopamine D₂ receptor antagonist, and a pharmaceutically carrier, excipient, diluent, or combination thereof. In one aspect, the D₁ receptor agonist is illustratively a compound selected from the group consisting of hexahydrobenzophenanthridines, hexahydrothienophenanthridines, phenylbenzodiazepines, chromenoisoquinolines, naphthoisoquinolines, and pharmaceutically acceptable salts thereof, including combinations of the foregoing. In another aspect, the pharmaceutical composition is a unit or unitary dosage form. It is to be understood that such unit or unitary dosage forms include kits or other formats that may require mixing prior to or immediately before administering to a patient.

In another illustrative embodiment, a method for treating a patient having a neurological, psychotic, and/or psychiatric disorder is described. The method comprises the steps of (a) administering to the patient an effective amount of a partial and/or full D₁ dopamine receptor agonist, and (b) administering to the patient an effective amount of a D₂ dopamine receptor antagonist. In one illustrative aspect, the dopamine agonist is a compound selected from the group consisting of hexahydrobenzophenanthridines, hexahydrothienophenanthridines, phenylbenzodiazepines, chromenoisoquinolines, naphthoisoquinolines, analogs and derivatives thereof, and pharmaceutically acceptable salts thereof, including combinations of the foregoing.

In another embodiment, methods are described wherein the D₁ dopamine receptor agonist and the D₂ dopamine receptor antagonist are administered to the patient in the same composition. In one variation, the D₁ dopamine receptor agonist and the D₂ dopamine receptor antagonist are administered to the patient in different compositions.

In another embodiment of the methods described herein, either or both of the D₁ receptor agonist and/or the D₂ receptor antagonist are administered intermittently or discontinuously. In one aspect, the D₂ receptor agonist is administered continuously or more regularly than the D₁ receptor agonist. In another aspect, the D₁ receptor agonist is administered in a discontinues or intermittent manner such that a first dose is administered but is allowed to decrease through the intervention or biological, metabolism, excretion, enzymatic, chemical, or other process to achieve a second lower dose, where the second lower dose is a suboptimal dose sufficiently incapable of agonizing the D₁ dopamine receptor to a full extent. In another aspect, the D₁ receptor agonist is a compound that has a half-life of less than about six hours.

### BRIEF DESCRIPTION OF THE DRAWINGS

FIG. 1 illustrates the chemical conversions detailed in Examples 1-5 for preparation of dihydrexidine and other hexahydrobenzo[a]phenanthridine compounds: (a) 1. Benzylamine, H₂O; 2. ArCOCl, Et₃N; (b) hv; (c) BH₃-THF; (d) H₂, 10% Pd/C; (e) 48% HBr, reflux.

FIG. 2 illustrates the chemical conversions detailed in Examples 6-8 for preparation of dinoxyline and other chromeno[4,3,2-de]isoquinoline compounds: (a) 1. NaH, THF; 2. CH₃OCH₂Cl, 0° C. to r.t.; 82%; (b) 1. n-BuLi; 2. −78° C. to r.t.; 76%; (c) KNO₃, H₂SO₄; 89%; (d) Pd(Ph₃)₄, KOH, Bu₄N⁺Cl⁻, H₂O, DME, reflux; (e) TsOH.H₂O, MeOH; 98%; (f) DMF, K₂CO₃, 80° C.; 86%; (g) PtO₂, AcOH, HCl, H₂; 99%; (h) R-L, K₂CO₃, acetone; (i) BBr₃, CH₂Cl₂, −78° C. to r.t.; 72%.

FIG. 3 illustrates the chemical conversions detailed in Example 9 for preparation of 2-methyl-2,3-dihydro-4(1H)-isoquinolone, an illustrative intermediate in the synthesis of dinapsoline and other naphthoisoquinolines, from ethyl 2-toluate: (a) NBS (N-bromosuccinimide, benzoylperoxide, CCl₄, reflux; (b) sarcosine ethylester HCl, K₂CO₃, acetone; (c) 1. NaOEt, EtOH, reflux, 2. HCl, reflux.

FIG. 4 illustrates the chemical conversions detailed in Example 10 for preparation of dinapsoline and other naphthoisoquinolines from substituted benzamides, as illustrated by 2,3-dimethoxy-N,N′-diethylbenzamides: (a) 1. sec-butyllithium, TMEDA, Et₂O, −78° C., 2. Compound 20, 3. TsOH, toluene, reflux; (b) 1. 1-chloroethylchloroformate, (CH₂Cl)₂, 2. CH₃OH; (c) TsCl, Et₃N; (d) H₂, Pd/C, HOAc; (e) BH₃ THF; (f) conc. H₂SO₄, −40° C. to −5° C.; (g) Na/Hg, CH₃OH, Na₂HPO₄; (h)BBr₃, CH₂Cl₂.

FIG. 5 illustrates an alternate synthesis for preparation of dinapsoline and other naphthoisoquinolines from substituted benzenes and isoquinolines, as illustrated by 1-bromo-3,4-methylenedioxybenzene, which may also be used to prepare optically active compounds: (a) Br₂/AlCl₃/neat; (b) 1. n-BuLi, 2. DMF; (c) LDA; (d) add 32 to 33; (e) NaBH₃CN in HC1/THF; (f) BBr₃/CH₂Cl₂.

### DETAILED DESCRIPTION

The compounds, compositions, and methods described herein are useful for co-administration of dopamine receptor-binding compounds including partial and/or full dopamine D₁ receptor agonists and dopamine D₂ receptor antagonists. The dopamine D₁ receptor agonists may have biological activities ranging from compounds with selective D₁ receptor agonist activity to compounds with potent activities affecting both D₁ and D₂ dopamine receptors and various subtypes thereof. In accordance with the methods and compositions described herein, an effective amount of a partial and/or full D₁ receptor agonist can be co-administered to a patient having a neurological disorder along with an effective amount of a D₂ receptor antagonist to reduce the symptoms of the neurological disorder (e.g., to reduce both the positive and the negative symptoms of neurological disorders such as schizophrenia). The partial and/or full D₁ receptor agonist and the D₂ receptor antagonist can be administered to the patient having the neurological disorder either in the same or in a different composition or compositions.

It is appreciated that in certain variations of the compounds, compositions, and methods described herein, full dopamine D₁ agonists are included and partial dopamine D₁ agonists are excluded. For certain diseases states, or disease stages, partial dopamine D₁ agonists may not be as effective as full dopamine D₁ agonists. Illustrative of this variation, compounds of formulae I-IV are used in the compounds, compositions, and methods described herein, and in particular those examples of formulae I-IV that are full dopamine D₁ receptor agonists.

Exemplary neurological disorders that can be treated with the method and composition described herein include such neurological disorders as schizophrenia, schizophreniform disorder, schizoaffective disorders, including those characterized by the occurrence of a depressive episode during the period of illness, bipolar disorder, depression in combination with psychotic episodes, and other disorders that include a psychosis. The types of schizophrenia that may be treated include Paranoid Type Schizophrenia, Disorganized Type Schizophrenia, Catatonic Type Schizophrenia, Undifferentiated Type Schizophrenia, Residual Type Schizophrenia, Schizophreniform Disorder, Schizoaffective Disorder, Schizoaffective Disorder of the Depressive Type, and Major Depressive Disorder with Psychotic Features. Typically, the neurological disorders that can be treated have both “positive” symptoms (e.g., delusions, hallucinations, impaired cognitive function, and agitation) and “negative” symptoms (e.g., emotional unresponsiveness).

It is to be understood that various forms of schizophrenia may be treatable using the methods and compositions described herein. It is also appreciated that psychotic conditions as described herein include schizophrenia, schizophreniform diseases, acute mania, schizoaffective disorders, and depression with psychotic features. The titles given these conditions may represent multiple disease states. Illustratively, the disease state may be references by the classification in the Diagnostic and Statistical Manual of Mental Disorders, 4th Edition, published by the American Psychiatric Association (DSM). The DSM code numbers for several disease states include Paranoid Type Schizophrenia 295.30, Disorganized Type Schizophrenia 295.10, Catatonic Type Schizophrenia 295.20, Undifferentiated Type Schizophrenia 295.90, Residual Type Schizophrenia 295.60, Schizophreniform Disorder 295.40, Schizoaffective Disorder 295.70, Schizoaffective Disorder of the Depressive Type and Major Depressive Disorder with Psychotic Features 296.24, 296.34. It is also understood that psychoses are often associated with other diseases and conditions, or caused by such other conditions, including with neurological conditions, endocrine conditions, metabolic conditions, fluid or electrolyte imbalances, hepatic or renal diseases, and autoimmune disorders with central nervous system involvement, and with use or abuse of certain substances, including but not limited to cocaine, methylphenidate, dexmethasone, amphetamine and related substances, cannabis, hallucinogens, inhalants, opioids, phencyclidine, sedatives, hypnotics, and anxiolytics. Psychotic disorders may also occur in association with withdrawal from certain substances. These substances include, but are not limited to, sedatives, hypnotics and anxiolytics. Another disease state treatable with the methods and compositions described herein includes schizotypal personality disorder, a schizophrenia spectrum disorder that is related genetically, phenomenology, and neurobiology, and pharmacologically to chronic schizophrenia, and shares many of the cognitive deficits of schizophrenia, although typically to a lesser degree of severity.

Other disorders that have a psychotic component and a depressive component that can be treated include premenstrual syndrome, anorexia nervosa, substance abuse, head injury, and mental retardation. Additionally, endocrine conditions, metabolic conditions, fluid or electrolyte imbalances, hepatic or renal diseases, and autoimmune disorders with central nervous system involvement which have a psychotic component and a depressive component may be treated with the composition and method described herein.

It is surprisingly found that administering a D₁ receptor agonist contemporaneously or simultaneously with a D₂ receptor antagonist may alleviate or cure, or slow or prevent the onset of, symptoms associated with neurological, psychiatric, and/or psychotic disease states. In one aspect, the symptoms include memory loss, memory disorders, cognitive disorders, and dementia.

In particular, it is appreciated that administering a D₁ agonist contemporaneously or simultaneously with a D₂ antagonist may avoid the onset of symptoms associated with administering the D₂ antagonist in treatment alone, including avoiding the onset of memory and/or cognition complications. It is further appreciated that although a rescue treatment that includes treatment with a dopamine D₁ receptor agonist following the onset of negative symptoms associated with treatment involving a D₂ antagonist alone also may be effective, in some aspects such cycling of D₁ receptor activity with the accompanying onset of symptoms may be less desirable than avoiding the symptoms at the outset, which may be advantageous or more desirable. It is further appreciated that in some aspects such cycling may also erode the maximum recovery that may be achieved with such rescue treatment protocols, making less likely the recovery to original levels, as measured by D₁ activity or evaluations of memory and/or cognition.

It is further appreciated that methods of treating patients suffering from or susceptible to suffering from disease states that may respond to treatment according to the methods described herein a long-term protocol are easier to administer and/or monitor when using the simultaneous or contemporaneous treatment protocols described herein. Such simultaneous or contemporaneous treatment protocols may remove the need to measure or evaluate negative side effects from D₂ receptor antagonist treatment to decide upon the timing for initiation of a subsequent rescue treatment to alleviate such side effects by treating with a D₁ receptor agonist. Illustrative disease states that may benefit from the simultaneous or contemporaneous treatment protocols described herein include, but are not limited to, schizophrenia, dementia, senile dementia, presenile dementia, bipolar disorder, Alzheimer's disease (AD), Parkinson's disease (PD), psychosis, acute mania, mild anxiety states, depression, including depression in combination with psychotic episodes, memory loss, cognition loss and dysfunction, attention deficit hyperactivity disorder (ADHD), attention deficit disorder (ADD), drug or substance abuse, sexual dysfunction, autism, other neurodegenerative diseases, and other disease states that may arise from dysregulation or dysfunction of dopamine activity in the central nervous system (CNS).

It is further appreciated that interneuron acetylcholine esterase release may exacerbate the memory and cognition complications associated with D2 antagonist treatment, especially when such release occurs in the frontal cortex and other area of the brains associated with cognitions and memory. It has been shown that lower acetylcholine levels may the cause of or may exacerbate cognition and memory problems.

In another illustrative embodiment, the partial and/or full D₁ dopamine receptor agonist can be selective for a dopamine D₁ receptor subtype, such as the D₁ or D₅ receptor subtype in humans, or the D_{1A} or D_{1B} receptor subtype in rodents, and like receptor subtypes. In another embodiment, the partial and/or full D₁ dopamine receptor agonist can exhibit activity at both the D₁ and D₂ dopamine receptor subtypes. For example, the full D₁ dopamine receptor agonist can be about equally selective for the D₁ and D₂ dopamine receptor subtypes, or can be more active at the D₁ compared to the D₂ dopamine receptor subtypes. In another embodiment, the partial and/or full D₁ dopamine receptor agonist can be selective for a D₁ dopamine receptor or receptor subtype associated with a particular tissue. In another embodiment, the partial and/or full D₁ dopamine receptor agonist can be selective for a D₁ dopamine receptor or receptor subtype capable of exhibiting functional selectivity with the D₁ dopamine receptor agonist.

It is to be further understood that references to receptor selectivity include functional selectivity at dopamine receptors. Such functional selectivity may further distinguish the activity of the compounds and compositions described herein to allow the treatment of more specifically predetermined symptoms. For example, compounds and compositions that are selective for a particular dopamine receptor, illustratively the D₁ receptor, may yet exhibit a second layer of selectivity where such compounds and compositions show functional activity at dopamine D₁ receptors in one or more tissues, but not in other tissues. Illustrative of such functional selectivity is the reported selectivity of dihydrexidine for postsynaptic neurons over presynaptic neurons. Other functional selectivity is contemplated herein.

For example, dihydrexidine, (±)-trans-10,11-dihydroxy-5,6,6a,7,8,12b-hexahydrobenzo[a]phenanthridine hydrochloride, has been reported to have nanomolar affinity and about 12-fold to about 60-fold selectivity for the D₁ over the D₂ receptor (2.2 nM and 183 nM, respectively). Phamacokinetic studies in rodents and non-human primates have shown that significant blood levels can be measured following intravenous (iv), subcutaneous (sc), and oral (po) administration. These studies also show that this drug is cleared rapidly from plasma. However, the pharmacodynamic studies demonstrate a much longer duration of action exhibited with the sc route of administration, than might be expected from the plasma half-life of dihydrexidine.

The compounds, compositions, and methods described herein may be evaluated by using conventional animal models for cognition, such as for routine optimization of dosages, dosage forms, and the like. Illustratively, animal models include evaluation of reference memory in a radial arm maze (Packard et al., J. Neurosci. 9:1465-72 (1989)); Packard and White, Behav. Neural. Biol. 53:39-50 (1990)); Colombo et al., Behav. Neurosci. 103:1242-1250 (1989)), active (Kirby & Polgar, Physiol. Psychol. 2:301-306 (1974)) and passive avoidance (Packard & White, Behav. Neurosci. 105:295-306 (1991)); Polgar et al., Physiol. Psychol. 9:354-58 (1981)), delayed response performance (Arnsten et al., Psychopharmacol. 116:143-51 (1994)), Morris water maze (Wishaw et al., Behav. Brain Res. 24:125-138 (1987)) and split-T maze (Colombo et al. (1989)). It is appreciated that lesions of the nigrostriatal tract with 6-hydroxydopamine (6-OHDA) impair a variety of learning tasks including avoidance conditioning (Neill et al., Pharmacol. Biochem. Behav. 2:97-103 (1974)) and Morris water maze (Wishaw & Dunnett, Behav. Brain. Res. 18:11-29 (1985); Archer et al., Pharmacol. Biochem. Behav. 31:357-64 (1988)), each of which may be used to evaluate the compounds, compositions, and methods described herein. The disclosures of each of the foregoing are incorporated herein by reference.

In one illustrative embodiment, the dopamine agonist is a compound selected from the group consisting of hexahydrobenzophenanthridines, hexahydrothienophenanthridines, phenylbenzodiazepines, chromenoisoquinolines, naphthoisoquinolines, analogs and derivatives thereof, and pharmaceutically acceptable salts thereof, including combinations of the foregoing.

In another illustrative aspect, the dopamine agonist is a compound selected from the following group of compounds: 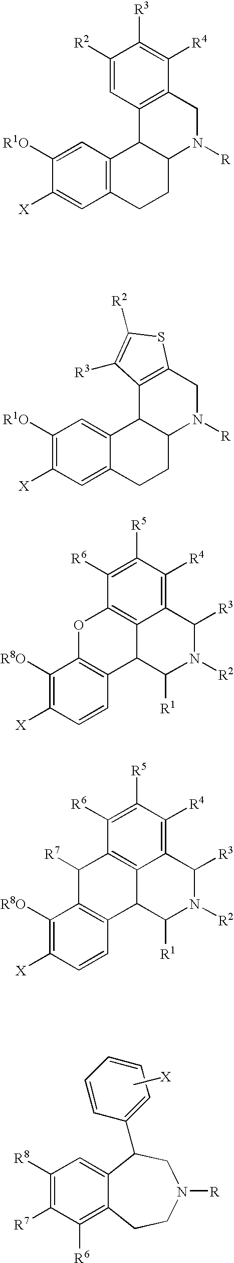
wherein, the groups R, R¹, R², R³, R⁴, R⁵, R⁶, R⁷, R⁸, and X are as defined herein.

It is appreciated that each of the foregoing compounds have one or more asymmetric carbon atoms or chiral centers, and that each may be prepared in or isolated in optically pure form, or in various mixtures of enantiomers or diastereomers. Each of the individual stereochemically pure isomers of the foregoing are contemplated herein. In addition, various mixtures of such stereochemically pure isomers are also contemplated, including but not limited to racemic mixtures that are formed from one pair of enantiomers. In another illustrative aspect, the dopamine agonist is a compound selected from the following group of compounds: 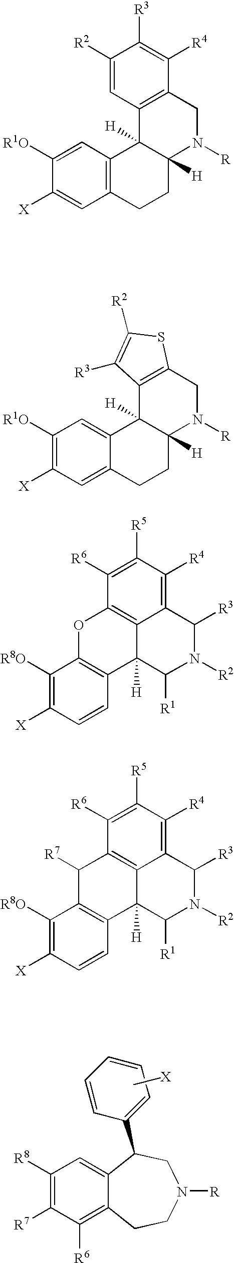
wherein, the groups R, R¹, R², R³, R⁴, R⁵, R⁶, R⁷, R⁸, and X are as defined herein, and the compounds are in optically pure form as shown, or are a racemic mixture with the relative stereochemistry shown.

In one embodiment, the D₁ dopamine receptor agonist is a hexahydrobenzo[a]phenanthridine compound. Exemplary hexahydrobenzo[a]phenanthridine compounds for use in the method and composition described herein include, but are not limited to, trans-5,6,6a,7,8,12b-hexahydrobenzo[a]phenanthridine compounds of Formula I: 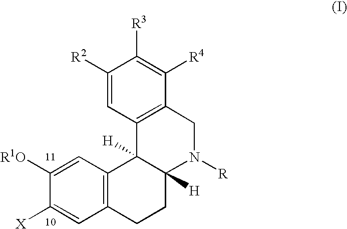
and pharmaceutically acceptable salts thereof, wherein R is hydrogen or C₁-C₄ alkyl; R¹ is hydrogen, acyl, such as C₁-C₄ alkanoyl, benzoyl, pivaloyl, and the like, or an optionally substituted phenyl or phenoxy protecting group, such as a prodrug and the like; X is hydrogen, fluoro, chloro, bromo, iodo or a group of the formula —OR⁵ wherein R⁵ is hydrogen, C₁-C₄ alkyl, acyl, such as C₁-C₄ alkanoyl, benzoyl, pivaloyl, and the like, or an optionally substituted phenyl or phenoxy protecting group, provided that when X is a group of the formula —OR⁵, the groups R¹ and R⁵ can optionally be taken together to form a —CH₂— or —(CH₂)₂— group, thus representing a methylenedioxy or ethylenedioxy functional group bridging the C-10 and C-11 positions on the hexahydrobenzo[a]phenanthridine ring system; and R², R³, and R⁴ are each independently selected from hydrogen, C₁-C₄ alkyl, phenyl, fluoro, chloro, bromo, iodo, and a group —OR⁶ wherein R⁶ is hydrogen, acyl, such as C₁-C₄ alkanoyl, benzoyl, pivaloyl, and the like, or an optionally substituted phenyl or pehnoxy protecting group; and pharmaceutically acceptable salts thereof. It is appreciated that compounds having Formula I are chiral.

As used herein, the term “acyl” refers to an optionally substituted alkyl or aryl radical connected through a carbonyl (C═O) group, such as optionally substituted alkanoyl, and optionally substituted aroyl or aryloyl. Illustrative acyl groups include, but are not limited to C₁-C₄ alkanoyl, acetyl, propionyl, butyryl, pivaloyl, valeryl, tolyl, trifluoroacetyl, anisyl, and the like.

In another embodiment, when X in Formula I is a group of the formula —OR⁵ the groups R¹ and R⁵ can be taken together to form a —CH₂— or —(CH₂)₂— group, thus representing a methylenedioxy or ethylenedioxy functional group bridging the C-10 and C-11 positions on the hexahydrobenzo[a]phenanthridine ring system.

In another embodiment, at least one of R², R³, and R⁴ is other than hydrogen. It is appreciated that the phenoxy protecting groups used herein may diminish or block the reactivity of the nitrogen to which they are attached. In addition, the phenoxy protecting groups used herein may also serve as prodrugs, and the like. It is understood that the compounds of Formula I are chiral. It is further understood that although a single enantiomer is depicted, each enantiomer, or various mixtures of each enatiomer are contemplated as included in the methods, and compositions described herein.

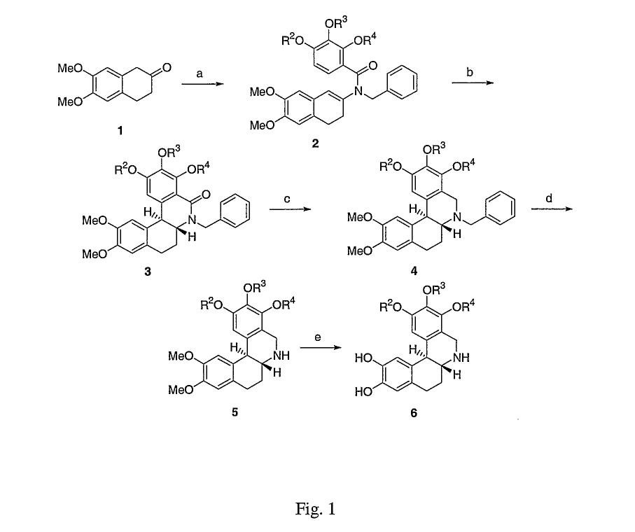

In accordance with the method and composition described herein, “C₁-C₄ alkoxy” as used herein refers to branched or straight chain alkyl groups comprising one to four carbon atoms bonded through an oxygen atom, including, but not limited to, methoxy, ethoxy, and t-butoxy. The compounds of Formula I are prepared using the same preparative chemical steps described for the preparation of the hexahydrobenzo[a]phenanthridine compounds (see FIG. 1) using the appropriately substituted benzoic acid acylating agent starting material instead of the benzoyl chloride reagent used in the initial reaction step. Thus, for example, the use of 4-methylbenzoyl chloride will yield a 2-methyl-hexahydrobenzo[a]phenanthridine compound.

In another embodiment of compounds of formula I, where X is —OR⁵, R¹ and R⁵ are different. In one aspect, one of R¹ and R⁵ is hydrogen or acetyl and the other of R¹ and R⁵ is selected from the group consisting of (C₃-C₂₀)alkanoyl, halo-(C₃-C₂₀)alkanoyl, (C₃-C₂₀)alkenoyl, (C₄-C₇)cycloalkanoyl, (C₃-C₆)-cycloalkyl(C₂-C₁₆)alkanoyl, aroyl which is unsubstituted or substituted by 1 to 3 substituents selected from the group consisting of halogen, cyano, trifluoromethanesulphonyloxy, (C₁-C₃)alkyl and (C₁-C₃)alkoxy, which latter may in turn be substituted by 1 to 3 halogen atoms, aryl(C₂-C₁₆)alkanoyl which is unsubstituted or substituted in the aryl moiety by 1 to 3 substituents selected from the group consisting of halogen, (C₁-C₃)alkyl and (C₁-C₃)alkoxy, which latter may in turn be substituted by 1 to 3 halogen atoms: and hetero-arylalkanoyl having one to three heteroatoms selected from O, S and N in the heteroaryl moiety and 2 to 10 carbon atoms in the alkanoyl moiety and which is unsubstituted or substituted in the heteroaryl moiety by 1 to 3 substituents selected from the group consisting of halogen, cyano, trifluoromethanesulphonyloxy, (C₁-C₃)alkyl, and (C₁-C₃)alkoxy, which latter may in turn be substituted by 1 to 3 halogen atoms, and the physiologically acceptable salts thereof.

In another embodiment, the D₁ dopamine receptor agonist for use in the method and composition described herein is represented by compounds having Formula II: 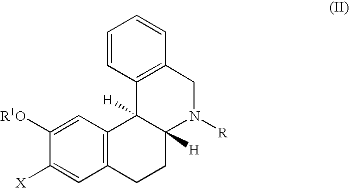
wherein R, R¹, and X are as defined in Formula I, and pharmaceutically acceptable salts thereof. It is appreciated that compounds having Formula II are chiral. It is further appreciated that although a single enantiomer is depicted, each enantiomer alone and/or various mixtures, including racemic mixtures, of each enantiomer are contemplated, and may be included in the compounds, compositions, and methods described herein.

The term “C₁-C₄ alkyl” as used herein refers to straight-chain or branched alkyl groups comprising one to four carbon atoms, such as methyl, ethyl, propyl, isopropyl, butyl, isobutyl, sec-butyl, cyclopropylmethyl, and the like. The selectivity of the compounds for the dopamine D₁ and D₂ receptors may be affected by the nature of the nitrogen substituent. Optimal dopamine D₁ agonist activity has been noted where R in formulae I-II is hydrogen or methyl. One compound of Formula II for use in the method and composition of the present invention is trans-10,11-dihydroxy-5,6,6a,7,8,12b-hexahydrobenzo[a]phenanthridine hydrochloride, denominated hereinafter as “dihydrexidine.”

N-Alkylation may be used to prepare compounds of formula I-II wherein R is other than hydrogen, and can be effected using a variety of known synthetic methods, including, but not limited to, reductive animation of the compounds wherein R═H with an aldehyde and a reducing agent, treatment of the same with an alkyl halide, treatment with a carboxylic acid in the presence of sodium borohydride, or treatment with carboxylic acid anhydrides followed by reduction, for example with lithium aluminum hydride or with borane as the reducing agent.

All active compounds described herein bear an oxygen atom at the C-11 position as shown in formulae I-II above. The C-10 unsubstituted, C-11 hydroxy compounds possess dopamine D₁ antagonist, or weak agonist activity, depending on the alkyl group that is attached to the nitrogen atom. The more potent dop amine D₁ agonist compounds exemplified herein have a 10,11-dioxy substitution pattern, in particular, the 10,11-dihydroxy substituents. However, the 10,11-dioxy substituents need not be in the form of hydroxyl groups. Masked hydroxyl groups, or prodrug (hydroxyl protecting) groups can also be used. For example, esterification of the 10,11-hydroxyl groups with, for example, benzoic acid or pivalic acid ester forming compounds (e.g., acid anhydrides) yields 10,11-dibenzoyl or dipivaloyl esters that are useful as prodrugs, i.e., they will be hydrolyzed in vivo to produce the biologically active 10,11-dihydroxy compound. A variety of biologically acceptable carboxylic acids can also be used. Furthermore, the 10,11-dioxy ring substitution can be in the form of a 10,11-methylenedioxy or ethylenedioxy group. In vivo, body metabolism will cleave this linkage to provide the more active 10,11-dihydroxy functionality. Compound potency and receptor selectivity can also be affected by the nature of the nitrogen substituent.

In another embodiment of the method and composition described herein, C₂, C₃, and/or C₄-substituted trans-5,6,6a,7,8,12b-hexahydrobenzo[a]phenanthridines can be used as the D₁ dopamine receptor agonist. The selectivity of these compounds for dopamine receptor subtypes varies, depending on the nature and positioning of substituent groups. Substitution at the C₂, C₃, and/or C₄ position on the benzophenanthridine ring system controls affinity for the dopamine receptor subtypes and concomitantly receptor selectivity. Thus, for example, 2-methyldihydrexidine has D₁ potency and efficacy comparable to dihydrexidine, while it has a five-fold enhanced selectivity for the D₁ receptor. In contrast, the compound 3-methyldihydrexidine, although retaining D₁ potency and efficacy comparable to dihydrexidine, has greater D₂ potency, making it less selective but better able to activate both types of receptors.

In another embodiment of compounds of formula II, where X is —OR⁵, R¹ and R⁵ are different. In one aspect, one of R¹ and R⁵ is hydrogen or acetyl and the other of R¹ and R⁵ is selected from the group consisting of (C₃-C₂₀)alkanoyl, halo-(C₃-C₂₀)alkanoyl, (C₃-C₂₀)alkenoyl, (C₄-C₇)cycloalkanoyl, (C₃-C₆)-cycloalkyl(C₂-C₁₆)alkanoyl, aroyl which is unsubstituted or substituted by 1 to 3 substituents selected from the group consisting of halogen, cyano, trifluoromethanesulphonyloxy, (C₁-C₃)alkyl and (C₁-C₃)alkoxy, which latter may in turn be substituted by 1 to 3 halogen atoms, aryl(C₂-C₁₆)alkanoyl which is unsubstituted or substituted in the aryl moiety by 1 to 3 substituents selected from the group consisting of halogen, (C₁-C₃)alkyl and (C₁-C₃)alkoxy, which latter may in turn be substituted by 1 to 3 halogen atoms: and hetero-arylalkanoyl having one to three heteroatoms selected from O, S and N in the heteroaryl moiety and 2 to 10 carbon atoms in the alkanoyl moiety and which is unsubstituted or substituted in the heteroaryl moiety by 1 to 3 substituents selected from the group consisting of halogen, cyano, trifluoromethanesulphonyloxy, (C₁-C₃)alkyl, and (C₁-C₃)alkoxy, which latter may in turn be substituted by 1 to 3 halogen atoms, and the physiologically acceptable salts thereof.

In another embodiment, chromeno[4,3,2-de]isoquinoline compounds can be used as the D₁, dopamine receptor agonist administered in combination therapy with a D₂ dopamine receptor antagonist. Exemplary compounds that are used in the method and composition described herein include, but are not limited to compounds having Formula III: 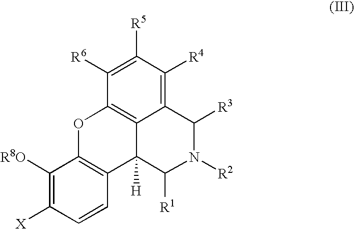
wherein R¹, R², and R³ are each independently selected from hydrogen, C₁-C₄ alkyl, and C₂-C₄ alkenyl, R⁸ is hydrogen, C₁-C₄ alkyl, acyl, or an optionally substituted phenoxy protecting group, X is hydrogen, halo including fluoro, chloro, bromo, and iodo, or a group of the formula —OR⁹ wherein R⁹ is hydrogen, C₁-C₄ alkyl, acyl, or an optionally substituted phenoxy protecting group, and R⁴, R⁵, and R⁶ are each independently selected from the group consisting of hydrogen, C₁-C₄ alkyl, phenyl, halo, and a group —OR wherein R is hydrogen, acyl, such as benzoyl, pivaloyl, and the like, or an optionally substituted phenyl protecting group, and when X is a group of the formula —OR⁹, the groups R⁵ and R⁹ can be taken together to form a group of the formula —CH₂— or —(CH₂)₂—. The compounds also comprise pharmaceutically acceptable salts thereof.

It is appreciated that compounds having Formula III are chiral. It is further appreciated that although a single enantiomer is depicted, each enantiomer alone and/or various mixtures of each enantiomer, including racemic mixtures, are contemplated, and may be included in the compounds, compositions, and methods described herein.

In this embodiment, “C₂-C₄ alkenyl” as used herein refers to branched or straight-chain alkenyl groups having two to four carbons, such as allyl, 2-butenyl, 3-butenyl, and vinyl.

In another embodiment, wherein compounds of Formula III are used in the method and composition described herein at least one of R₄, R₅, or R₆ is hydrogen. In another embodiment at least two of R₄, R₅, or R₆ are hydrogen.

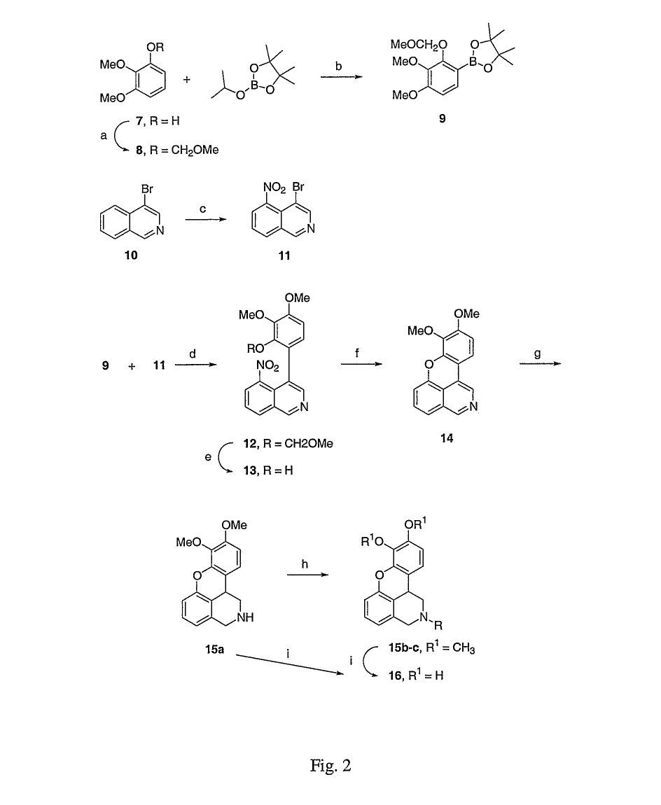

One compound of Formula III for use in the method and composition described herein is (O)-8,9-dihydroxy-1,2,3,11b-tetrahydrochromeno[4,3,2-de]isoquinoline hydrobromide (16a), denominated hereinafter as “dinoxyline.” Dinoxyline is synthesized from 2,3-dimethoxyphenol (7) and 4-bromoisoquinole (10), as depicted in FIG. 2. The phenolic group is protected as the methoxymethyl (“MOM”) derivative 8 followed by treatment with butyllithium, then with the substituted borolane illustrated, to afford the borolane derivative 9.

As shown in FIG. 2, this borolane derivative is then employed in a Pd-catalyzed Suzuki type cross coupling reaction with 5-nitro-4-bromoisoquinoline (11), prepared from bromoisoquinoline 10. The resulting coupling product 12 is then treated with toluenesulfonic acid in methanol to remove the MOM protecting group of the phenol. Treatment of this nitrophenol 13 with potassium carbonate in DMF at 80° C. leads to ring closure with loss of the nitro group, affording the basic tetracyclic chromenoisoquinoline nucleus 14. Catalytic hydrogenation effects reduction of the nitrogen-containing ring to yield 15a. Use of boron tribromide to cleave the methyl ether linkages gives the parent compound 16a.

It is apparent that by appropriate substitution on the isoquinoline ring a wide variety of substituted compounds can be obtained. Substitution onto the nitrogen atom in either 14 or 15a, followed by reduction will readily afford a series of compounds substituted with lower alkyl groups on the nitrogen atom. Likewise, the use of alkyl substituents on the 1, 3, 6, 7, or 8 positions of the nitroisoquinoline 11 leads to a variety of ring-substituted compounds. In addition, the 3-position of 14 can also be directly substituted with a variety of alkyl groups. Similarly, replacement of the 4-methoxy group of 9, in FIG. 2, with fluoro, chloro, or alkyl groups leads to the subject compounds with variations at X₉. When groups are present on the nucleus that are not stable to the catalytic hydrogenation conditions used to convert 14 to 15a, reduction can be accomplished using sodium cyanoborohydride at slightly acidic pH. Further, formation of the N-alkyl quaternary salts of derivatives of 14 gives compounds that are also easily reduced with sodium borohydride, leading to derivatives of 15a.

FIG. 2 also illustrates the synthesis of N-substituted chromenoisoquinolines 15 and 16. Compound 15a is N-alkylated under standard conditions to provide substituted derivatives. Alkylating agents, such as R-L, where R is methyl, ethyl, propyl, allyl, and the like, and L is a suitable leaving group such as halogen, methylsulfate, or a sulfonic acid derivative, are used to provide the corresponding N-alkyl derivatives. The aromatic methyl ethers of compounds 15 are then removed under standard conditions, such as upon treatment with BBr₃ and the like. It appreciated that N-alkylation may be followed by other chemical transformations to provide the substituted derivatives described herein. For example, alkylation with an allyl halide followed by hydrogenation of the allyl double bond provides the corresponding N-propyl derivative.

In another embodiment of compounds of formula III, where X is —OR⁹, R⁵ and R⁹ are different. In one aspect, one of R⁸ and R⁹ is hydrogen or acetyl and the other of R⁸ and R⁹ is selected from the group consisting of (C₃-C₂₀)alkanoyl, halo-(C₃-C₂₀)alkanoyl, (C₃-C₂₀)alkenoyl, (C₄-C₇)cycloalkanoyl, (C₃-C₆)-cycloalkyl(C₂-C₁₆)alkanoyl, aroyl which is unsubstituted or substituted by 1 to 3 substituents selected from the group consisting of halogen, cyano, trifluoromethanesulphonyloxy, (C₁-C₃)alkyl and (C₁-C₃)alkoxy, which latter may in turn be substituted by 1 to 3 halogen atoms, aryl(C₂-C₁₆)alkanoyl which is unsubstituted or substituted in the aryl moiety by 1 to 3 substituents selected from the group consisting of halogen, (C₁-C₃)alkyl and (C₁-C₃)alkoxy, which latter may in turn be substituted by 1 to 3 halogen atoms: and hetero-arylalkanoyl having one to three heteroatoms selected from O, S and N in the heteroaryl moiety and 2 to 10 carbon atoms in the alkanoyl moiety and which is unsubstituted or substituted in the heteroaryl moiety by 1 to 3 substituents selected from the group consisting of halogen, cyano, trifluoromethanesulphonyloxy, (C₁-C₃)alkyl, and (C₁-C₃)alkoxy, which latter may in turn be substituted by 1 to 3 halogen atoms, and the physiologically acceptable salts thereof.

In another embodiment, tetrahydronaphtho[1,2,3-de]isoquinoline compounds are used as the D₁ dopamine receptor agonist for co-administration with a D₂ dopamine receptor antagonist. Exemplary compounds for use in the method and composition described herein include, but are not limited to compounds having Formula IV: 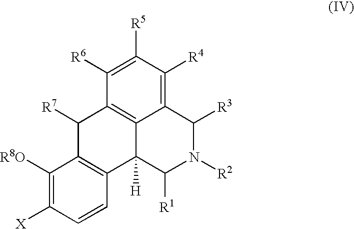
and pharmaceutically acceptable salts thereof, wherein R¹, R², and R³ are each independently selected from the group consisting of hydrogen, C₁-C₄ alkyl, and C₂-C₄ alkenyl; R⁴, R⁵, and R⁶ are each independently selected from the group consisting of hydrogen, C₁-C₄ alkyl, phenyl, halogen, and a group having the formula —OR, where R is hydrogen, acyl, such as benzoyl, pivaloyl, and the like, or an optionally substituted phenyl protecting group; R⁷ is selected from the group consisting of hydrogen, hydroxy, C₁-C₄ alkyl, C₂-C₄ alkenyl, C₁-C₄ alkoxy, and C₁-C₄ alkylthio; R⁸ is hydrogen, C₁-C₄ alkyl, acyl, or an optionally substituted phenyl protecting group; and X is hydrogen, fluoro, chloro, bromo, or iodo.

It is appreciated that compounds having Formula IV are chiral. It is further appreciated that although a single enantiomer is depicted, each enantiomer alone and/or various mixtures of each enantiomer, including racemic mixtures, are contemplated, and may be included in the compounds, compositions, and methods described herein.

In another embodiment of Formula IV, X is a group having the formula —OR⁹, where R⁹ is hydrogen, C₁-C₄ alkyl, acyl, or an optionally substituted phenyl protecting group; or the groups R⁸ and R⁹ are taken together to form a divalent group having the formula —CH₂— or —(CH₂)₂—.

In accordance with the method and composition described herein, the term “pharmaceutically acceptable salts” as used herein refers to those salts formed using organic or inorganic acids that are suitable for use in contact with the tissues of humans and lower animals without undue toxicity, irritation, allergic response, and the like. Acids suitable for forming pharmaceutically acceptable salts of biologically active compounds having amine functionality are well known in the art. The salts can be prepared according to conventional methods in situ during the final isolation and purification of the present compounds, or separately by reacting the isolated compounds in free base form with a suitable salt forming acid.

In accordance with the method and composition described herein, the term “phenoxy protecting group” as used herein refers to substituents on the phenolic oxygen which prevent undesired reactions and degradations during synthesis and which can be removed later without effect on other functional groups on the molecule. Such protecting groups and the methods for their application and removal are well known in the art. They include ethers, such as methyl, isopropyl, t-butyl, cyclopropylmethyl, cyclohexyl, allyl ethers and the like; alkoxyalkyl ethers such as methoxymethyl or methoxyethoxymethyl ethers and the like; alkylthioalkyl ethers such a methylthiomethyl ethers; tetrahydropyranyl ethers; arylalkyl ethers such as benzyl, o-nitrobenzyl, p-methoxybenzyl, 9-anthrylmethyl, 4-picolyl ethers and the like; trialkylsilyl ethers such as trimethylsilyl, triethylsilyl, t-butyldimethylsilyl, t-butyldiphenylsilyl ethers and the like; alkyl and aryl esters such as acetates, propionates, n-butyrates, isobutyrates, trimethylacetates, benzoates and the like; carbonates such as methyl, ethyl, 2,2,2-trichloroethyl, 2-trimethylsilylethyl, vinyl, benzyl and the like; and carbamates such as methyl, isobutyl, phenyl, benzyl, dimethyl and the like.

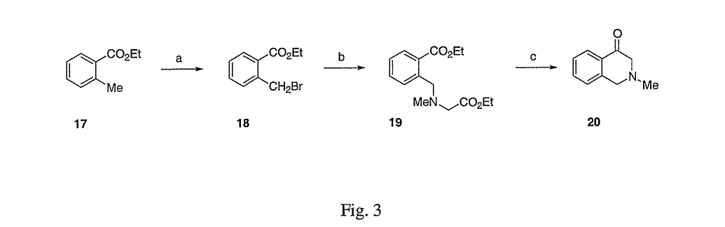

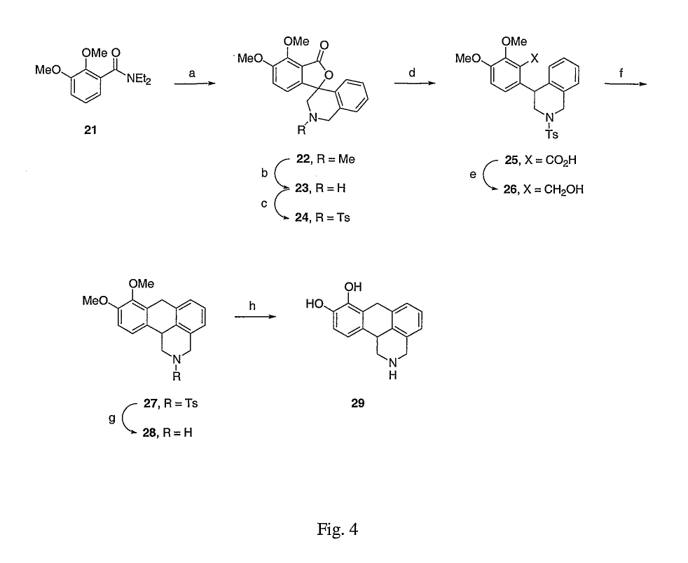

One compound for use in accordance with the method and composition described herein as a D₁ dopamine receptor agonist for co-administration with a D₂ dopamine receptor antagonist is (±)-8,9-dihydroxy-2,3,7,11b-tetrahydro-1H-naphtho-[1,2,3-de]-isoquinoline (29) denominated hereinafter as “dinapsoline.” Dinapsoline is synthesized from 2-methyl-2,3-dihydro-4(1H)-isoquinolone (20) according to the procedure depicted generally in FIGS. 3 and 4. Side chain bromination of ethyl 2-toluate (17) with NBS in the presence of benzoyl peroxide produced compound 18. Alkylation of sarcosine ethyl ester with compound 18 afforded compound 19, which after Dieckmann condensation and subsequent decarboxylation on acidic hydrolysis yielded compound 20.

As shown in FIG. 4, ortho-directed lithiation of 2,3-dimethoxy-N,N′-diethylbenzamide (21) with sec-butyllithium/TMEDA in ether at −78° C. and condensation of the lithiated species with compound 20 followed by treatment with p-toluene sulfonic acid at reflux gave spirolactone 22 in modest yield. N-Demethylation of 22 with 1-chloroethylchloroformate followed by methanolysis of the intermediate afforded compound 23, that on treatment with p-toluenesulfonyl chloride and triethylamine provided compound 24.

Early attempts to synthesize compound 24 directly by condensation of 2-p-toluenesulfonyl-2,3-dihydro-4(1H)-isoquinolone with lithiated compound 21 in THF or ether, followed by lactonization with acid provided only trace amounts (<5%) of compound 24. Enolization of 2-p-toluenesulfonyl-2,3-dihydro-4(1H)-isoquinolone under the basic reaction conditions is one possible explanation for the poor yield.

Hydrogenolysis of compound 24 in glacial acetic acid in the presence of 10% palladium on carbon gave compound 25 that on reduction with diborane afforded intermediate compound 26. Cyclization of compound 26 with concentrated sulfuric acid at low temperature provided compound 22. N-Detosylation of compound 22 with Na/Hg in methanol buffered with disodium hydrogen phosphate gave compound 28. Finally, compound 28 was treated with boron tribromide to effect methyl ether cleavage yielding dinapsoline (29) as its hydrobromide salt.

Alternatively, dinapsoline and compounds related to dinapsoline may also be synthesized according to the procedure described by Sattelkau, Qandil, and Nichols, “An efficient synthesis of the potent dopamine D₁, agonst dinapsoline by construction and selective reduction of 2′-azadimethoxybenzanthrone,” Syizthesis 2:262-66 (2001), the entirety of the description of which is incorporated herein by reference.

In another embodiment of compounds of formula IV, where X is —OR⁹, R⁸ and R⁹ are different. In one aspect, one of R⁸ and R⁹ is hydrogen or acetyl and the other of R⁸ and R⁹ is selected from the group consisting of (C₃-C₂₀)alkanoyl, halo-(C₃-C₂₀)alkanoyl, (C₃-C₂₀)alkenoyl, (C₄-C₇)cycloalkanoyl, (C₃-C₆)-cycloalkyl(C₂-C₁₆)alkanoyl, aroyl which is unsubstituted or substituted by 1 to 3 substituents selected from the group consisting of halogen, cyano, trifluoromethanesulphonyloxy, (C₁-C₃)alkyl and (C₁-C₃)alkoxy, which latter may in turn be substituted by 1 to 3 halogen atoms, aryl(C₂-C₁₆)alkanoyl which is unsubstituted or substituted in the aryl moiety by 1 to 3 substituents selected from the group consisting of halogen, (C₁-C₃)alkyl and (C₁-C₃)alkoxy, which latter may in turn be substituted by 1 to 3 halogen atoms: and hetero-arylalkanoyl having one to three heteroatoms selected from O, S and N in the heteroaryl moiety and 2 to 10 carbon atoms in the alkanoyl moiety and which is unsubstituted or substituted in the heteroaryl moiety by 1 to 3 substituents selected from the group consisting of halogen, cyano, trifluoromethanesulphonyloxy, (C₁-C₃)alkyl, and (C₁-C₃)alkoxy, which latter may in turn be substituted by 1 to 3 halogen atoms, and the physiologically acceptable salts thereof.

In another embodiment of compounds of formula IV, an optically active preparation is described.

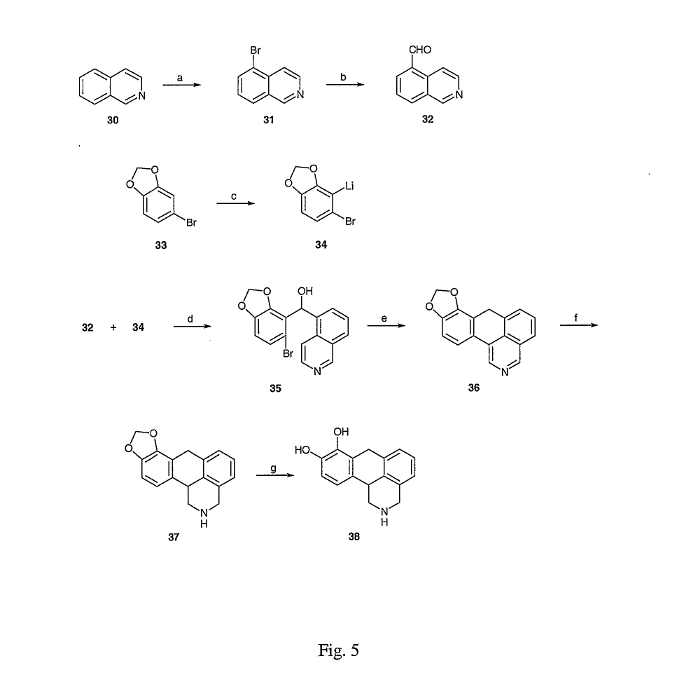

As illustrated in FIG. 5, compounds 35 may be prepared from optionally substituted isoquinolines 30, which generally undergo electrophilic substitution preferentially at the 5-position to give 5-bromo-isoquinolines 31. The bromination reaction is illustratively performed neat in the presence of a Lewis Acid catalyst, such as anhydrous aluminum chloride, or alternatively in an inert organic solvent, such as methylene chloride. 5-bromo-isoquinolines 31 can be trans-metallated to the corresponding 5-lithio-isoquinolines using n-butyl lithium in a suitable inert organic solvent such as THF, illustratively at a temperature less than about −50, or about −80° C., followed by alkylation, or optionally acylation, to form the corresponding 5-substituted isoquinolines. Acylation with DMF gives, followed by warming to room temperature and neutralization with an equivalent amount of mineral acid, gives 5-formyl-isoquinolines 32. Aldehyde 32 is reacted with 4-bromo-3-lithio-1,2-(methylenedioxy)benzene 34, prepared by conventional ortho-lithiation methods from the corresponding substituted benzene 33, to give 35.

Cyclization of 35 to the corresponding compounds 36 can be initiated by free radical initiated carbon-carbon bond formation, or by a variety of conventional reaction conditions. The carbon-carbon bond reaction is illustratively carried out with a hydrogen radical source such as trialkyltin hydride, triaryltin hydride, trialkylsilane, triarylsilane, and the like, and a radical initiator, such as 2,2′-azobisisobutylronitrile, sunlight, UV light, controlled potential cathodic (Pt), and the like in the presence of a proton source such as a mineral acid, such as sulfuric acid, hydrochloric acid, and the like, or an organic acid, such as acetic acid, trifluoroacetic acid, p-toluenesulfonic acid, and the like. Illustratively, 36 is prepared by treatment with tributyltin hydride and, 2,2′-azobisisobutylronitrile in the presence of acetic acid.

Compounds 36 are selectively reduced at the nitrogen bearing heterocyclic ring to give the corresponding tetrahydroisoquinolines 37. The selective ring reduction may be carried out by a number of different reduction methods such as sodium cyanoborohydride in an acidic medium in THF, hydride reducing agents such as L-SELECTRIDE or SUPERHYDRIDE, catalytic hydrogenation under elevated pressure, and the like. Conversion of the protected compounds 37 to diols 38 may be accomplished using boron tribromide in methylene chloride at low temperatures, such as less than about −60, or less than about −80° C. Compounds 38 may be isolated as the hydrobromide salt. The corresponding hydrochloride salt may also be prepared by using boron trichloride.

The substantially pure (+)-isomer and (−)-isomer of compounds 38 are prepared by chiral separation of the hydroxy-protected compounds 37, by forming a chiral salt, such as the (+)-dibenzoyl-D-tartaric acid salt of compounds 37, followed by removal of the protecting group as described herein.

In another embodiment, heterocyclic-fused phenanthridine compounds, such as thieno[1,2-a]phenanthridines, and the like, are used as the D₁ dopamine receptor agonist for administration in combination therapy with a D₂ dopamine receptor antagonist to patients with neurological disorders. Exemplary compounds for use in the methods and compositions described herein include, but are not limited to, compounds having Formula V: 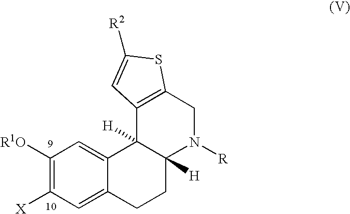
and pharmaceutically acceptable salts thereof; R is hydrogen or C₁-C₄ alkyl; R¹ is hydrogen, acyl, such as C₁-C₄ alkanoyl, benzoyl, pivaloyl, and the like, or a phenoxy protecting group; X is hydrogen, fluoro, chloro, bromo, iodo, or a group of the formula —OR³ wherein R³ is hydrogen, alkyl, acyl, or a phenoxy protecting group, provided that when X is a group of the formula-OR³, the groups R¹ and R³ can be taken together to form a —CH₂— group or a —(CH₂)₂— group, thus representing a methylenedioxy or ethylenedioxy functional group bridging the C-9 and C-10 positions; and R² is selected from the group consisting of hydrogen, C₁-C₄ alkyl, phenyl, fluoro, chloro, bromo, iodo, or a group —OR⁴ wherein R⁴ is hydrogen, alkyl, acyl, or a phenoxy protecting group.

It is appreciated that compounds having Formula V are chiral. It is further appreciated that although a single enantiomer is depicted, each enantiomer alone and/or various mixtures of each enantiomer, including racemic mixtures, are contemplated, and may be included in the compounds, compositions, and methods described herein.

Exemplary compounds of Formula V include, but are not limited to, ABT 431 (X═CH₃CO₂, R¹═CH₃CO, R²═CH₃(CH₂)₂, R═H) and A 86929 (X═OH, R¹═H, R²═CH₃(CH₂)₂, R═H).

In another embodiment, phenyltetrahydrobenzazepine compounds can be used as the D₁, dopamine receptor agonist for co-administration with a D₂ dopamine receptor antagonist. Exemplary compounds for use in the method and composition described herein include, but are not limited to compounds having Formula VI:

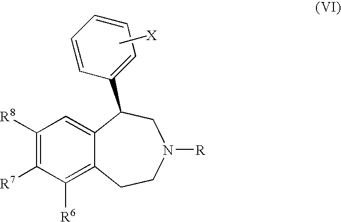

wherein R is hydrogen, alkyl, alkenyl, optionally substituted benzyl, or optionally substituted benzoyl; R⁶, R⁷, and R⁸ are each independently selected from hydrogen, halogen, hydroxy, alkyl, alkoxy, and acyloxy; and X is hydrogen, halogen, hydroxy, alkyl, alkoxy, or acyloxy. Illustrative compounds having the Formula VI include SKF 38393 (R⁶═H, R⁷═R⁸═OH, R═H, X═H), SKF 82958 (R⁶═Cl, R⁷═R⁸═OH, R═CH₂CH═CH₂, X═H), SKF 81297 (R⁶═Cl, R⁷═R⁹═OH, R═H, X═H, and described in Eur. J. Pharmacol. 188:335 (1990)), and SCH 23390 (R⁶═H, R⁷═Cl, R⁸═OH, R═CH₃, X═H).

It is appreciated that compounds having Formula VI are chiral. It is further appreciated that although a single enantiomer is depicted, each enantiomer alone and/or various mixtures of each enantiomer, including racemic mixtures, are contemplated, and may be included in the compounds, compositions, and methods described herein.

It is to be understood that other D₁ receptor agonists may be included in the compounds, compositions, and methods described herein, including but not limited to A68930 ((1R,3S)-1-aminomethyl-5,6-dihydroxy-3-phenylisochroman hydrochloride), A77636 ((1R,3S)-3-(1′-adamantyl)-1-aminomethyl-3,4-dihydro-5,6-dihydroxy-1H-2-benzopyran), and the like. A77636 may be prepared according to DeNinno et al., Eur. J. Pharmacol. 199:209-19 (1991) and/or DeNinno et al., J. Med. Chem. 34:2561-69 (1991), the disclosures of which are incorporated herein by reference.

In another embodiment, the dopamine D₁ receptor agonist is selected based on a predetermined half-life. Illustratively, dihydrexidine has a short-half life of about 30 min when given intravenously, and a functional half-life of about 3 hr when given subcutaneously. In contrast, dinapsoline has a 3 hr serum half-life with about 7-10 hr of functional activity.

The D₂ dopamine receptor antagonists that may be used in accordance with the methods and compositions described herein include typical or atypical families of antipsychotic agents. In one aspect, the typical antipsychotic agents include phenothiazines and non-phenothiazines such as loxapine, molindone, and the like. In another aspect, the atypical antipsychotic agents include the clozapine-like agents, and others, including aripiprazole, risperidone, amisulpiride, sertindole, and the like. Phenothiazines include, but are not limited to chlorpromazine, fluphenazine, mesoridazine, perphenazine, prochlorperazine, thioridazine, and trifluoperazine. Non-phenothiazines include, but are not limited to haloperidol, pimozide, and thiothixene. Clozapine-like agents include, but are not limited to the group consisting of olanzapine, clozapine, risperidone, sertindole, quetiapine, and ziprasidone. It appreciated that various combinations of the foregoing typical and atypical antipsychotic agents may be used in the methods and compositions described herein.

Any other antipsychotic agent, including any typical or atypical antipsychotic agent such as acetophenazine, acetophenazine maleate, triflupromazine, chlorprothixene, alentemol hydrobromide, alpertine, azaperone, batelapine maleate, benperidol, benzindopyrine hydrochloride, brofoxine, bromperidol, bromperidol decanoate, butaclamol hydrochloride, butaperazine, butaperazine maleate, carphenazine maleate, carvotroline hydrochloride, chlorpromazine hydrochloride, cinperene, cintriamide, clomacran phosphate, clopenthixol, clopimozide, clopipazan mesylate, chloroperone hydrochloride, clothiapine, clothixamide maleate, cyclophenazine hydrochloride, droperidol, etazolate hydrochloride, fenimide, flucindole, flumezapine, fluphenazine decanoate, fluphenazine enanthate, fluphenazine hydrochloride, fluspiperone, fluspirilene, flutroline, gevotroline hydrochloride, halopemide, haloperidol decanoate, iloperidone, imidoline hydrochloride, lenperone, mazapertine succinate, mesoridazine besylate, metiapine, milenperone, milipertine, molindone hydrochloride, naranol hydrochloride, neflumozide hydrochloride, ocaperidone, oxiperomide, penfluridol, pentiapine maleate, pinoxepin hydrochloride, pipamperone, piperacetazine, pipotiazine palmitate, piquindone hydrochloride, prochlorperazine edisylate, prochlorperazine maleate, promazine hydrochloride, remoxipride, remoxipride hydrochloride, rimcazole hydrochloride, seperidol hydrochloride, setoperone, spiperone, thioridazine hydrochloride, thiothixene hydrochloride, thioperidone hydrochloride, tiospirone hydrochloride, trifluoperazine hydrochloride, trifluperidol, triflupromazine hydrochloride, and ziprasidone hydrochloride, and the like, can also be used.

Olanzapine, 2-methyl-4-(4-methyl-1-piperazinyl)-10H-thieno[2,3-b][1,5]benzodiazepine, is a known compound and is described in U.S. Pat. No. 5,229,382, incorporated herein by reference. Clozapine, 8-chloro-11-(4-methyl-1-piperazinyl)-5H-dibenzo[b,e][1,4]diazepine, is described in U.S. Pat. No. 3,539,573 that is incorporated herein by reference. Risperidone, 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one is described in U.S. Pat. No. 4,804,663, that is incorporated by reference herein. Sertindole, 1-[2-[4-[5-chloro-1-(4-fluorophenyl)-1H-indol-3-yl]-1-piperidinyl]ethyl]imidazolidin-2-one, is described in U.S. Pats. Nos. 4,710,500, 5,112,838, and 5,238,945, incorporated by reference herein. Quetiapine, 5-[2-(4-dibenzo[b,f][1,4]thiazepin-11-yl-1-piperazinyl)ethoxy]ethanol, is described in U.S. Pat. No. 4,879,288 that is incorporated by reference herein. Ziprasidone, 5-[2-[4-(1,2-benzoisothiazol-3-yl)-1-piperazinyl]ethyl]-6-chloro-1,3-dihydro-2H-indol-2-one, is typically administered as the hydrochloride monohydrate. The compound is described in U.S. Pat. Nos. 4,831,031 and 5,312,925, incorporated by reference herein.

In another illustrative embodiment, pharmaceutical compositions are described herein. The pharmaceutical compositions include one or more dopamine D₁ receptor agonists, one or more dopamine D₂ receptor antagonists, and one or more pharmaceutically acceptable carriers, diluents, and/or excipients therefor. In one aspect, the amount of the dopamine D₁ receptor agonists and the amount of the dopamine D₂ receptor antagonists are each effective for treating a patient at risk of developing or having a neurological, psychotic, and/or psychiatric disorder.

As used herein, the term “effective amounts” refers to amounts of the compounds which prevent, reduce, or stabilize one or more of the clinical symptoms of disease in a patient at risk of developing or suffering from the neurological, psychotic, and/or psychiatric disorder. It is appreciated that the effective amount may improve the condition of a patient permanently or temporarily.

It is appreciated that the dopamine D₁ receptor agonists, for co-administration with the dopamine D₂ receptor antagonists, may vary in their selectivity for dopamine D₁ and D₂ receptors and receptor subtypes. In some embodiments, these dopamine receptor agonists exhibit activity at both the D₁ and D₂ dopamine receptor, with possible variation at the receptor subtypes. In one embodiment, this activity at the D₁ and D₂ dopamine receptor subtypes can be about equal. In another embodiment, this activity at the D₁ and D₂ dopamine receptor subtypes can be characterized by being selective for these two dopamine receptor subtypes as compared to other dopamine receptor subtypes. In this latter embodiment, the activity exhibited by the dopamine receptor agonists at the D₁ and D₂ dopamine receptor subtypes may be about equal or not. Among exemplary compounds, dihydrexidine is 10-fold D₁:D₂ selective and dinapsoline is 5-fold D₁:D₂ selective while dinoxyline has equally high affinity for both receptor subtypes. It is appreciated that substituted analogs of these compounds, as described herein by formulae I-IV, may each have a different selectivity for the D₁ and D₂ dopamine receptors and/or the various D₁ and D₂ dopamine receptor subtypes.

Typical dosages of the D₁ receptor agonist include dosage ranges from about 0.1 to about 100 mg/kg. It is appreciated that depending upon the route of administration, different ranges may be used. Illustratively, parenteral administration includes dosage ranges from about 0.1 to about 10, or from about 0.3 to about 3 mg/kg, and oral administration includes dosage ranges from about 0.1 to about 100, or form about 0.3 to about 30 mg/kg. Illustrative dosage for dihydrexidine and other hexahydrobenzo[a]phenanthridine compounds include 2 mg/15 min per day or 0.5 mg/kg dose (35 mg/15 min or 0.031 mg/kg/min per day by intravenous infusion. Other illustrative dosage for dihydrexidine and other hexahydrobenzo[a]phenanthridine compounds include 5-20 mg/15 min per day by subcutaneous infusion.

It is also appreciated that the dopamine D₂ receptor antagonists, for co-administration with the dopamine D₁ receptor agonists, may vary in their selectivity for dopamine D₁ and D₂ receptors and receptor subtypes. In some embodiments, these dopamine receptor antagonists exhibit activity at both the D₁ and D₂ dopamine receptor, with possible variation at the receptor subtypes. In one embodiment, this activity at the D₁ and D₂ dopamine receptor subtypes can be about equal. In another embodiment, this activity at the D₁ and D₂ dopamine receptor subtypes can be characterized by being selective for these two dopamine receptor subtypes as compared to other dopamine receptor subtypes. In this latter embodiment, the activity exhibited by the dopamine receptor antagonists at the D₁ and D₂ dopamine receptor subtypes may be about equal or not. In one aspect, the dopamine D₂ receptor antagonist does not exhibit significant binding at the dopamine D₁ receptor. In another aspect, the dopamine D₂ receptor antagonist does not exhibit significant functional activity at the dopamine D₁ receptor. In another aspect, the dopamine D₂ receptor antagonist does not exhibit significant agonist activity at the dopamine D₁ receptor. In another aspect, the dopamine D₂ receptor antagonist does not exhibit significant antagonist activity at the dopamine D₁ receptor.

Typical dosages of the D₂ receptor antagonist, such as olanzapine, fall in the ranges from about 0.25 to about 50 mg/day, about 1 to about 30 mg/day, and about 1 to about 25 mg day. Typical dosages of the D₂ receptor antagonist, such as clozapine, fall in the ranges from about 12.5 to about 900 mg/day, and about 150 to about 450 mg/day. Typical dosages of the D₂ receptor antagonist, such as risperidone, fall in the ranges from about 0.25 to about 16 mg/day, and about 2 to about 8 mg/day. Typical dosages of the D₂ receptor antagonist, such as sertindole, fall in the range from about 0.0001 to about 1 mg/day. Typical dosages of the D₂ receptor antagonist, such as quetiapine, fall in the ranges from about 1 to about 40 mg/day, and about 150 to about 450 mg/day. Typical dosages of the D₂ receptor antagonist, such as ziprasidone, fall in the ranges from about 5 to about 500 mg/day, and about 50 to about 100 mg/day. It is appreciated that such daily dosage regimens can be given advantageously once per day, or in two or more divided doses.

The compounds for use in the method and composition described herein can be formulated in conventional drug dosage forms, and can be in the same or different compositions. In accordance with the composition and method described herein “co-administration” means administration in the same or different compositions or in the same or different dosage forms or by the same or different routes of administration in any manner which provides effective levels of the active ingredients in the body at the same time. Combinations of D₁ dopamine receptor agonists and D₂ dopamine receptor antagonists can also be used in the “co-administration” protocols described above.

Various dosage forms are contemplated herein, including slid dosage forms such as tablets, pills, capsules, caplets, sublingual tablets, lozenges, and the like, liquid dosage forms such as syrups, elixirs, oral suspensions, and the like, among others.

Conventional process are used to prepare such various dosage forms described herein. Illustratively, pharmaceutical compositions contain the D₁ receptor agonist or the D₂ receptor antagonist is amounts in the range from about 0.5% to about 50% by weight. It is to be understood that the selection of active ingredient percentage weight is related to the dosage form selected.

Illustratively, capsules are prepared by mixing the compound with a suitable diluent and filling the proper amount of the mixture in a capsules, such as a gelatin capsule. Typical diluents include inert powdered substances such as starch, from a variety of sources, powdered cellulose, including crystalline and microcrystalline cellulose, sugars, including fructose, mannitol, and sucrose, grain flours, and other similar edible or palatable powders.

Illustratively, tablets are prepared by direct compression, by wet granulation, by dry granulation, and like processes. Such formulations typically incorporate diluents, binders, lubricants, disintegrators, and the like along with the compounds described herein. Typical diluents include, but are not limited to, various types of starch, lactose, mannitol, kaolin, calcium phosphate or sulfate, inorganic salts, such as sodium chloride, powdered sugar, powdered cellulose derivatives, among others. Typical tablet binders are substances such as starch, gelatin, sugars, such as lactose, fructose, glucose, polyethylene glycols, ethylcellulose, waxes, and like binders. Natural and/or synthetic gums may also be included in the tablet dosage forms described herein, including acacia, alginates, methylcellulose, polyvinylpyrrolidine, and the like.

Other optional ingredients useful in preparing the formulations described herein include lubricants, such as talc, magnesium and calcium stearate, stearic acid and hydrogenated vegetable oils, tablet disintegrators, such as starches, clays, celluloses, algins gums, corn and potato starches, methylcellulose, agars, bentonites, wood celluloses, powdered natural sponges, cation-exchange resins, alginic acids, guar gums, citrus pulp, carboxymethylcellulose, and sodium lauryl sulfate, and enteric coatings for timed release of the compounds described herein after exiting the stomach, such as cellulose acetate phthalate, polyvinyl acetate phthalate, hydroxypropyl methylcellulose phthalate and hydroxypropyl methylcellulose acetate succinate.

Routes of administration include, but are not limited to, parenteral administration such as intravenous, intramuscular, subcutaneous injection, subcutaneous depot, intraperitoneal, and the like; transdermal administration such as transdermal patchs, and the like; pumps such as implanted and indwelling pumps, and the like; intranasal administration such as aerosols, pulmonary aerosols, and the like; oral administration such as oral liquids and suspensions, tablets, pills, capsules, and the like; buccal administration such as sublingual tablets and lozenges, and the like; and vaginal administration and suppositories.

In one embodiment, the drug dosage forms are formulated for oral ingestion by the use of such dosage forms as syrups, sprays, or other liquid dosage forms, a gel-seal, or a capsule or caplet. Syrups for either use may be flavored or unflavored and may be formulated using a buffered aqueous solution of the active ingredients as a base with added caloric or non-caloric sweeteners, flavor oils and pharmaceutically acceptable surfactant/dispersants. Other liquid dosage forms, including liquid solutions or sprays can be prepared in a similar manner and can be administered buccally, sublingually, or by oral ingestion.

In one embodiment, buccal and sublingual administration is used and comprises contacting the oral and pharyngeal mucosa of the patient with the D₁ agonist and the D₂ antagonist either in a pharmaceutically acceptable liquid dosage form, such as a syrup or a spray, or in a saliva-soluble dosage form which is held in the patient's mouth to form a saliva solution. Exemplary of saliva-soluble dosage forms are lozenges, tablets, and the like.

In one embodiment, lozenges can be prepared, for example, by art-recognized techniques for forming compressed tablets where the active ingredients are dispersed on a compressible solid carrier, optionally combined with any appropriate tableting aids such as a lubricant (e.g., magnesium-stearate) and are compressed into tablets. The solid carrier component for such tableting formulations can be a saliva-soluble solid, such as a cold water-soluble starch or a monosaccharide or disaccharide, so that the lozenge will readily dissolve in the mouth to release the active ingredients. The pH of the above-described formulations can range from about 4 to about 8.5. Lozenges can also be prepared utilizing other art-recognized solid unitary dosage formulation techniques.

In another embodiment, tablets are used. Tablets can be prepared in a manner similar to that described for preparation of lozenges or by other art-recognized techniques for forming compressed tablets such as chewable vitamins. Tablets can be prepared by direct compression, by wet granulation, or by dry granulation, and usually incorporate diluents, binders, lubricants and disintegrators as well as the active ingredients. Typical diluents include, for example, starches, lactose, mannitol, kaolin, calcium phosphate or sulfate, inorganic salts such as sodium chloride, powdered sugar, microcrystalline cellulose, carboxymethyl cellulose, and powdered cellulose derivatives.

Typical binders include starches, gelatin and sugars such as lactose, fructose, glucose and the like, natural and synthetic gums, including acacia, alginates, methylcellulose, polyvinylpyrrolidine and the like, polyethylene glycol, ethylcellulose, and waxes. Typical lubricants include talc, magnesium and calcium stearate, stearic acid, and hydrogenated vegetable oils. Typical tablet disintegrators include starches, clays, celluloses, algins and gums, corn and potato starches, methylcellulose, agar, bentonite, wood cellulose, powdered natural sponge, cation-exchange resins, alginic acid, guar gum, citrus pulp, carboxymethylcellulose, and sodium lauryl sulfate. Tablets can be coated with sugar as a flavor and sealant, or tablets can be formulated as chewable tablets, by using substances such as mannitol in the formulation, according to formulation methods known in the art, or as instantly dissolving tablet-like formulations according to known methods.

Solid dosage forms for oral ingestion administration also include such dosage forms as caplets, capsules, and gel-seals. Such solid dosage forms can be prepared using standard tableting protocols and excipients to provide capsules, caplets, or gel-seals containing the active ingredients. The usual diluents for capsules and caplets include inert powdered substances such as starch of many different kinds, powdered cellulose, especially crystalline and microcrystalline cellulose, sugars such as fructose, mannitol and sucrose, grain flours and similar edible powders. Any of the solid dosage forms for use in accordance with the invention, including lozenges and tablets, may be in a form adapted for sustained release of the active ingredients.

In another embodiment, parenteral administration is used. Parenteral administration can be accomplished by injection of a liquid dosage form, such as by injection of a solution of the D₁, agonist and the D₂ antagonist dissolved in a pharmaceutically acceptable buffer. Such parenteral administration can be intradermal, subcutaneous, intramuscular, intraperitoneal, or intravenous. Transdermal patches known in the art can also be used.

In accordance with one embodiment, a pharmaceutical composition is provided comprising effective amounts of the active ingredients, and a pharmaceutically acceptable carrier therefor. A “pharmaceutically acceptable carrier” for use in accordance with the method and composition described herein is compatible with other reagents in the pharmaceutical composition and is not deleterious to the patient. The pharmaceutically acceptable carrier formulations for pharmaceutical compositions adapted for oral ingestion or buccal/sublingual administration including lozenges, tablets, capsules, caplets, gel-seals, and liquid dosage forms, including syrups, sprays, and other liquid dosage forms, have been described above.

The active ingredients can also be adapted for parenteral administration in accordance with this invention using a pharmaceutically acceptable carrier adapted for use in a liquid dose form. Thus, the active ingredients can be administered dissolved in a buffered aqueous solution typically containing a stabilizing amount (1-5% by weight) of albumin or blood serum. Such a liquid solution can be in the form of a clarified solution or a suspension. Exemplary of a buffered solution administered parenterally in accordance with this invention is phosphate buffered saline prepared as follows:

A concentrated (20×) solution of phosphate buffered saline (PBS) is prepared by dissolving the following reagents in sufficient water to make 1,000 mL of solution: sodium chloride, 160 grams; potassium chloride, 4.0 grams; sodium hydrogen phosphate, 23 grams; potassium dihydrogen phosphate, 4.0 grams; and optionally phenol red powder, 0.4 grams. The solution is sterilized by autoclaving at 15 pounds of pressure for 15 minutes and is then diluted with additional water to a single strength concentration prior to use.

In another embodiment, aerosol administration of the active ingredients can be used. Aerosol and dry powder formulations for delivery to the lungs and devices for delivering such formulations to the endobronchial space of the airways of a patient are described in U.S. Pat. No. 6,387,886, incorporated herein by reference, and in Zeng et al., Int'l J. Pharm., vol. 191: 131-140 and Odumu et al., Pharm. Res., vol. 19: 1009-1012, although any other art-recognized formulations or delivery devices can be used. The D₁ dopamine receptor agonist and the D₂ dopamine receptor antagonist can be in the form of an aerosol or a dry powder diluted in, for example, water or saline, the diluted solution having a pH of, for example, between about 5.5 and about 7.0.

In one embodiment the solution can be delivered using a nebulized aerosol formulation, nebulized by a jet, ultrasonic or electronic nebulizer, capable of producing an aerosol with a particle size of between about 1 and about 5 microns, for example. In another embodiment the formulation can be administered in dry powder form where the active ingredient comprises part or all of the mass of the powder delivered. In this embodiment, the formulation can be delivered using a dry powder or metered dose inhaler, or the like. The powder can have average diameters ranging from about 1 to about 5 microns formed by media milling, jet milling, spray drying, or particle precipitation techniques.

The doses of the D₁ agonist and the D₂ antagonist for use in the method and composition depend on many factors, including the indication being treated and the overall condition of the patient. For example, in one embodiment effective amounts of the present compounds range from about 1.0 ng/kg to about 15 mg/kg of body weight. In another embodiment effective amounts range from about 50 ng/kg to about 10 mg/kg of body weight. In another embodiment effective amounts range from about 200 ng/kg to about 5 mg/kg of body weight. In another embodiment effective amounts range from about 300 ng/kg to about 3 mg/kg of body weight. In another embodiment effective amounts range from about 500 ng/kg to about 1 mg/kg of body weight. In another embodiment effective amounts range from about 1 μg/kg to about 0.5 mg/kg of body weight. In general, treatment regimens utilizing compounds in accordance with the present invention comprise administration of from about 10 ng to about 1 gram of the compounds for use in the method and composition described herein per day in multiple doses or in a single dose. Effective amounts of the compounds can be administered using any regimen such as twice daily, for at least one day to about twenty-one days.

The pharmaceutical compositions described herein may also include additional substances that may enhance the effectiveness of the methods described herein, including but not limited to acetylcholine esterase inhibitors, AAD, AAAD, or catechol-O-methyltransferase (COMT) inhibitors. It is appreciated that such inhibitors are used in combination with traditional levodopa therapy.

In another embodiment, the methods described herein are used to treat various stages of the diseases responsive to combination therapy using a D₁ receptor agonist and a D₂ receptor antagonist. In one embodiment, the compounds and compositions, and the methods for administering the compounds and compositions described herein, are used to treat all stages of diseases such as Parkinson's disease. In one illustrative variation, the compounds and compositions, and the methods for administering the compounds and compositions described herein, are used to treat advanced stages of diseases such as Parkinson's disease. It is appreciated that early stages of Parkinson's disease may also be treatable with carbidopa, levodopa, pramipexole, ropinirole, entacapone, pergolide, apomorphine, and combinations thereof. It is further appreciated that delaying introduction of levodopa therapy in conjunction with various treatment protocols may be advantageous.

### EXAMPLES

The following examples are illustrative of the compounds for use in the presently claimed methods and compositions and are not intended to limit the invention to the disclosed compounds. Other compounds that can be used in accordance with the claimed method include those compounds described in U.S. Pat. Nos. 5,047,536, 5,420,134, 5,959,110, 6,413,977, and 6,147,072. Each of these patents is incorporated herein by reference. Obvious variations and modifications of the exemplified compounds are also intended to be within the scope of the compounds, compositions, and methods described herein.

With reference to the experimental procedures described herein, unless otherwise indicated, the following procedures were used where applicable. Solvent removal was accomplished by rotary evaporation under reduced pressure. Melting points were determined with a Thomas-Hoover melting point apparatus and are uncorrected. ¹H NMR spectra chemical shifts are reported in values (ppm) relative to TMS. The IR spectra were recorded as KBr pellets or as a liquid film. Mass spectra were obtained using chemical ionization (CIMS). When anhydrous conditions were required, THF was distilled from benzophenone-sodium ketyl under N₂ immediately before use, and 1,2-Dichloroethane was distilled from phosphorous pentoxide before use.

### Example 1

### Dihydrexidine (6a)

2-(N-Benzyl-N-benzoyl)-6,7-dimethoxy-3,4-dihydro-2-napthylamine (2a). To a solution of 4.50 g (21.8 mmol) of 6,7-dimethoxy-β-tetralone (1) in 100 mL of toluene was added 2.46 g (23 mmol) of benzylamine. The reaction was heated at reflux overnight under N₂ with continuous water removal. The reaction was cooled, and the solvent was removed to yield N-benzyl enamine as a brown oil.

This residue was dissolved in 80 mL of CH₂Cl₂, and to this was added 2.43 g (24 mmol) of triethylamine, and the solution was cooled in an ice bath. Benzoyl chloride (3.37 g, 24 mmol) was then dissolved in 15 mL of CH₂Cl₂ and this solution was then added dropwise to the cold stirring N-benzyl enamine solution. After complete addition the reaction was allowed to warm to room temperature and was left to stir overnight. The mixture was then washed successively with 2×50 mL of 5% aqueous HCl, 2×50 mL of 1 N NaOH, saturated NaCl solution, and was then dried over MgSO₄. After filtration, the filtrate was concentrated. Crystallization from diethyl ether gave 5.6 g (64%) of enamide 2: mp 109-110° C.; IR (KBr) 1620 cm⁻¹; CIMS (isobutane, M+1) 400; ¹H-NMR (CDCl₃) δ 7.64 (m, 2, ArH), 7.33 (m, 8, ArH), 6.52 (s, 1, ArH), 6.38 (s, 1, ArH), 6.05 (s, 1, ArCH), 4.98 (s, 2, ArCH₂ N), 3.80 (s, 3, OCH₃), 3.78 (s, 3, OCH₃), 2.47 (t, 2, CH₂, J=8.1 Hz), 2.11 (t, 2, CH₂, J=8.1 Hz).

Trans-6-benzyl-10,11-dimethoxy-5,6,6a,7,8,12b-hexahydrobenzo[a]phenanthridine-5-one (3a). A solution of 3.14 g (7.85 mmol) of the 6,7-dimethoxyenamide 2, in 300 mL of THF, was introduced into an Ace Glass 250 mL photochemical reactor. This solution was stirred while irradiating for 5 hours with a 450 watt Hanovia medium pressure, quartz, mercury-vapor lamp seated in a water cooled, quartz immersion well. The solution was concentrated and crystallized from ether to provide 1.345 g (42.9%) of 3a: mp 183-186° C.; IR (KBr) 1655, 1640 cm⁻¹; CIMS (isobutane, M+1) 400; ¹H-NMR (CDCl₃) δ 8.19 (m, 1 ArH), 7.52 (m, 1, ArH), 7.46 (m, 2, ArH), 7.26 (m, 5, ArH), 6.92 (s, 1, ArH), 6.63 (s, 1, ArH), 5.35 (d, 1, ArCH₂N, J=16.0 Hz), 4.78 (d, 1, ArCH₂ N, J=16.0 Hz), 4.37 (d, 1, Ar₂CH, J=11.3 Hz), 3.89 (s, 3, OCH₃), 3.88 (s, 3, OCH₃), 3.80 (m, 1 CHN), 2.67 (m, 2, ArCH₂), 2.25 (m, 1, CH₂CN), 1.75 (m, 1, CH₂CN).

Trans-6-benzyl-10,11-dimethoxy-5,6,6a,7,8,12b-hexahydrobenzo[a]phenanthridine hydrochloride (4a). A solution of 1.20 g (3 mmol) of 3a, in 100 mL of dry THF was cooled in an ice-salt bath and 6.0 mL of 1 M BH₃ was added via syringe. The reaction was heated at reflux overnight. Water (10 mL) was added dropwise, and the reaction mixture was concentrated by distillation at atmospheric pressure. The residue was stirred with 50 mL of toluene, 1.0 mL of methane sulfonic acid was added, and the mixture was heated with stirring on the steam bath for one hour. The mixture was diluted with 40 mL of water and the aqueous layer was separated. The toluene was extracted several times with water, and the aqueous layers were combined. After basification of the aqueous phase with conc. ammonium hydroxide, the free base was extracted into 5×25 mL of CH₂Cl₂. This organic extract was washed with saturated NaCl solution, and dried over MgSO₄. After filtration, the organic solution was concentrated, the residue was taken up into ethanol, and carefully acidified with concentrated HCl. After drying several times by azeotropic distillation of ethanol, crystallization from ethanol afforded 0.97 g (76.5%) of the salt 4a: mp 235-237° C.; CIMS (NH₃, M+1) 386; ¹H-NMR (CDCl₃, free base) δ 7.37 (m, 9 ArH), 6.89 (s, 1, ArH), 6.74 (s, 1, ArH), 4.07 (d, 1, Ar₂CH, J=10.7 (Hz), 3.90 (s, 3, OCH₃), 3.82 (m, 2, ArCH₂N), 3.79 (s, 3, OCH₃), 3.52 (d, 1 ArCH₂N, J=15.3 Hz), 3.30 (d, 1, ArCH₂), J=13.1 Hz), 2.86 (m, 2, CHN, ArCH₂), 2.30 (m, 2, ArCH₂, CH₂CN), 1.95 (m, 1, CH₂CN).

Trans-10,11-dimethoxy-5,6,6a,7,8,12b-hexahydrobenzo[a]phenanthridine hydrochloride (5a). A solution of 0.201 g (0.48 mmol) of the 6-benzyl hydrochloride salt 4a in 50 mL of 95% ethanol containing 50 mg of 10% Pd—C catalyst was shaken at room temperature under 50 psig of H₂ for 8 hours. After removal of the catalyst by filtration, the solution was concentrated to dryness and the residue was recrystallized from acetonitrile to afford 0.119 g (75%) of 5a as a crystalline salt: mp 243-244° C.; CIMS (NH₃, M+1) 296; ¹H-NMR (CDCl₃, free base) δ 7.46 (d, 1, ArH, J=6.1 Hz), 7.24 (m, 3, ArH), 6.91 (s, 1, ArH), 6.74 (s, 1, ArH), 4.09 (s, 2, ArCH₂N), 3.88 (s, 3, OCH₃), 3.78 (m, 4, OCH₃, Ar₂CH), 2.87 (m, 3, CHN, ArCH₂), 2.17 (m, 1, CH₂CN), 1.61 (m, 2, NH, CH₂CN).

Trans-10,11-dihydroxy-5,6,6a,7,8,12b-hexahydrobenzo[a]phenanthridine hydrochloride (dihydrexidine, 6a). A suspension of 0.109 g (0.33 mmol) of the 10,11-dimethoxy salt 5a, in 1.5 mL of 48% IBr, was heated at reflux, under N₂, for 3 hours. The reaction mixture was concentrated to dryness under high vacuum. This material was dissolved in water and neutralized to the free base with NaHCO₃, while cooling the solution in an ice bath. The free base was extracted into chloroform, dried, filtered, and concentrated in vacuo. The residue was dissolved in ethanol and carefully neutralized with conc. HCl. After removal of the volatiles, the salt was crystallized as a solvate from methanol. This afforded 30 mg (25.2%) of 6, solvated with a stoichiometry of 1 molecule of amine salt and 1.8 molecules of CH₃OH, as pale yellow crystals: mp 195° C.; CIMS (isobutane, M+1) 268; ¹H-NMR (DMSO, HBr salt) δ 9.40 (bs, 1, ⁺NH₂), 9.22 (bs, 1, ⁺NH₂), 8.76 (bs, 2, OH), 7.38 (m, 4, ArH), 6.72 (s, 1, ArH), 6.63 (s, 1, ArH), 4.40 (s, 2, ArCH₂N⁺), 4.16 (d, 1, Ar₂CH, J=11.1 Hz), 3.00 (m, 1, CHN⁺), 2.75 (m, 2, ArCH₂), 2.17 (m, 1, CH₂CN⁺), 1.90 (m, 1, CH₂CN⁺).

### Example 2

### 2-Methyldihydrexidine (6b)

2-(N-benzyl-N-4-methylbenzoyl)-6,7-dimethoxy-3,4-dihydro-2-naphthylamine (2b). To a solution of 4.015 g (19.5 mmol) of 6,7-dimethoxy-p-tetralone in 100 mL of toluene was added 2.139 g (1.025 equiv.) of benzylamine. The reaction was heated at reflux overnight under N₂ with continuous water removal. The reaction was cooled and the solvent was removed to yield N-benzyl enamine as a brown oil.

The 4-methylbenzoyl chloride acylating agent was prepared by suspending 3.314 g (24.3 mmol) of 4-toluic acid in 200 mL benzene. To this solution was added 2.0 equivalents (4.25 mL) of oxalyl chloride, dropwise via a pressure-equalizing dropping funnel at O° C. Catalytic DMF (2-3 drops) was added to the reaction mixture and the ice bath was removed. The progress of the reaction was monitored using infrared spectroscopy. The solvent was removed and the residual oil was held under high vacuum overnight.

The resulting N-benzyl enamine residue was dissolved in 100 mL of CH₂Cl₂, and to this solution was added 2.02 g (19.96 mmol) of triethylamine at O° C. The 4-methylbenzoyl chloride (3.087 g, 19.96 mmol) was dissolved in 20 mL CH₂Cl₂ and this solution was added dropwise to the cold, stirring N-benzyl enamine solution. The reaction was allowed to warm to room temperature and was left to stir under N₂ overnight. The reaction mixture was washed successively with 2×30 mL of 5% aqueous HCl, 2×30 mL of saturated sodium bicarbonate solution, saturated NaCl solution, and was dried over MgSO₄. After filtration, the filtrate was concentrated. Crystallization from diethyl ether gave 5.575 g (69.3%) of the enamide 2b: mp 96-98° C.; CIMS (isobutane, M+1) 414; ¹H-NMR (CDCl₃) δ 7.59 (d, 2, ArH), 7.46 (m, 3, ArH), 7.35 (m, 3, ArH), 7.20 (d, 2, ArH), 6.60 (s, 1, ArH), 6.45 (s, 1, ArH), 6.18 (s, 1, ArCH), 5.01 (s, 2, ArCH₂N), 3.80 (S, 3, OCH₃), 3.78 (s, 3, OCH₃), 2.53 (t, 2, ArCH₂), 2.37 (s, 3, ArCH₃), 2.16 (t, 2, CH₂).

Trans-2-methyl-6-benzyl-10,11-dimethoxy-5,6,6a,7,8,12b-hexahydrobenzo[a]phenanthridine-5-one (3b). A solution of 4.80 g (11.62 mmol) of the 6,7-dimethoxyenamide 2b, in 500 mL of THF, was introduced to an Ace Glass 500 mL photochemical reactor. This solution was stirred while irradiating for 2 hours with a 450 watt Hanovia medium pressure, quartz, mercury-vapor lamp seated in a water cooled, quartz immersion well. The solution was concentrated and crystallized from diethyl ether to provide 2.433 (50.7%) of the 10,11-dimethoxy lactam 3b: mp 183-195° C.; CIMS (isobutane, M+1) 414; ¹H-NMR (CDCl₃) δ 8.13 (d, 1, ArH), 7.30 (s, 1, ArH), 7.23 (m, 6, ArH), 6.93 (s, 1, ArH), 6.63 (s, 1, ArH), 5.38 (d, 1, ArCH₂N), 5.30 (d, 1, ArCH₂N), 4.34 (d, 1, Ar₂CH, J=11.4 Hz), 3.89 (s, 3, OCH₃), 3.88 (s, 3, OCH₃), 3.76 (m, 1, CHN), 2.68 (m, 2, ArCH₂), 2.37 (s, 3, ArCH₃), 2.25 (m, 1, CH₂CN), 1.75 (m, 1, CH₂CN).

Trans-2-methyl-6-benzyl-10,11-dimethoxy-5,6,6a,7,8,12b-hexahydrobenzo[a]phenanthridine hydrochloride (4b). A solution of 1.349 g (3.27 mmol) of the lactam 3b, in 100 mL dry THF was cooled in an ice-salt bath and 4.0 equivalents (13.0 nL) of 1.0 molar BH₃ was added through a syringe. The reaction was heated at reflux under nitrogen overnight. Methanol (10 mL) was added dropwise to the reaction mixture and reflux was continued for 1 hour. The solvent was removed. The residue was chased two times with methanol and twice with ethanol. The residue was placed under high vacuum (0.05 mm Hg) overnight. The residue was dissolved in ethanol and was carefully acidified with concentrated HCl. The volatiles were removed and the product was crystallized from ethanol to afford 1.123 g (78.9%) of the hydrochloride salt 4b: mp 220-223° C.; CIMS (isobutane, M+1) 400;

¹H-NMR (CDCl₃, free base) δ 7.37 (d, 2, ArH), 7.33 (m, 2, ArH), 7.26 (m, 1, ArH), 7.22 (s, 1, ArH), 7.02 (d, 1, ArH), 6.98 (d, 1, ArH), 6.89 (s, 1, ArH), 6.72 (s, 1, ArH), 4.02 (d, 1, Ar₂CH, J=10.81 Hz), 3.88 (s, 3, OCH₃), 3.86 (d, 1, ArCH₂N), 3.82 (m, 1, ArCH₂N), 3.78 (s, 3, OCH₃), 3.50 (d, 1, ArCH₂N), 3.30 (d, 1, ArCH₂N), 2.87 (m, 1, ArCH₂), 2.82 (m, 1, CHN), 2.34 (m, 1, CH₂CN), 2.32 (s, 3, ArCH₃), 2.20 (m, 1, ArCH₂), 1.93 (m, 1, CH₂CN).

Trans-2-methyl-10,11-dimethoxy-5,6,6a,7,8,12b-hexahydrobenzo[a]phenanthridine hydrochloride (5b). A solution of 0.760 g (1.75 mmol) of the 6-benzyl derivative 4b in 100 mL of 95% ethanol containing 150 mg of 10% Pd/C catalyst was shaken at room temperature under 50 psig of H₂ for 8 hours. After removal of the catalyst by filtration through Celite, the solution was concentrated to dryness and the residue was recrystallized from acetonitrile to afford 0.520 g (86.2%) of 5b as a crystalline salt: mp 238-239° C.; CIMS (isobutane, M+1) 310; ¹H-NMR (DMSO, HCl salt) δ 10.04 (s, 1, NH), 7.29 (d, 1, ArH), 7.16 (m, 2, ArH), 6.88 (s, 1, ArH), 6.84 (s, 1, ArH), 4.31 (s, 2, ArCH₂N), 4.23 (d, 1, Ar₂CH, J=10.8 Hz), 3.76 (s, 3, OCH₃), 3.70 (s, 3, OCH₃), 2.91 (m, 2, ArCH₂), 2.80 (m, 1, CHN), 2.49 (s, 3, ArCH₃), 2.30 (m, 1, CH₂CN), 2.09 (m, 1, CH₂CN).

Trans-2-methyl-10,11-dihydroxy-5,6,6a,7,8,12b-hexahydrobenzo [a]phenanthridine hydrochloride (6b). The 10,11-dimethoxy hydrochloride salt 5b (0.394 g, 1.140 mmol) was converted to its free base. The free base was dissolved in 35 mL of CH₂Cl₂ and the solution was cooled to −78° C. A 1.0 molar solution of BBr₃ (4.0 eq., 4.56 mL) was added slowly through a syringe. The reaction was stirred under N₂ overnight with concomitant warming to room temperature. Methanol (7.0 mL) was added to the reaction mixture and the solvent was removed. The residue was placed under high vacuum (0.05 mm Hg) overnight. The residue was dissolved in water and was carefully neutralized to its free base initially with sodium bicarbonate and finally with ammonium hydroxide (1-2 drops). The free base was isolated by suction filtration and was washed with cold water. The filtrate was extracted several times with dichloromethane and the organic extracts were dried, filtered, and concentrated. The filter cake and the organic residue were combined, dissolved in ethanol, and carefully acidified with concentrated HCl. After removal of the volatiles, the HCl salt was crystallized as a solvate from methanol in a yield of 0.185 g (51%) of 6b: mp 190° C. (dec.); CIMS (isobutane, M+1) 282; ¹H-NMR (DMSO, HCl salt) δ 9.52 (s, 1, NM), 8.87 (d, 2, OH), 7.27 (d, 1, ArH), 7.20 (s, 1, ArH), 7.15 (d, 1, ArH), 6.72 (s, 1, ArH), 6.60 (s, 1, ArH), 4.32 (s, 2, ArCH₂N), 4.10 (d, 1, ArCH₂CH, J=11.26 Hz), 2.90 (m, 1, CHN), 2.70 (m, 2, ArCH₂), 2.32 (s, 3, ArCH₃), 2.13 (m, 1, CH₂CN), 1.88 (m, 1, CH₂CN).

### Example 3

### 3-Methyldihydrexidine (6c)

2-(N-benzyl-N-3-methylbenzoyl)-6,7-dimethoxy-3,4-dihydro-2-naphthylamine (2c). To a solution of 3.504 g (17.0 mmol) of 6,7-dimethoxy-β-tetralone in 100 mL of toluene was added 1.870 g (1.025 equivalents) of benzylamine. The reaction was heated at reflux overnight under N₂ with continuous water removal. The reaction was cooled and the solvent was removed to yield the N-benzyl enamine as a brown oil.

The 3-methylbenzoyl chloride acylating agent was prepared by suspending 3.016 g (22.0 mmol) of 3-toluic acid in 100 mL benzene. To this solution was added 2.0 equivalents (3.84 mL) of oxalyl chloride, dropwise with a pressure-equalizing dropping funnel at 0° C. Catalytic DMF (2-3 drops) was added to the reaction mixture and the ice bath was removed. The progress of the reaction was monitored using infrared spectroscopy. The solvent was removed and the residual oil was held under high vacuum overnight.

The resulting N-benzyl enamine residue was dissolved in 100 mL of CH₂Cl₂, and to this solution was added 1.763 g (17.42 mmol) of triethylamine at 0° C. The 3-methylbenzoyl chloride (2.759 g, 17.84 mmol) was dissolved in 20 mL CH₂Cl₂ and this solution was added dropwise to the cold, stirring N-benzyl enamine solution. The reaction was allowed to warm to room temperature and was left to stir under N₂ overnight. The reaction mixture was washed successively with 2×30 mL of 5% aqueous HCl, 2×30 mL of saturated sodium bicarbonate solution, saturated NaCl solution, and was dried over MgSO₄. After filtration, the filtrate was concentrated. Crystallization from diethyl ether gave 4.431 g (63.1%) of the enamide 2c: mp 96-97° C.; CIMS (isobutane, M+1) 414; ¹H-NMR (CDCl₃) δ 7.36 (s, 1, ArH), 7.26 (m, 3, ArH), 7.20 (m, 5, ArH), 6.50 (s, 1, ArH), 6.40 (s, 1, ArH), 6.05 (s, 1, ArCH), 4.95 (s, 2, ArCH₂N), 3.75 (s, 3, OCH₃), 3.74 (s, 3, OCH₃), 2.43 (t, 2, ArCH₂), 2.28 (s, 3, ArCH₃), 2.07 (t, 2, CH₂).

Trans-3-methyl-6-benzyl-10,11-dimethoxy-5,6,6a,7,8,12b-hexahydrobenzo[a]phenanthridine-5-one (3c). A solution of 1.922 g (4.65 mmol) of the 6,7-dimethoxyenamide 2c, in 500 mL of THF, was introduced to an Ace Glass 500 mL photochemical reactor. This solution was stirred while irradiating for 5 hours with a 450 watt Hanovia medium pressure, quartz, mercury-vapor lamp seated in a water-cooled, quartz immersion well. The solution was concentrated and crystallized from diethyl ether to provide 0.835 g (43.4%) of lactam 3c: mp 154-157° C.; CIMS (isobutane, M+1) 414; ¹H-NMR (CDCl₃) δ 7.94 (s, 1, ArH), 7.34 (d, 1, ArH), 7.17 (m, 6, ArH), 6.84 (s, 1, ArH), 6.54 (s, 1, ArH), 5.28 (d, 1, ArCH₂N), 4.66 (d, 1, ArCH₂N), 4.23 (d, 1, Ar₂CH, J=11.4 Hz), 3.78 (s, 3, OCH₃), 3.74 (s, 3, OCH₃), 3.61 (m, 1, CHN), 2.59 (m, 2, ArCH₂), 2.34 (s, 3, ArCH₃), 2.15 (m, 1, CH₂CN), 1.63 (m, 1, CH₂CN).

Trans-3-methyl-6-benzyl-10,11-dimethoxy-5,6,6a,7,8,12b-hexahydrobenzo[a]phenanthridine hydrochloride (4c). A solution of 0.773 g (1.872 mmol) of the lactam 3c, in 50 mL dry THF was cooled in an ice-salt bath and 4.0 equivalents (7.5 mL) of 1.0 molar BH₃ were added through a syringe. The reaction was heated at reflux under N₂ overnight. Methanol (6 mL) was added dropwise to the reaction mixture and reflux was continued for 1 hr. The solvent was removed. The residue was chased two times with methanol and twice with ethanol. The residue was placed under high vacuum (0.05 mm Hg) overnight. The residue was dissolved in ethanol and was carefully acidified with concentrated HCl. The volatiles were removed and the product was crystallized from ethanol to afford 0.652 g (80%) of 4c as the hydrochloride salt: mp 193-195° C.; CIMS (isobutane, M+1) 400; ¹H-NMR (CDCl₃, free base) δ 7.38 (d, 2, ArH), 7.33 (m, 2, ArH), 7.28 (m, 2, ArH), 7.07 (d, 1, ArH), 6.90 (s, 1, ArH), 6.88 (s, 1, ArH), 6.72 (s, 1, ArH), 4.02 (d, 1, Ar₂CH, J=11.2 Hz), 3.90 (d, 1, ArCH₂N), 3.87 (s, 3, OCH₃), 3.82 (m, 1, ArCH₂N), 3.78 (s, 3, OCH₃), 3.48 (d, 1, ArCH₂N), 3.30 (d, 1, ArCH₂N), 2.88 (m, 1, ArCH₂), 2.82 (m, 1, CHN), 2.36 (m, 1, CH₂CN), 2.32 (s, 3, ArCH₃), 2.20 (m, 1, ArCH₂), 1.95 (m, 1, CH₂CN).

Trans-3-methyl-10,11-dimethoxy-5,6,6a,7,8,12b-hexahydrobenzo[a]phenanthridine hydrochloride (5c). A solution of 0.643 g (1.47 mmol) of the 6-benzyl hydrochloride salt 4c prepared above in 100 mL of 95% ethanol containing 130 mg of 10% Pd/C catalyst was shaken at room temperature under 50 psig of H₂ for 8 hours. After removal of the catalyst by filtration through Celite, the solution was concentrated to dryness and the residue was recrystallized from acetonitrile to afford 0.397 g (78%) of 5c as a crystalline salt: mp 254-256° C.; CIMS (isobutane, M+1) 310; ¹H-NMR (DMSO, HCl salt) δ 10.01 (s, 1, NH), 7.36 (d, 1, ArH), 7.09 (d, 1, ArH), 6.98 (s, 1, ArH), 6.92 (s, 1, ArH), 6.74 (s, 1, ArH), 4.04 (s, 2, ArCH₂N), 3.88 (s, 3, OCH₃), 3.81 (s, 3, OCH₃), 3.76 (d, 1, Ar₂CH), 2.89 (m, 2, ArCH₂), 2.70 (m, 1, CHN), 2.36 (s, 3, ArCH₃), 2.16 (m, 1, CH₂CN), 1.70 (m, 1, CH₂CN).

Trans-3-methyl-10,11-dihydroxy-5,6,6a,7,8,12b-hexahydrobenzo[a]phenanthridine hydrochloride (6c). The 10,11-dimethoxy hydrochloride salt 5c (0.520 g, 1.51 mmol) was converted to its free base. The free base was dissolved in 35 mL of dichloromethane and the solution was cooled to −78° C. A 1.0 molar solution of BBr₃ (4.0 equivalents, 6.52 mL) was added slowly via syringe. The reaction was stirred under N₂ overnight with concomitant warming to room temperature. Methanol (7.0 mL) was added to the reaction mixture and the solvent was removed. The residue was placed under high vacuum (0.05 mm Hg) overnight. The residue was dissolved in water and was carefully neutralized to its free base initially with sodium bicarbonate and finally with ammonium hydroxide (1-2 drops). The free base was isolated by suction filtration and was washed with cold water. The filtrate was extracted several times with dichloromethane and the organic extracts were dried, filtered, and concentrated. The filter cake and the organic residue were combined, dissolved in ethanol, and carefully acidified with concentrated HCl. After removal of the volatiles, the HCl salt was crystallized as a solvate from methanol to yield 0.341 g (71.3%) or 6c: mp 190° C. (dec.); CIMS (isobutane, M+1) 282; ¹H-NMR (DMSO, HCl salt) δ 9.55 (s, 1, NH), 8.85 (d, 2, OH), 7.30 (d, 1, ArH), 7.22 (s, 1, ArH), 7.20 (d, 1, ArH), 6.68 (s, 1, ArH), 6.60 (s, 1, ArH), 4.31 (s, 2, ArCH₂N), 4.09 (d, 1, ArCH₂CH, J=11.2 Hz), 2.91 (m, 1, CHN), 2.72 (m, 2, ArCH₂), 2.35 (s, 3, ArCH₃), 2.16 (m, 1, CH₂CN, 1.85 (m, 1, CH₂CN).

### Example 4

### 4-Methyldihydrexidine (6d)

2-(N-benzyl-N-2-methylbenzoyl)-6,7-dimethoxy-3,4-dihydro-2-naphthylamine (2d). To a solution of 5.123 g (24.8 mmol) of 6,7-dimethoxy-β-tetralone in 200 mL of toluene was added 2.929 g (1.025 equivalents) of benzylamine. The reaction was heated at reflux overnight under N₂ with continuous water removal. The reaction was cooled and the solvent was removed to yield the N-benzyl enamine as a brown oil.

The 2-methylbenzoyl chloride acylating agent was prepared by suspending 4.750 g (42.2 nmol) of 2-toluic acid in 100 mL benzene. To this solution was added 2.0 equivalents (7.37 mL) of oxalyl chloride, dropwise via a pressure-equalizing dropping funnel at 0° C. Catalytic DMF (2-3 drops) was added to the reaction mixture and the ice bath was removed. The progress of the reaction was monitored using infrared spectroscopy. The solvent was removed and the residual oil was held under high vacuum overnight.

The resulting N-benzyl enamine residue was dissolved in 100 mL of CH₂Cl₂, and to this solution was added 2.765 g (1.1 equivalent) of triethylamine at O C. The 2-methylbenzoyl chloride (4.226 g, 27.3 mmol) was dissolved in 25 mL CH₂Cl₂ and this solution was added dropwise to the cold, stirring N-benzyl enamine solution. The reaction was allowed to warm to room temperature and was left to stir under N₂ overnight. The reaction mixture was washed successively with 2×30 mL of 5% aqueous HCl, 2×30 mL of saturated sodium bicarbonate solution, saturated NaCl solution, and was dried over MgSO₄. After filtration, the filtrate was concentrated. The resulting oil was purified via a chromatotron utilizing a 5% ether/dichloromethane eluent mobile phase to yield 3.950 g (38.5%) of 2d as an oil: CIMS (isobutane, M+1) 414; ¹H-NMR (CDCl₃) δ 7.34 (d, 2, ArH), 7.30 (m, 2, ArH), 7.25 (d, 2, ArH), 7.14 (m, 2, ArH), 7.07 (m, 1, ArH), 6.47 (s, 1, ArH), 6.37 (s, 1, ArH), 6.04 (s, 1, ArCH), 4.96 (s, 2, ArCH₂N), 3.78 (s, 3, OCH₃), 3.77 (s, 3, OCH₃), 2.39 (s, 3, ArCH₃), 2.30 (t, 2, ArCH₂), 1.94 (t, 2, CH₂).

Trans-4-methyl-6-benzyl-10,11-dimethoxy-5,6,6a,7,8,12b-hexahydrobenzo[a]phenanthridine-5-one (3d). A solution of 2.641 g (6.395 mmol) of the 6,7-dimethoxyenamide 2d, in 450 mL of THF, was introduced to an Ace Glass 500 mL photochemical reactor. This solution was stirred while irradiating for 3 hours with a 450 watt Hanovia medium pressure, quartz, mercury-vapor lamp seated in a water-cooled, quartz immersion well. The solution was concentrated and crystallized from diethyl ether to provide 0.368 (20%) of the 10,11-dimethoxy lactam 3d: mp 175-176° C.; CIMS (isobutane, M+1) 414; ¹H-NMR (CDCl₃) δ 7.88 (m, 3, ArH), 7.65 (d, 1, ArH), 7.40 (m, 2, ArH), 7.21 (m, 2, ArH), 6.87 (s, 1, ArH), 6.60 (s, 1, ArH), 5.34 (d, 1, ArCH₂N), 4.72 (d, 1, ArCH₂N), 4.24 (d, 1, Ar₂CH, J=10.9 Hz), 3.86 (s, 3, OCH₃), 3.85 (s, 3, OCH₃), 3.68 (m, 1, CHN), 2.73 (s, 3, ArCH₃), 2.64 (m, 2, ArCH₂); 2.20 (m, 1, CH₂CN), 1.72 (m, 1, CH₂CN).

Trans-4-methyl-6-benzyl-10,11-dimethoxy-5,6,6a,7,8,12b-hexahydrobenzo[a]phenanthridine hydrochloride (4d). A solution of 1.640 g (3.97 mmol) of the lactam 3d, in 100 mL dry THF was cooled in an ice-salt bath and 4.0 equivalents (15.9 mL) of 1.0 molar BH₃ were added through a syringe. The reaction was heated at reflux under N₂ overnight. Methanol (10 mL) was added dropwise to the reaction mixture and reflux was continued for 1 hour. The solvent was removed and the residue was chased two times with methanol and twice with ethanol. The residue was placed under high vacuum (0.05 mm Hg) overnight. The residue was dissolved in ethanol and was carefully acidified with concentrated HCl. The volatiles were removed and the product was crystallized from ethanol to afford 1.288 g (74.5%) of 4d as the hydrochloride salt: mp 232-235° C.; CIMS (isobutane, M+1), 400; ¹H-NMR (CDCl₃, free base) δ 7.38 (d, 2, ArH), 7.33 (m, 2, ArH), 7.27 (d, 1, ArH), 7.24 (m, 1, ArH), 7.16 (m, 1, ArH), 7.06 (d, 1, ArH), 6.85 (s, 1, ArH), 6.71 (s, 1, ArH), 4.05 (d, 1, Ar₂CH, J=10.8 Hz), 3.89 (d, 1, ArCH₂N), 3.87 (s, 3, OCH₃), 3.82 (m, 1, ArCH₂N), 3.76 (s, 3, OCH₃), 3.55 (d, 1, ArCH₂N), 3.31 (d, 1, ArCH₂N), 2.88 (m, 1, ArCH₂), 2.81 (m, 1, CHN), 2.34 (m, 1, CH₂CN), 2.20 (m, 1, ArCH₂), 2.17 (s, 3, ArCH₃), 1.94 (m, 1, CH₂CN).

Trans-4-methyl-10,11-dimethoxy-5,6,6a,7,8,12b-hexahydrobenzo[a]phenanthridine hydrochloride (5d). A solution of 0.401 g (0.92 mmol) of the 6-benzyl hydrochloride salt 4d in 100 mL of 95% ethanol containing 100 mg of 10% Pd/C catalyst was shaken at room temperature under 50 psig of H₂ for 8 hours. After removal of the catalyst by filtration through Celite, the solution was concentrated to dryness and the residue was recrystallized from acetonitrile to afford 0.287 g (90.2%) of 5d as a crystalline salt: mp 215-216° C.; CIMS (isobutane, M+1) 310; ¹H-NMR (CDCl₃, free base) δ 9.75 (s, 1, NH), 7.29 (d, 1, ArH), 7.28 (d, 1, ArH), 7.21 (m, 1, ArH), 6.86 (s, 1, ArH), 6.81 (s, 1, ArH), 4.35 (d, 1, ArCH₂N), 4.26 (d, 1, ArCH₂N), 4.23 (d, 1, Ar₂CH, J=11.17 Hz), 3.75 (s, 3, OCH₃), 3.65 (s, 3, OCH₃), 2.96 (m, 1, CHN), 2.83 (m, 2, ArCH₂), 2.30 (s, 3, ArCH₃), 2.21 (m, 1, CH₂CN), 1.93 (m, 1, CH₂CN).

Trans-4-methyl-10,11-dihydroxy-5,6,6a,7,8,12b-hexahydrobenzo[a]phenanthridine hydrochloride (6d). The 10,11-dimethoxy hydrochloride salt 5d (0.485 g, 1.40 mmol) was converted to its free base. The free base was dissolved in 35 mL of dichloromethane and the solution was cooled to −78° C. A 1.0 molar solution of BBr₃ (4.0 equivalents, 5.52 mL) was added slowly through a syringe. The reaction was stirred under N₂ overnight with concomitant warming to room temperature. Methanol (7.0 mL) was added to the reaction mixture and the solvent was removed. The residue was placed under high vacuum (0.05 mm Hg) overnight. The residue was dissolved in water and was carefully neutralized to its free base initially with sodium bicarbonate and finally with ammonium hydroxide (1-2 drops). The free base was isolated by suction filtration and was washed with cold water, the filtrate was extracted several times with dichloromethane and the organic extracts were dried, filtered, and concentrated. The filter cake and the organic residue were combined, dissolved in ethanol and carefully acidified with concentrated HCl. After removal of the volatiles, the HCl salt was crystallized as a solvate from methanol to yield 0.364 g (81.6%) of 6d: mp 195° C. (dec.); CIMS (isobutane, M+1) 282; ¹H-NMR (DMSO, HCl salt) d 9.55 (s, 1, NH), 8.85 (s, 1, OH), 8.80 (s, 1, OH), 7.28 (m, 2, ArH), 7.20 (d, 1, ArH), 6.65 (s, 1, ArH), 6.60 (s, 1, ArH), 4.32 (d, 1, ArCH₂N), 4.26 (d, 1, ArCH₂N), 4.13 (d, 1, Ar₂CH, J=11.63 Hz), 2.92 (m, 1, CHN), 2.75 (m, 1, ArCH₂), 2.68 (m, 1, ArCH₂), 2.29 (s, 3, ArCH₃), 2.17 (m, 1, CH₂CN), 1.87 (m, 1, CH₂CN).

### Example 5

### 2-Benzyldihydrexidine (6e)

Trans-2-benzyl-10,11-dihydroxy-5,6,6a,7,8,12b-hexahydrobenzo[a]phenanthridine hydrochloride (6e) prepared according to the procedure described in Example 4, except that 4-methylbenzoyl chloride was replaced with 2-benzylbenzoyl chloride.

### Example 6

### Dinoxyline (16a)

1,2-Dimethoxy-3-methoxymethoxybenzene (8). A slurry of sodium hydride was prepared by adding 1000 mL of dry THF to 7.06 g (0.18 mol) of sodium hydride (60% dispersion in mineral oil) under an argon atmosphere at 0° C. To the slurry, 2,3-dimethoxyphenol (7) (23.64 g, 0.153 mol) was added through a syringe. The resulting solution was allowed to warm to room temperature and stirred for two hours. The resulting black solution was cooled to 0° C. and 13.2 mL of chloromethylmethyl ether (14 g, 0.173 mol) was slowly added with a syringe. The solution was allowed to reach room temperature and stirred for an additional 8 hours. The resulting yellow mixture was concentrated to an oil that was dissolved in 1000 mL of diethyl ether. The resulting solution was washed with water (500 mL), 2N NaOH (3×400 mL), dried (MgSO₄), filtered, and concentrated. After Kugelrohr distillation (90-100° C., 0.3 atm), 24.6 g (84%) of 8 as a clear oil was obtained: ¹H NMR (300 MHz, CDCl₃) δ 6.97 (t, 1H, J=8.7 Hz); 6.79 (dd, 1H, J=7.2, 1.8 Hz); 6.62 (dd, 1H, J=6.9, 1.2 Hz); 5.21 (s, 2H); 3.87 (s, 3H); 3.85 (s, 3H); 3.51 (s, 3H); CIMS m/z 199 (M+H⁺, 50%); 167 (M+H⁺, CH₃OH, 100%); Anal. Calc'd for C₁₀H₁₄O₄: C, 60.59; H, 7.12. Found: C, 60.93; H, 7.16.

2-(3,4-Dimethoxy-2-methoxymethoxyphenyl)-4,4,5,5-tetra-methyl-1,3,2-dioxaborolane (9). The MOM-protected phenol 8 (10 g, 0.0505 mol) was dissolved in 1000 mL of dry diethyl ether and cooled to −78° C. A solution of n-butyl lithium (22.2 mL, 2.5 M) was then added with a syringe. The cooling bath was removed and the solution was allowed to warm to room temperature. After stirring the solution at room temperature for two hours, a yellow precipitate was observed. The mixture was cooled to −78° C., and 15 mL of 2-isopropoxy-4,4,5,5-tetramethyl-1,3,2-dioxaborolane (0.080 mol) was added through a syringe. The cooling bath was removed after two hours. Stirring was continued for four hours at room temperature. The mixture was then poured into 300 mL of water and extracted several times with diethyl ether (3×300 mL), dried (Na₂SO₄), and concentrated to 9 a yellow oil (12.37 g, 76%) that was used without further purification: ¹H NMR (300 MHz, CDCl₃) δ 7.46 (d, 1H, J=8.4 Hz); 6.69 (d, 1H, J=8.4 Hz); 5.15 (s, 2H); 3.87 (s, 3H); 3.83 (s, 3H); 1.327 (s, 12H).

4-Bromo-5-nitroisoquinoline (11). Potassium nitrate (5.34 g; 0.052 mol) was added to 20 mL of concentrated sulfuric acid and slowly dissolved by careful heating. The resulting solution was added dropwise to a solution of 4-bromoisoquinoline (10 g, 0.048 mol) dissolved in 40 mL of the same acid at 0° C. After removal of the cooling bath, the solution was stirred for one hour at room temperature. The reaction mixture was then poured onto crushed ice (400 g) and made basic with ammonium hydroxide. The resulting yellow precipitate was collected by filtration and the filtrate was extracted with diethyl ether (3×500 μL), dried (Na₂SO₄), and concentrated to give a yellow solid that was combined with the initial precipitate. Recrystallization from methanol gave 12.1 g (89%) of 11 as slightly yellow crystals: mp 172-174° C.; ¹H NMR (300 MHz, CDCl₃) δ 9.27 (s, 1H); 8.87 (s, 1H); 8.21 (dd, 1H, J=6.6, 1.2 Hz); 7.96 (dd, 1H, J=6.6, 1.2 Hz); 7.73 (t, 1H, J=7.5 Hz); CIMS m/z 253 (M+H⁺, 100%); 255 (M+H⁺+2, 100%); Anal. Calc'd for C₉H₅BrN₂O₂: C, 42.72; H, 1.99; N, 11.07. Found: C, 42.59; H, 1.76; N, 10.87.

4-(3,4-Dimethoxy-2-methoxymethoxyphenyl)-5-nitroisoquinoline (12). Isoquinoline 11 (3.36 g, 0.0143 mol), pinacol boronate ester 9 (5.562 g, 0.0172 mol), and 1.0 g (6 mol %) of (Ph₃)Pd were suspended in 100 mL of dimethoxyethane (DME). Potassium hydroxide (3.6 g; 0.064 mol), and 0.46 g (10 mol %) of tetrabutylammonium bromide were dissolved in 14.5 mL of water and added to the DME mixture. The resulting suspension was degassed for 30 minutes with argon and then heated at reflux for four hours. The resulting black solution was allowed to cool to room temperature, poured into 500 mL of water, extracted with diethyl ether (3×500 mL), dried (Na₂SO₄), and concentrated. The product was then purified by column chromatography (silica gel, 50% ethyl acetate-hexane) giving 5.29 g (80.1%) of 12 as yellow crystals: mp 138-140° C.; ¹H NMR (300 MHz, CDCl₃) δ 9.33 (s, 1H); 8.61 (s, 1H); 8.24 (dd, 1H, J=7.2, 0.9 Hz); 8.0 (dd, 1H, J=6.3, 1.2 Hz); 7.67 (t, 1H, J=7.8 Hz); 7.03 (d, 1H, J=9.6 Hz); 6.81 (d, 1H, J=8.1 Hz); 4.86 (d, 1H, J=6 Hz); 4.70 (d, 1H, J=5.4 Hz); 3.92 (s, 3H); 3.89 (s, 3H); 2.613 (s, 3H); CIMS m/z 371 (M+H⁺, 100%); Anal. Calc'd for C₁₉H₁₈N₂O₆: C, 61.62; H, 4.90; N, 7.56. Found: C, 61.66; H, 4.90; N, 7.56.

2,3-Dimethoxy-6-(5-nitroisoquinolin-4-yl)phenol (13). After dissolving isoquinoline 12 (5.285 g, 0.014 mol) in 200 mL of methanol by gentle heating, p-toluenesulfonic acid monohydrate (8.15 g; 0.043 mol) was added in several portions. Stirring was continued for four hours at room temperature. After completion of the reaction, the solution was made basic by adding saturated sodium bicarbonate. The product was then extracted with CH₂Cl₂ (3×250 mL), dried (Na₂SO₄), and concentrated. The resulting 13 as a yellow solid (4.65 g; 98%) was used directly in the next reaction. An analytical sample was recrystallized from methanol: mp 170-174° C.; ¹H NMR (300 MHz, CDCl₃) δ 9.33 (s, 1H); 8.62 (s, 1H); 8.24 (dd, 1H, J=7.2, 0.9 Hz); 7.99 (dd, 1H, J=6.3, 1.2 Hz); 7.67 (t, 1H, J=7.8 Hz); 6.96 (d, 1H, J=8.7 Hz); 6.59 (d, 1H, J=8.7 Hz); 5.88 (bs, 1H); 3.94 (s, 3H); 3.92 (s, 3H); CIMS m/z 327 (M+H⁺, 100%); Anal. Calc'd for C₁₇H₁₄N₂O₅: C, 62.57; H, 4.32; N, 8.58; Found: C, 62.18; H, 4.38; N, 8.35.

8,9-dimethoxychromeno[4,3,2-de]isoquinoline (14). Phenol 13 (4.65 g, 0.014 mol) was dissolved in 100 mL of dry DMF. The solution was degassed with argon for thirty minutes. Potassium carbonate (5.80 g, 0.042 mol) was added to the yellow solution in one portion. After heating at 80° C. for one hour, the mixture had turned brown and no more starting material remained. After the solution was cooled to room temperature, 200 mL of water was added. The aqueous layer was extracted with dichloromethane (3×500 mL), this organic extract was washed with water (3×500 mL), dried (Na₂SO₄), and concentrated. Isoquinoline 14 was obtained as a white powder (3.65 g 92%) and was used in the next reaction without further purification. An analytical sample was recrystallized from ethyl acetate:hexane: mp 195-196° C.; ¹H NMR (300 MHz, CDCl₃) δ 9.02 (s, 1H); 8.82 (s, 1H); 7.87 (d, 1H, J=8.7 Hz); 7.62 (m, 3H); 7.32 (dd, 1H, J=6.0, 1.5 Hz); 6.95 (d, J=9.6 Hz); 3.88 (s, 3H); 3.82 (s, 3H). CIMS ml z 280 (M+H⁺, 100%).

8,9-dimethoxy-1,2,3,11b-tetrahydrochromeno[4,3,2-de]isoquinoline (15a). Platinum (IV) oxide (200 mg) was added to a solution containing 50 mL of acetic acid and isoquinoline 14 (1 g; 3.5 mmol). After adding 2.8 mL of concentrated HCl, the mixture was shaken on a Parr hydrogenator at 60 psi for 24 hours. The resulting green solution was filtered through Celite to remove the catalyst and the majority of the acetic acid was removed under reduced pressure. The remaining acid was neutralized using a saturated sodium bicarbonate solution, extracted with diethyl ether (3×250 mL), dried (Na₂SO₄), and concentrated. The resulting 14 as an oil (0.997 g; 99%) was used without further purification: ¹H NMR (300 MHz, CDCl₃) δ 7.10 (t, 1H, J=7.5 Hz); 7.00 (d, 1H, J=8.4 Hz); 6.78 (m, 2H); 6.60 (d, 1H, J=9 Hz); 4.10 (s, 2H); 3.84 (m, 8H); 2.93 (t, 1H, J=12.9 Hz).

8,9-dihydroxy-1,2,3,11b-tetrahydrochromeno[4,3,2-de]isoquinoline hydrobromide (16a). The dimethoxy derivative 15a (0.834 g; 3.0 mmol) was dissolved in 50 mL of anhydrous dichloromethane. The solution was cooled to −78° C. and 15.0 mL of a boron tribromide solution (1.0 M in dichloromethane) was slowly added. The solution was stirred overnight, while the reaction slowly warmed to room temperature. The solution was recooled to −78° C., and 50 mL of methanol was slowly added to quench the reaction. The solution was then concentrated to dryness. Methanol was added and the solution was concentrated. This process was repeated three times. The resulting brown solid was treated with activated charcoal and recrystallized from ethanol to give 16a: mp 298-302° C. (dec.); ¹H NMR (300 MHz, D₂O) δ 7.32 (t, 1H, J=6.6 Hz); 7.13 (d, 1H, J=8.4 Hz); 7.04 (d, 1H, J=8.4 Hz); 4.37 (m, 2H); 4.20 (t, 3H, J=10 Hz); Anal. Calc'd for C₁₅H₁₄BrNO₃.H₂O: C, 50.87; H, 4.55; N, 3.82. Found: C, 51.18; H, 4.31; N, 3.95.

### Example 7

### N-Allyl dinoxyline (16b)

N-allyl-8,9-dimethoxy-1,2,3,11b-tetrahydrochromeno[4,3,2-de]isoquinoline (15b). Tetrahydroisoquinoline 15a (1.273 g; 4.5 mmol) was dissolved in 150 mL of acetone. Potassium carbonate (0.613 g; 4.5 mmol) and 0.4 mL (4.6 mmol) of allyl bromide were added. The reaction was stirred at room temperature for four hours. The solid was then removed by filtration and washed on the filter several times with ether. The filtrate was concentrated and purified by flash chromatography (silica gel, 50% ethyl acetate-hexane) to give 1.033 g (71%) of 15b a yellow oil that was used without further purification: ¹H NMR (300 MHz, CDCl₃) δ 7.15 (t, 1H, J=9 Hz); 7.04 (d, 1H, J=9 Hz); 6.83 (m, 2H); 6.65 (d, 1H, J=6 Hz); 5.98 (m, 1H); 5.27 (m, 2H); 4.10 (m, 3H); 3.95 (s, 3H); 3.86 (s, 3H); 3.46 (d, 1H, J=15 Hz); 3.30 (d, 2H, J=6 Hz); 2.56 (t, 1H, J=12 Hz).

N-allyl-8,9-dihydroxy-1,2,3,11b-tetrahydrochromeno[4,3,2-de]isoquinoline (16b). N-Allylamine 15b (0.625 g; 1.93 mmol) was dissolved in 50 mL of dichloromethane. The solution was cooled to −78° C. and 10.0 mL of BBr₃ solution (1.0 M in dichloromethane) was slowly added. The solution was stirred overnight, while the reaction slowly warmed to room temperature. After recooling the solution to −78° C., 50 mL of methanol was slowly added to quench the reaction. The reaction was then concentrated to dryness. Methanol was added and the solution was concentrated. This process was repeated three times. Recystallization of the resulting brown solid from ethanol gave 0.68 g (61%) of 16b as a white solid: mp 251-253° C. (dec.); ¹H NMR (300 MHz, D₂O) δ 10.55 (s, 1H); 10.16 (s, 1H); 8.61 (t, 1H, J=9 Hz); 8.42 (d, 1H, J=9 Hz); 8.31 (d, 1H, J=9 Hz); 7.87 (d, 1H, J=9 Hz); 7.82 (d, 1H, J=9 Hz); 7.36 (q, 1H, J=9 Hz); 6.89 (m, 2H); 6.85 (d, 1H, J=15 Hz); 5.58 (m, 3H); 5.28 (m, 2H); 3.76 (d, 1H, J=3 Hz). HRCIMS m/z Calc'd: 295.1208;

Found: 295.1214.

### Example 8

### N-Propyl dinoxyline (16c)

N-propyl-8,9-dimethoxy-1,2,3,11b-tetrahydrochromeno[4,3,2-de]isoquinoline (15c). N-Allylamine 15b (1.033 g; 3.2 mmol) was dissolved in 50 mL of ethanol. Palladium on charcoal (10% dry; 0.103 g) was then added. The mixture was shaken on a Parr hydrogenator under 60 psi H₂ for 3 hours. After TLC showed no more starting material, the mixture was filtered through Celite and concentrated to give 0.95 g (91%) of 15c as an oil that was used without further purification: ¹H NMR (300 MHz, CDCl₃) δ 7.15 (t, 1H, J=7.2 Hz); 7.04 (d, 1H, J=8.1 Hz); 6.84 (t, 2H, J=7.5 Hz); 6.65 (d, 1H, J=8.4 Hz); 4.07 (m, 2H); 3.95 (s, 3H); 3.86 (s, 3H); 3.71 (q, 1H, J=5.1 Hz); 3.42 (d, 2H, J=15.6 Hz); 2.62 (m, 2H); 2.471 (t, J=10.5 Hz); 1.69 (h, 2H, J=7.2 Hz); 0.98 (t, 3H, J=7.5 Hz); CIMS m/z 326 (M+W⁺, 100%).

N-propyl-8,9-dihydroxy-1,2,3,11b-tetrahydrochromeno[4,3,2-de]isoquinoline (16c). The N-propyl amine 15c (0.90 g; 2.8 mmol) was dissolved in 200 mL of dichloromethane and cooled to −78° C. In a separate 250 mL round bottom flask, 125 mL of dry dichloromethane was cooled to −78° C., and 1.4 mL (14.8 mmol) of BBr₃ was added through a syringe. The BBr₃ solution was transferred using a cannula to the flask containing the starting material. The solution was stirred overnight, while the reaction slowly warmed to room temperature. After recooling the solution to −78° C., 50 mL of methanol was slowly added to quench the reaction. The reaction was then concentrated to dryness. Methanol was added and the solution was concentrated. This process was repeated three times. The resulting tan solid was suspended in hot isopropyl alcohol. Slowly cooling to room temperature resulted in a fine yellow precipitate. The solid was collected by filtration to give 16c (0.660 g; 63%): mp 259-264° C. (dec.); ¹H NMR (300 MHz, CDCl₃) δ 7.16 (t, 1H, J=9 Hz); 6.97 (d, 1H, J=12 Hz); 6.83 (d, 1H, i=9 Hz); 6.55 (d, 1H, J=9 Hz); 6.46 (d, 1H, J=9 Hz); 4.45 (d, 1H, J=15 Hz); 4.10 (m, 3H); 3.17 (q, 2H, J=6 Hz); 3.04 (t, 1H, =9 Hz); 1.73 (q, 2H, J=9 Hz); 0.90 (t, 3H, J=6 Hz); Anal. Calc'd. for C₁₈H₂₀BrNO₃: C, 57.16; H, 5.33; N, 3.70. Found: C, 56.78; H, 5.26; N, 3.65.

### Example 9

### Preparation of 2-methyl-2,3-dihydro-4(1H)-isoquinolone (20)

Ethyl 2-bromomethylbenzoate (18). A solution of ethyl 2-toluate (17, 41.2 g, 0.25 mole) in carbon tetrachloride (200 mL) was added dropwise to a stirring mixture of benzoyl peroxide (100 mg), carbon tetrachloride (200 mL), and NBS (44.5 g, 0.25 mole) at 0° C. The mixture was heated at reflux for 3.5 hr under nitrogen, and allowed to cool to room temperature overnight. The precipitated succinimide was removed by filtration and the filter cake was washed with carbon tetrachloride. The combined filtrates were washed successively with 2 N NaOH (100 mL), and water (2×100 mL), and the solution was dried over anhydrous MgSO₄, filtered (Celite), and evaporated under vacuum to yield an oil. Drying under high vacuum overnight afforded 60.5 g (99%) of compound 18: ¹H NMR of the product showed the presence of ca. 15% of unreacted 17. The mixture was used in the next step without further purification: ¹H NMR (CDCl₃) δ 1.43 (t, J=7 Hz, 3H, CH₂ CH₃), 4.41 (q, J=7 Hz, 2H, CH₂CH₃), 4.96 (s, 1H, CH₂Br), 7.24 (m, 1H, ArH), 7.38 (m, 1H, ArH), 7.48 (m, 2H, ArH).

N-(2-carboethoxy)sarcosine ethyl ester (19). To a mixture of sarcosine ethyl ester hydrochloride (32.2 g, 0.21 mole), potassium carbonate (325 mesh; 86.9 g, 0.63 mole), and acetone (800 mL) was added a solution of compound 18 (60.7 g, ca. 0.21 mole, 85:15 18/17) in acetone (100 mL) at room temperature under N₂. The mixture was stirred at reflux for 2 hr and then left at room temperature for 20 hr. The solid was removed by filtration (Celite) and the residue was washed with acetone. The filtrates were combined and evaporated to afford an oil. The oil was dissolved in 250 mL of 3 N HCl and washed with ether. The aqueous layer was basified with aqueous NaHCO₃, and extracted with ether (3×250 mL). Evaporation of the ether solution yielded an oil that was vacuum distilled to afford 45.33 g (77%) of compound 19: bp 140-142° C./0.5 mm Hg; bp 182-183° C./10 mm Hg; ¹HNMR (CDCl₃) δ 1.24 (t, 3H, J=7.1 Hz, CH₃), 1.36 (t, 3H, J=7.1 Hz, CH₃), 2.35 (s, 3H, NCH₃), 3.27 (s, 2H, CH₂Ar), 4.00 (s, 2H, NCH₂), 4.14 (q, 2H, J=7.1 Hz, CH₂CH₃), 4.32 (q, 2H, J=7.1 Hz, CH₂CH₃), 7.28 (t, 1H, J=7.4 Hz, ArH), 7.42 (t, 1H, J=7.6 Hz, ArH), 7.52 (d, 1H, J=7.8 Hz, ArH), 7.74 (d, 1H, J=7.7 Hz, ArH).

2-Methyl-2,3-dihydro-4(1H)isoquinolone (20). Freshly cut sodium (10.9 g, 0.47 g-atom) was added to absolute ethanol (110 mL) under nitrogen and the reaction was heated at reflux. After the metallic sodium had disappeared, a solution of compound 19 (35.9 g, 0.128 mole) in dry toluene (160 mL) was added slowly to the reaction mixture. It was then heated at reflux and ethanol was separated azeotropically via a Dean Stark trap. After cooling, the solvent was evaporated under reduced pressure. The remaining yellow semi-solid residue was dissolved in a mixture of water (50 mL), 95% ethanol (60 mL), and concentrated HCl (240 mL), and heated at reflux for 26 hr. After cooling, the mixture was concentrated under vacuum and carefully basified with solid NaHCO₃. The basic solution was extracted with ether, dried (MgSO₄), and evaporated to an oil that was distilled to afford compound 20 (17.11 g, 83%): bp 130-132° C./5 mm Hg; bp 81-83° C./0.4 mm Hg; mp (HCl salt) 250° C.; IR (neat) 1694 (C═O) cm⁻¹; ¹HNMR (CDCl₃) δ 2.48 (s, 3H, CH₃), 3.31 (s, 2H, CH₂), 3.74 (s, 2H, CH₂), 7.22 (d, 1H, J=7.7 Hz, ArH), 7.34 (t, 1H, J=7.9 Hz, ArH), 7.50 (t, 1H, J=7.5 Hz, ArH), 8.02 (d, 1H, J=7.9 Hz, ArH).

### Example 10

### Dinapsoline (29)

2′,3′-Dihydro-4,5-dimethoxy-2′-methylspiro[isobenzofuran-1(3H), 4′(1′H)-isoquinoline]-3-one (22). To a solution of 2,3-dimethoxy-N,N′-diethylbenzamide (21, 14.94 g, 63 mmol) in ether (1400 mL) at −78° C. under nitrogen was added sequentially, dropwise, N,N,N′,N′-tetramethylenediamine (TMEDA, 9.45 mL, 63 mmol), and sec-butyllithium (53.3 mL, 69 mmol, 1.3 M solution in hexane). After 1 hr, freshly distilled compound 20 (10.1 g, 62.7 mmol) was added to the heterogenous mixture. The cooling bath was removed and the reaction mixture was allowed to warm to room temperature over 9 hr. Saturated NH₄Cl solution (400 mL) was then added and the mixture was stirred for 15 min. The ether layer was separated and the water layer was extracted with dichloromethane (4×100 mL). The organic layers were combined, dried (MgSO₄), and evaporated to a brown oil. The oil was dissolved in toluene (500 mL), and heated at reflux for 8 hr with 3.0 g of p-toluene sulfonic acid, cooled, and concentrated under vacuum. The residue was dissolved in dichloromethane, washed with dilute aqueous NaHCO₃, water, and then dried (Na₂SO₄), filtered, and evaporated to a gummy residue. On trituration with ethyl acetate/hexane (50:50), a solid precipitated. Recrystallization from ethyl acetate/hexane afforded 12.75 g (63%) of compound 22: mp 193-194° C.; IR (KBr) 1752 cm⁻¹ (C═O); ¹H NMR (CDCl₃) δ 2.47 (s, 3H, NCH₃), 2.88 (d, 1H, J=11.6 Hz), 3.02 (d, 1H, J=11.7 Hz), 3.76 (d, 1H, J=15.0 Hz), 3.79 (d, 1H, J=15.1 Hz), 3.90 (s, 3H, OCH₃), 4.17 (s, 3H, OCH₃), 6.83 (d, 1H, J=8.4 Hz, ArH), 7.03 (d, 1H, J=8.2 Hz, ArH), 7.11 (m, 3H, ArH), 7.22 (m, 1H, ArH); MS (CI) m/z 326 (100).

2′,3′-Dihydro-4,5-dimethoxyspiro[isobenzofuran-1(3H), 4′(1′H)-isoquinoline]-3-one (23). 1-chloroethyl chloroformate (5.1 mL, 46.3 mmol) was added dropwise to a suspension of compound 22 (6.21 g, 19.2 mmol) in 100 mL of 1,2-dichloroethane at 0° C. under nitrogen. The mixture was stirred for 15 min at 0° C., and then heated at reflux for 8 hr. The mixture was cooled, and concentrated under reduced pressure. To this mixture was added 75 mL of methanol and the reaction was heated at reflux overnight. After cooling, the solvent was evaporated to afford the hydrochloride salt of compound 23 in nearly quantitative yield. It was used in the next step without further purification: mp (HCl salt) 220-222° C.; mp (free base) 208-210° C.; IR (CH₂Cl₂, free base) 1754 cm⁻¹ (C═O); ¹H NMR (CDCl₃, free base) δ 3.18 (d, 1H, J=13.5 Hz), 3.30 (d, 1H, J=13.5 Hz), 3.84 (s, 3H, OCH₃), 3.96 (s, 3H, OCH₃), 4.02 (s, 2H, CH₂N), 6.67 (d, 1H, J=7.5 Hz, ArH), 7.12 (m, 2H, ArH), 7.19 (d, 1H, J=7.5 Hz, ArH), 7.26 (t, 1H, J=7.5 Hz, ArH), 7.41 (d, 1H, J=8.5 Hz, ArH); MS (CI) m/z 312 (100); HRCIMS Calc'd for C₁₈H₁₇NO₄: 312.1236; Found 312.1198; Anal. Calc'd for C₁₈H₁₇NO₄: C, 69.44. Found: C, 68.01.

2′,3′-Dihydro-4,5-dimethoxy-2′-p-toluenesulfonylspiro[isobenzofuran-1(3H), 4′(1′H)isoquinoline]-3-one (24). Triethylamine (7 mL) was added dropwise to a mixture of p-toluenesulfonyl chloride (3.6 g, 18.9 mmole), compound 23 (as the HCl salt, obtained from 19.2 mmol of compound 22), and chloroform (100 mL) at 0 C under nitrogen. After the addition was complete, the ice bath was removed and the reaction mixture was stirred at room temperature for 1 hr. It was then acidified with 100 mL of cold aqueous 0.1 N HCl, extracted with dichloromethane (2×100 mL), and the organic extract was dried (MgSO₄), filtered, and evaporated to afford a viscous liquid that on trituration with ethyl acetate/hexane at 0° C. gave a solid. Recrystallization from ethyl acetate/hexane afforded 8.74 g (97%, overall from compound 22) of compound 24: mp 208-210° C.; IR (KBr) 1767 cm⁻¹ (C═O); ¹H NMR (CDCl₃) δ 2.43 (s, 1H, CH₃), 3.22 (d, 1H, J=11 Hz), 3.88 (d, 1H, J=11 Hz), 3.90 (s, 3H, OCH₃), 3.96 (d, 1H, J=15 Hz), 4.17 (s, 3H, OCH₃), 4.81 (d, 1H, J=15 Hz), 6.97 (d, 1H, J=7.7 Hz, ArH), 7.16 (m, 3H, ArH), 7.26 (m, 1H, ArH), 7.38 (d, 2H, J=8 Hz, ArH), 7.72 (d, 2H, J=8 Hz, ArH); MS (CI) m/z 466 (100).

3,4-Dimethoxy-6-[(2-p-toluenesulfonyl-1,2,3,4-tetrahydroisoquinoline)-4-yl]benzoic acid (25). A solution of compound 24 (2.56 g, 5.51 mmol) in glacial acetic acid (250 mL) with 10% palladium on activated carbon (6.30 g) was shaken on a Parr hydrogenator at 50 psig for 48 hr at room temperature. The catalyst was removed by filtration, and the solvent was evaporated to afford 2.55 g (99%) of compound 25. An analytical sample was recrystallized from ethanol/water: mp 182-184° C.; IR (KBr) 1717 cm⁻¹ (COOH); ¹H NMR (DMSO-d₆) δ 2.35 (s, 3H, CH₃), 3.12 (m, 1H), 3.51 (dd, 1H, J=5, 11.5 Hz), 3.71 (s, 6H, OCH₃), 4.10 (m, 1H, Ar₂CH), 4.23 (s, 2H, ArCH₂N), 6.52 (d, 1H, J=7.5 Hz, ArH), 6.78 (d, 1H, J=7.5 Hz, ArH), 6.90 (m, 1H, ArH), 7.07 (t, 1H, J=8 Hz, ArH), 7.14 (t, 1H, J=6.5 Hz, ArH), 7.20 (d, 1H, J=7.5 Hz, ArH), 7.38 (d, 2H, J=8 Hz, ArH), 7.63 (d, 2H, J=8.5 Hz, ArH); MS (CI) m/z 468 (16), 450 (63), 296 (100); HRCIMS Calc'd for C₂₅H₂₅NO₆S: 468.1481; Found: 468.1467.

2-N-p-Toluenesulfonyl-4-(2-hydroxymethyl-3,4-dimethoxyphenyl)-1,2,3,4-tetrahydroisoquinoline (26). To a solution of compound 25 (1.4 g, 2.99 mmol) in dry THF (30 mL) was added 1.0 M borane-tetrahydrofuran (8 mL) at 0° C. under N₂. After the addition was complete the mixture was stirred at reflux overnight. Additional borane-tetrahydrofuran (4 mL) was added and stirring was continued for another 30 min. After cooling and evaporating under reduced pressure, methanol (30 “L) was carefully added, and the solvent was removed at low pressure. The process was repeated three times to ensure the methanolysis of the intermediate borane complex. Evaporation of the solvent gave 1.10 g (81%) of compound 26. An analytical sample was purified by flash chromatography (silica gel, EtOAc/Hexane) followed by recrystallization from ethyl acetate/hexane: mp 162-164° C.; ¹H NMR (CDCl₃) δ 2.38 (s, 3H, CH₃), 3.18 (dd, 1H, J=7.5, 11.9 Hz), 3.67 (dd, 1H, J=4.5, 11.8 Hz), 3.81 (s, 3H, OCH₃), 3.85 (s, 3H, OCH₃), 4.27 (d, 1H, J=15 Hz), 4.40 (d, 1H, J=15 Hz), 4.57 (t, 1H, J=6 Hz, CHAr₂), 4.71 (s, 2H, CH₂OH), 6.58 (d, 1H, J=8.5 Hz, ArH), 6.74 (d, 1H, J=8.6 Hz, ArH), 6.84 (d, 1H, J=7.7 Hz, ArH), 7.08 (t, 2H, J=7.6 Hz, ArH, 7.14 (t, 1H, J=6.6 Hz, ArH), 7.27 (d, 2H, J=8 Hz, ArH), 7.65 (d, 2H, J=8 Hz, ArH); MS (CI) m/z 454 (2.57), 436 (100).

8,9-Dimethoxy-2-p-toluenesulfonyl-2,3,7,11b-tetrahydro-1H-napth[1,2,3-de]isoquinoline (27). Powdered compound 26 (427 mg, 0.98 mmol) was added in several portions to 50 mL of cold concentrated sulfuric acid (50 mL) at −40° C. under nitrogen with vigorous mechanical stirring. A fter the addition, the reaction mixture was warmed to −5° C. over 2 hr and then poured onto crushed ice (450 g) and left stirring for 1 hr. The product was extracted with dichloromethane (2×150 mL), washed with water (2×150 mL), dried (MgSO₄), filtered, and evaporated to afford an oil that on trituration with ether at 0° C. yielded compound 27 (353 mg, 82%) as a white solid that was used without further purification. An analytical sample was prepared by centrifugal rotary chromatography using 50% EtOAc/hexane as the eluent followed by recrystallization from EtOAc/hexane: mp 204-206° C.; ¹H NMR (CDCl₃) δ 2.40 (s, 3H, CH₃), 2.80 (m, 1H, H-1a), 3.50 (dd, 1H, J=4.5, 17.5 Hz, H-1b), 3.70 (dd, 1H, J=7, 14 Hz, H-3a), 3.828 (s, 3H, OCH₃), 3.832 (s, 3H, OCH₃), 3.9 (m, 1H, H-11b), 4.31 (d, 1H, J=17.6 Hz, H-7a), 4.74 (ddd, 1H, J=1.7, 6.0, 11.2 Hz, H-7b), 4.76 (d, 1H, J=14.8 Hz, H-3b), 6.77 (d, 1H, J=8.3 Hz, ArH), 6.87 (d, 1H, J=8.4 Hz, ArH), 6.94 (d, 1H, J=7.6 Hz, ArH), 7.13 (t, 1H, J=7.5 Hz, Ar—H-5), 7.18 (d, 1H, J=7.2 Hz, ArH), 7.33 (d, 2H, J=8.1 Hz, ArH), 7.78 (d, 2H, J=8.2 Hz, ArH); MS (CI) m/z 436 (55), 198 (86), 157 (100); HRCIMS Calc'd for C₂₅H₂₅NO₄S: 436.1583; Found: 436.1570.

8,9-Dimethoxy-2,3,7,11b-tetrahydro-1H-napth[1,2,3-de]isoquinoline (28). A mixture of compound 27 (440 mg, 1.01 mmol), dry methanol (10 mL) and disodium hydrogen phosphate (574 mg, 4.04 mmol) was stirred under nitrogen at room temperature. To this mixture was added 6.20 g of 6% Na/Hg in three portions and the reaction was heated at reflux for 2 hr. After cooling, water (200 mL) was added and the mixture was extracted with ether (3×200 mL). The ether layers were combined, dried (MgSO₄), filtered (Celite), and evaporated to give an oil that solidified under vacuum. After rotary chromatography 142 mg (50%) of compound 28 was obtained as an oil. The oil quickly darkened on exposure to air and was used immediately. A small portion of the oil was treated with ethereal HCl and the hydrochloride salt of compound 28 was recrystallized from ethanol/ether: mp (HCl salt) 190° C. (dec.); ¹H NMR (CDCl₃, free base) δ 3.13 (dd, 1H, J=10.8, 12 Hz, H-1a), 3.50 (dd, 1H, J=3.4, 17.4 Hz, H-1b), 3.70 (m, 1H, H-11b), 3.839 (s, 3H, OCH₃), 3.842 (s, 3H, OCH₃), 4.03 (dd, 1H, J=6, 12 Hz, H-7a), 4.08 (s, 2H, H-3), 4.33 (d, 1H, J=17.4 Hz, H-7b), 6.78 (d, 1H, J=8.24 Hz, ArH), 6.92 (m, 3H, ArH), 7.11 (t, 1H, J=7.5 Hz, ArH), 7.18 (d, 1H, J=7.5 Hz, ArH); MS (CI) m/z 282 (100); BRCIMS Calc'd for C₁₈H₁₉NO₂: 282.1494; Found: 282.1497.

8,9-Dihydroxy-2,3,7,11b-tetrahydro-1H-napth[1,2,3-de]isoquinoline (29). To a solution of compound 28 (25 mg, 0.089 mmole) in dichloromethane (5 mL) at −78° C. was added boron tribromide (0.04 mL, 0.106 g, 0.42 mmol). After stirring at −78° C. under N₂ for 2 hr, the cooling bath was removed and the reaction mixture was left stirring at room temperature for 5 hr. It was then cooled to −78° C. and methanol (2 mL) was carefully added. After stirring for 15 min at room temperature, the solvent was evaporated. More methanol was added and the process was repeated three times. The resulting gray solid was recrystallized from ethanol/ethyl acetate to yield a total of 12 mg (41%) of the hydrobromide salt of compound 29: mp 258° C. (dec); ¹H NMR (HBr salt, CD₃OD) δ 3.43 (t, 1H, J=12 Hz, H-1a), 3.48 (dd, 1H, J=3.5, 18 Hz, H-1b), 4.04 (m, 1H, H-11b), 4.38 (dd, 2H, J=5.5, 12 Hz, H-7), 4.44 (s, 2H, H-3), 6.58 (d, 1H, J=8.5 Hz, ArH), 6.71 (d, 1H, J=8.5 Hz, ArH), 7.11 (d, 1H, J=7.5 Hz, ArH), 7.25 (t, 1H, J=7.5 Hz, ArH), 7.32 (d, 1H, J=7.5 Hz, ArH); MS (CI) m/z 254 (100); HRCIMS Calc'd for C₁₆H₁₅NO₂: 254.1181; Found: 254.1192.

### Example 11

### (R)-(+)-8,9-Dihydroxy-2,3,7,11b-tetrahydro-1H-napth[1,2,3-de]isoquinoline

5-Bromoisoquinoline. The apparatus was a 500 mL three-necked flask equipped with a condenser, dropping funnel, and a stirrer terminating in a stiff, crescent-shaped Teflon polytetrafluroethylene paddle. To the isoquinoline (57.6 g, 447 mmol) in the flask was added AiCl₃ (123 g, 920 mmol). The mixture was heated to 75-85° C. Bromine (48.0 g, 300 mmol) was added using a dropping funnel over a period of 4 hours. The resulting mixture was stirred for one hour at 75° C. The almost black mixture was poured into vigorously hand-stirred cracked ice. The cold mixture was treated with sodium hydroxide solution (10 N) to dissolve all the aluminum salts as sodium aluminate and the oily layer was extracted with ether. After being dried with Na₂SO₄ and concentrated, the ether extract was distilled at about 0.3 mm. A white solid (16.3 g, 78 mmol) from a fraction of about 125° C. was obtained (26% yield). The product was further purified by recrystallization (pentane or hexanes): mp 80-81° C.; ¹H NMR (DMSO-d₆) δ 9.34 (s, 1H), 8.63 (d, 1H, J=9.0 Hz), 8.17 (d, 1H, J=7,5 Hz), 8.11 (d, 1H, J=6.6 Hz), 7.90 (d, 1H, J=6.0 Hz), 7.60 (t, 1H, J=7.5 Hz); ¹³C NMR (DMSO-d₆) δ 153.0, 144.7, 134.3, 134.0, 129.3, 128.5, 128.0, 120.3, and 118.6. Anal. Calcd. for C₉H₆BrN: C, 51.96; H, 2.91; N, 6.73.

Found: C, 51.82; H, 2.91; N, 6.64.

5-Isoquinolinecarboxaldehyde. To a solution of n-butyllithium (19.3 mL of 2.5 M in hexanes, 48 mmol) in a mixture of ether (80 mL) and THF (80 mL) at −78° C. was added dropwise a solution of bromoisoquinoline (5.0 g, 24 mmol) in THF (10 mL). The reaction mixture was stirred at −78° C. under argon for 30 minutes. Following the general procedures described by Pearson, et al., in J. Heterocycl. Chem., Vol. 6 (2), pp. 243-245 (1969), a solution of DMF (3.30 g, 45 mmol) in THF (10 mL) was cooled to −78° C. and quickly added to the isoquinolyllithium solution. The mixture was stirred at −78° C. for 15 minutes. Ethanol (20 mL) was added followed by saturated NH₄Cl solution. The resulting suspension was warmed to room temperature. The organic layer, combined with the ether extraction layer, was dried over Na₂SO₄. A pale yellow solid (2.4 g, 15 mmol, 64% yield) was obtained from chromatography (SiO₂ Type-H, 50% EtOAc in hexanes) and recrystallization (ethanol): mp 114-116° C.; ¹H NMR (DMSO-d₆) δ 10.40 (s, 1H), 9.44 (s, 1H), 8.85 (d, 1H, J=6.0 Hz), 8.69(d, 1H, J=6.0 Hz), 8.45 (m, 2H), 7.90 (t, 1H, J=7.2 Hz); ¹³C NMR (DMSO-d₆) δ 194.23, 153.5, 146.2, 140.2, 135.2, 132.6, 130.2, 128.6, 127.5, and 117.2. Anal. Calcd. for C₁₀H₇NO.0.05H₂O: C, 75.99; H, 4.53; N, 8.86. Found: C, 75.98, H, 4.66; N, 8.68.

α-(5-Bromo-1,3-benzodioxol-4-yl)-5-isoquinolinemethanol. To a solution of 4-bromo-1,2-(methylendioxy)benzene (3.01 g, 15 mmol) in THF (20 mL) at −78° C. was added dropwise lithium diisopropylamide (10.6 mL of 1.5 M in cyclohexane, 16 mmol). The reaction mixture was stirred at −78° C. under argon for 20 minutes. A brown solution was formed. A solution of 5-isoquinolinecarboxaldehyde (1.90 g, 12 mmol) in THF (4 mL) was added dropwise. The resulting mixture was stirred at −78° C. for 10 minutes and warmed to room temperature. Stirring was continued for 30 minutes at room temperature, and then the mixture was quenched with saturated NH₄Cl solution. The product was extracted with EtOAc and the solvent was removed under reduced pressure. Chromatography (SiO₂ Type-H, 35% EtOAc in Hexanes) of the residue yielded the title compound as a yellow solid (2.8 g, 7.8 mmol, 65% yield): mp 173-175° C.; ¹H NMR (DMSO-d₆) δ 9.32 (s, 1H), 8.47 (d, 1H, J=6.0 Hz), 8.05 (d, 1H, J=8.1 Hz), 7.96 (d, 1H, J=7.2 Hz), 7.76 (d, 1H, I=6.0 Hz), 7.66 (t, 1H, J=7.8 Hz), 7.14 (d, 1H, =8.1 Hz), 6.84 (d, 1H, J=8.1 Hz), 6.58 (d, 1H, J=8.1 Hz), 6.28 (d, 1H, J=5.4 Hz), 5.95 (s, 1H), 5.80 (s, 1H); ¹³C NMR (DMSO-d₆) δ 153.1, 147.6, 147.0, 142.9, 136.9, 132.7, 128.9, 128.3, 127.3, 126.7, 125.6, 124.4, 116.3, 114.0, 109.3, 101.6, and 69.0. Anal. Calcd. for C₁₇H₁₂BrNO₃: C, 57.01; H, 3.38; N, 3.91. Found: C, 57.04; H, 3.51; N, 3.89.

5-[(5-Bromo-1,3-benzodioxol-4-yl)methyl]isoquinoline. To a solution of secondary alcohol a-(5-bromo-1,3-benzodioxol-4-yl)-5-isoquinolinemethanol (8.37 mmol) in trifluoroacetic acid (100 mL), triethylsilane (83.7 mmol) was added and the resulting solution was refluxed for an hour at 70-75° C. and stirred overnight at room temperature. The solvent was removed under vacuum and the residue was dissolved in ethyl acetate, washed with saturated NH₄Cl dried over Na₂SO₄, filtered, and concentrated. Purification was performed by column chromatography to afford the trifluoroacetate salt of the title compound as a white crystalline solid (67% yield): mp 138-140° C.; ¹H NMR (CDCl₃) δ 9.64 (s, 1H), 8.63 (d, 1H, J=6.59 Hz), 8.45 (d, 1H, J=6.62 Hz), 8.14 (d, 1H, J=8.22 Hz), 7.77 (t, 1H, J=7.39 HZ), 7.64 (d, 1H, J=7.29 Hz), 7.13 (d, 1H, J=8.33 Hz), 6.71 (d, 1H, J=8.31 Hz), 5.94 (s, 2H), 4.53 (s, 2H); ¹³C NMR (CDCl₃) δ 147.8, 147.7, 147.1, 137.2, 135.1, 134.7, 133.4, 130.3, 128.6, 128.3, 125.9, 120.7, 119.4, 116.3, 109.1, 101.2 and 31.7. Anal. Calcd. for C₁₇H₁₂BrNO₂.C₂HF₃O₂: C, 50.02; H, 2.87; Br, 17.51; N. 3.07. Found: C, 49.91; H, 3.02; Br, 17.95; N, 3.04.

Method A for 12H-Benzo[d,e][1,3]benzodioxol[4,5-h]isoquinoline. A solution of 5-[(5-bromo-1,3-benzodioxol-4-yl)methyl]-isoquinoline (0.357 g, 1.0 mmol) and 2,2′-azobisisobutylronitrile (0.064 g, 0.39 mmol) in benzene (10 mL) was cooled to −78° C., degassed four times with N₂ and then heated to 80° C. under argon. A solution of tributyltin hydride (1.14 g, 3.9 mmol) in 10 mL of degassed benzene was added in two hours. TFA (0.185 g, 1.6 mmol) was added in four equal portions (¼ each half hour). The reaction mixture was stirred at 80° C. under argon for six hours after addition of TFA. Additional tributyltin hydride (0.228 g, 0.80 mmol) was added dropwise. The stirring continued overnight (16 hours). Another 2,2′-azobisisobutylronitrile (0.064 g, 0.39 mmol) and TFA (0.093 g, 0.80 mmol) were added in one portion. A solution of tributyltin hydride (1.14 g, 3.9 mmol) in 10 mL of degassed benzene was also added in two hours. More TFA (0.185 g, 1.6 mmol) was added in four equal portions (1/4 each half hour). The stirring continued for another six hours and tributyltin hydride (0.456 g, 1.6 mmol) was added dropwise. The reaction mixture was stirred overnight (16 hours). The solvent was removed under reduced pressure. Pentane (100 mL) was added to the residue and the resulting mixture was cooled to −78° C. A brown gum was formed and filtered. The filtrate was extracted with MeCN. The MeCN layer was combined with the brown gum. The crude product from evaporation of MeCN was purified by chromatography (SiO₂ Type-H, 15% EtOAc in hexanes). The isolated compound was dissolved in CH₂Cl₂ and extracted with HCl (1N). The aqueous layer was basified to pH˜10 using 10 N NaOH solution and reextracted with CH₂Cl₂. The organic layer was dried over Na₂SO₄. Evaporation of solvent yielded the title compound as an orange solid (0.068 g, 0.26 mmol, 25% yield): mp 194-197° C.; ¹H NMR (DMSO-d₆) δ 9.12 (s, 1H), 9.06 (s, 1H), 7.93 (d, 1H, J=6.9 Hz), 7.83 (d, 1H, J=8.1 Hz), 7.73 (dd, 1H, J=7.2, 1.5 Hz), 7.66 (t, 1H, J=7.8 Hz), 6.96 (d, 1H, J=8.4 Hz), 6.14 (s, 2H), 4.44 (s, 2H); ¹³C NMR (DMSO-d₆) δ 150.6, 147.0, 145.2, 135.6, 130.6, 129.3, 129.1, 127.7, 127.5, 125.0, 123.6, 117.2, 116.1, 107.5, 101.6, and 26.6. Anal. Calcd. for Cl₇H₁₁NO₂.0.12CH₂Cl₂: C, 75.75; H, 4.17; N, 5.16. Found: C, 75.75; H, 4.03; N, 4.83.

Method B. A solution of 5-[(5-bromo-1,3-benzodioxol-4-yl)methyl]-isoquinoline (12.6 g, 36.8 mmol) and 2,2′-azobisisobutylronitrile (5.92 g, 36.0 mmol) in benzene (1500 mL) was cooled to −78° C., degassed/purged four times with nitrogen and then heated to 80° C. under argon. A solution of tributyltin hydride (39.9 g, 137 mmol) in 30 mL of degassed benzene was added dropwise over a period of three hours. Acetic acid (12.5 g, 210 mmol) was added in one portion before the addition of tin hydride. The reaction mixture was stirred at 80° C. under argon for 16 hours. Excess triethylamine was added to neutralize the residual acetic acid component. The solvent was removed under reduced pressure. Methylene chloride (250 mL) was added to dissolve the semi-solid, followed by the addition of hexanes to a point just before the mixture became cloudy. This solution was poured over a short bed of silica gel and the tri-n-butyltin acetate was removed by washing with hexanes until no longer detected by TLC. The product was then eluted out with mixtures of hexanes and ethyl acetate to give the desired title compound (6.1 g, 23.4 mmol, 63.5% yield) which was identical to the product prepared by Method A.

Method A for (±)-8,9-Methylenedioxy-2,3,7,11b-tetrahydro-1H-napth[1,2,3-de]isoquinoline. To a solution of 12H-benzo[d,e][1,3]benzodioxol[4,5-h]isoquinoline (0.085 g, 0.33 mmol) in THF (43 mL) was added 2N HCl (1.7 mL, 3.4 mmol) and an orange precipitate formed. Sodium cyanoborohydride (0.274 g, 44 mmol) was added in one portion. The resulting suspension was stirred at room temperature for two hours. HCl (2N, 10 mL) was added and stirring continued for 5 minutes. Saturated NaHCO₃ solution was added (pH˜7-8). The resulting mixture was extracted with EtOAc, dried over Na₂SO₄ and the solvent was removed under reduced pressure. Chromatography (SiO₂ Type-H, 5% MeOH in CH₂Cl₂) of the residue yielded the title compound as a yellow gum (0.066 g, 0.25 mmol, 75% yield);

¹H NMR (CDCl₃) δ 7.15 (m, 2H), 6.97 (d, 1H, J=6.9 Hz), 6.83 (br, s, 1H), 6.68 (d, 1H, J=8.1 Hz), 6.59 (d, 1H, J=8.1 Hz), 6.01 (d, 1H, J=1.4 Hz), 5.91 (d, 1H, J=1.4 Hz), 4.40-4.00 (m, 5H), 3.55 (dd, 1H, J=17.7, 3.0 Hz), 3.10 (t, 1H, J=12.0 Hz); ¹³C NMR (CDCl₃) δ 146.1, 144.8, 136.0, 132.2, 130.4, 128.6, 127.1, 127.0, 124.5, 118.5, 116.2, 106.2, 101.2, 45.8, 35.1, 34.3, and 28.9. Anal. Calcd. for C₁₇H₁₅NO₂e0.52HCN.1.8H₂O: C, 67.49; H, 6.18; N, 6.83. Found: C, 67.45; H, 5.96; N, 6.75.

Method B. 12H-Benzo[d,e][1,3]benzodioxol[4,5-h]isoquinoline (11.26 g) was dissolved into 500 mL of glacial acetic acid in a suitable glass liner that will fit into a 1-L Parr “bomb reactor.” To this dark amber solution was added 480 mg PtO2 and a magnetic stirring bar. Usual purge cycles were repeated three times at −78° C. Finally hydrogen gas was charged into the steel bomb at 140 PSI while the content was still at −78° C. The reactor was allowed to warm to room temperature over a period of 2 hours while the internal pressure increased to 195 PSI. Gas absorption was faster after about 4 hours at room temperature. After 24 hours, the internal pressure returned to 165 PSI indicating roughly stoichiometric uptake of hydrogen gas. The black suspension was removed after the pressure was relieved, filtered over silica gel, rinsed with acetic acid, and concentrated under reduced pressure to give about 19 gm of gummy substance. The crude product was neutralized with sodium bicarbonate solution followed by extraction with methylene chloride to yield 11.6 gm of the title compound whose ¹H NMR was indistinguishable from the purified material prepared above by the Method A.

(±)-8,9-Dihydroxy-2,3,7,11b-tetrahydro-1H-napth[1,2,3-de]isoquinoline. BBr₃ (25.0 mL of 1 M in CH₂Cl₂, 25.0 mmol) was added to a cooled solution (−78° C.) of methylenedioxy dinapsoline as prepared in Example 6 (1.4 g, 5.3 mmol) in CH₂Cl₂. The mixture was stirred at −78° C. under nitrogen for three hours and then at room temperature overnight. After the mixture was cooled to −78° C., methanol (50 mL) was added dropwise and the solvent was removed by reduced pressure. The residue was dissolved in methanol (100 mL) and the solution was refluxed under nitrogen for 2 hours. After removal of solvent, chromatography (SiO₂, 10% MeOH in CH₂Cl₂) of the residue yielded the title compound as a dark brown solid (1.65 g, 4.94 mmol, 93% yield). MS (ESI) m/z 254 (MH⁺); ¹H NMR (DMSO-d₆) δ 9.50 (br, s, 2H), 9.28 (s, 1H), 8.54 (s, 1H), 7.32 (d, 1H, J=8.3 Hz); 7.23 (t, 1H, J=8.3 Hz), 7.12 (d, 1H, J=8.5 Hz), 6.70 (d, 1H, J=9.3 Hz), 6.54 (d, 1H, J=6.7 Hz), 4.37 (s 2H), 4.30-4.23 (m, 2H), 3.97 (m, 1H), 3.45-3.31 (m, 2H);”³C NMR (DMSO-d₆) δ 143.8, 142.0, 136.9, 132.1, 127.6, 127.0, 126.6, 124.1, 123.7, 114.0, 112.7, 46.6, 44.0, 32.9, and 28.5. Anal. Calcd. for C₁₆H₁₅NO₂.1.28HBr.0.59H₂O: C, 52.34; H, 4.79; N, 3.82. Found: C, 52.29; H, 4.92; N, 4:14.

### R-(+)-8,9-Dihydroxy-2,3,7,11b-tetrahydro-1H-napth[1,2,3-de]isoquinoline

Step A. (+)-8,9-Methylenedioxy-2,3,7,11b-tetrahydro-1H-napth[1,2,3-de]isoquinoline. A sample of racemic (+)-8,9-methylenedioxy-2,3,7,11b-tetrahydro-1H-napth[1,2,3-de]isoquinoline was injected into a preparative HPLC (Dynamax Rainin Model SD-1) equipped with Chiralcel OD column (5 cm×50 cm, 20μ, Chiral Technologies, Inc) at a flow rate of 50 mL/min using UV detector set at λ=220 nm. Using an isocratic method, the solvent system (5% Ethanol/Hexanes, 0.1% TFA) was found to best separate the enantiomers. As much as 150 mg/5 mL ethanol can be injected to the column per run. A total of 425 mg of racemic (+)-8,9-methylenedioxy-2,3,7,11b-tetrahydro-1H-napth[1,2,3-de]isoquinoline injected can produce about 200 mg of each enantiomer. Optical rotation was taken for each of the enantiomer collected: 1^{st} Peak (Rf=19.6 minutes): [α]_{D} −88.9° (c 0.03, CHCl₃); 2^{nd} Peak (Rf=23.6 minutes): [α]_{D} −90.3° (c 0.03, CHCl₃).

One of these two isomers was derivatized into the corresponding N-(p-tolylsulfonamide) for a single crystal X-ray determination. From there it was concluded that the chirality of the (−)-isomer of Formula VIIb has (S)-configuration at the asymmetric center. The second peak is the desired title compound.

Step B. R-(+)-8,9-Dihydroxy-2,3,7,11b-tetrahydro-1H-napth[1,2,3-de]isoquinoline

Using the identical deprotection procedure described for the racemic compound in Example 7, each of these isomers were subjected to BBr₃ deprotection to give chiral (+) and (−)-isomers of dinapsolines (DNS).

| | DNS from first peak | DNS from second peak |
|---|---|---|
| Optical | −70.7° (c 0.03, MeOH) | +75.0° (c 0.03, MeOH) |
| rotations [α]_{D} | | |

### (R)-(+)-8,9-Dihydroxy-2,3,7,11b-tetrahydro-1H-napth[1,2,3-de]isoquinoline

Step A. (±)-8,9-Methylenedioxy-2,3,7,11b-tetrahydro-1H-napth[1,2,3-de]isoquinoline A solution of racemic (O)-8,9-methylenedioxy-2,3,7,11b-tetrahydro-1H-napth[1,2,3-de]isoquinoline (3.0 gm, 11.3 mmol) in 100 mL of 95% ethyl alcohol at room temperature was mixed with a warm solution of (+)-dibenzoyl-D-tartaric acid in 40 mL of 95% ethyl alcohol. The solution was allowed to stand at room temperature for 4 hours and the grayish off-white crystals were collected by filtration and subsequently dried in a vacuum oven at 35° C. to give 1.3 gm (melting point: 175-176° C., 35.7%). The enantiomeric purity was determined by the same chiral HPLC conditions described above in Example 8: the salt was neutralized with 2M potassium hydroxide solution and the organic materials extracted with methylene chloride. The organic layers were combined and concentrated under reduced pressure to give a white solid which was redissolved in methanol prior to injection into HPLC Chiral column. The ratio of the second peak to the first was determined to be greater than 40:1. The identical resolution may also be carried out using the unnatural D-tartaric acid. Melting points are uncorrected for the desired tartaric salts of the title compound. (R)-(+)-(+)-8,9-methylenedioxy-2,3,7,11b-tetrahydro-1H-napth[1,2,3-de]isoquinoline(+)-dibenzoyl-D-tartaricacidsalt: mp 175-176° C. (R)-(+)-(+)-8,9-methylenedioxy-2,3,7,1 b-tetrahydro-1H-napth[1,2,3-de]isoquinoline D-tartaric acid salt: mp 186-188° C.; [α]²⁵=+90.3°.

Step B. (R)-(+)-8,9-dihydroxy-2,3,7,11b-tetrahydro-1H-napth[1,2,3-de]isoquinoline

The free base is regenerated from the tartaric salts by neutralization. The (+)-isomer of dinapsoline prepared by deprotection as described in Example 7 is identical to the (+)-isomer of Example 8.

### Formulation Example 1

### Hard gelatin capsules

| | mg/capsule |
|---|---|
| Compound 6a | 10 mg |
| Olanzapine | 25 |
| Starch, dried | 150 |
| Magnesium stearate | 10 |
| Total | 210 |

### Formulation Example 2

### Tablets

| | mg/tablet |
|---|---|
| Compound 6b | 10 mg |
| Olanzapine | 10 |
| Cellulose, microcrystalline | 275 |
| Silicon dioxide, fumed | 10 |
| Stearic acid | 5 |
| Total | 310 |

The components are blended and compressed to form tablets each weighing 465 mg.

### Formulation Example 3

### Aerosol solution

| | |
|---|---|
| Compound 6c | 1 mg |
| Risperidone | 5 mg |
| Ethanol | 25.75 mg |
| Propellant 22 ((Chlorodifluoromethane)) | 60.00 mg |
| Total | 100.75 mg |

The active compound is mixed with ethanol and the mixture added to a portion of the propellant 22, cooled to −30° C. and transferred to a filling device. The required amount is then fed to a stainless steel container and diluted with the remainder of the propellant. The valve units are then fitted to the container.

### Formulation Example 4

### Tablets

| Compound 6d | 10 | mg |
|---|---|---|
| Sertindole | 60 | mg |
| Starch | 30 | mg |
| Microcrystalline cellulose | 20 | mg |
| Polyvinylpyrrolidone | 4 | mg |
| | (as 10% solution in water) | |
| Sodium carboxymethyl starch | 4.5 | mg |
| Magnesium stearate | 0.5 | mg |
| Talc | 1 | mg |
| Total | 140 | mg |

The active ingredient, starch and cellulose are passed through a No. 45 mesh U.S. sieve and mixed thoroughly. The aqueous solution containing polyvinyl-pyrrolidone is mixed with the resultant powder, and the mixture then is passed through a No. 14 mesh U.S. sieve. The granules so produced are dried at 50° C. and passed through a No. 18 mesh U.S. sieve. The sodium carboxymethyl starch, magnesium stearate and talc, previously passed through a No. 60 mesh U.S. sieve, are then added to the granules which, after mixing, are compressed on a tablet machine to yield tablets each weighing 170 mg.

### Formulation Example 5

### Capsules

| | |
|---|---|
| Compound 6e | 10 mg |
| Quetiapine | 70 mg |
| Starch | 39 mg |
| Microcrystalline cellulose | 39 mg |
| Magnesium stearate | 2 mg |
| Total | 140 mg |

The active ingredient, cellulose, starch, and magnesium stearate are blended, passed through a No. 45 mesh U.S. sieve, and filled into hard gelatin capsules in 250 mg quantities.

### Formulation Example 6

### Suppositories

| | |
|---|---|
| Compound 16a | 10 mg |
| Ziprasidone | 75 mg |
| Saturated fatty acid glycerides | 2,000 mg |
| Total | 2,080 mg |

The active ingredient is passed through a No. 60 mesh U.S. sieve and suspended in the saturated fatty acid glycerides previously melted using the minimum heat necessary. The mixture is then poured into a suppository mold of nominal 2 g capacity and allowed to cool.

### Formulation Example 7

### Suspensions

| | | |
|---|---|---|
| Compound 16b | 10 | mg |
| Olanzapine | 20 | mg |
| Sertraline | 100 | mg |
| Sodium carboxymethyl cellulose | 50 | mg |
| Syrup | 1.25 | ml |
| Benzoic acid solution | 0.10 | ml |

| | |
|---|---|
| Flavor | q.v. |
| Color | q.v. |
| Purified water | to total 5 ml |

The active ingredient is passed through a No. 45 mesh U.S. sieve and mixed with the sodium carboxymethyl cellulose and syrup to form a smooth paste. The benzoic acid solution, flavor and color are diluted with a portion of the water and added, with stirring. Sufficient water is then added to produce the required volume.

### Formulation Example 8

### Intravenous Formulation

| | | |
|---|---|---|
| Compound 16c | 1 | mg |
| Olanzapine | 20 | mg |
| Isotonic saline | 1000 | ml |

### Method Example 1

The affinity of the compounds described in Examples 1, 2, 3, and 5 for D₁ and D₂ receptors was assayed utilizing rat brain striatal homogenates having D₁ and D₂ receptors labeled with ³H—SCH 23390 and ³H-spiperone, respectively. The data obtained are shown in Table 1.

TABLE 1

| | D₁ | D₂ | D₁:D₂ |
|---|---|---|---|
| Compound | Affinity ^{(a)} | Affinity ^{(a)} | Selectivity |
| 6a | 8 | 100 | 13 |
| 6b | 14 | 650 | 46 |
| 6c | 7 | 45 | 6 |
| 6e | 290 | 185 | 0.6 |

(a) Affinity in nM.

### Method Example 2

### Passive Avoidance Assay

### Passive Avoidance in Rats

The protocol summarized below is one of many variants of the passive avoidance procedure using scopolamine-induced amnesia (for review see Rush, Behav Neural Biol 50:255-274, 1988). This procedure is commonly used to identify drugs that may be useful in treating cognitive deficits, particularly those observed in AD. The effects of the D.sub. 1 agonist DHX in this assay were evaluated to demonstrate the potential of this class of drugs to treat dementia.

Testing was conducted in standard 2-compartment rectangular passive avoidance chambers (San Diego Instruments, San Diego, Calif.) with black plexiglas sides and grid floors. The light compartment of the chambers were illuminated by a 20 W lamp located in this compartment; the dark side of the chambers will be shielded from light, except for light penetrating the opening connecting the two compartments of each chamber.

On training day, groups of 8 rats were injected with scopolamine (3.0 mg/kg, ip) or vehicle (1.0 ml/kg) 30 min prior to training. Scopolamine served as the dementing agent in this experiment. Ten min prior to training, each group of rats received a second injection of vehicle or a dose of DHX. At the end of the pretreatment interval, each rat was placed individually in the light compartment facing away from the opening between compartments. The latency for each rat to travel from the light to the dark compartment was measured up to a maximum of 300 sec; any animal not entering the dark compartment within 300 sec was discarded from the test group. Once the animal entered the dark compartment completely, a 1.0 milliampere, 3.0 sec scrambled shock was delivered to the entire grid floor. The animal was allowed to remain in the dark compartment during this 3.0 sec period or to escape to the light compartment. Each rat was then returned immediately to its home cage.

Twenty-four hr after training, each rat was tested in the same apparatus for retention of the task (to remain passively in the light compartment). The procedure on test day was identical to that of the training day, except that no injections were given and that the rats did not receive a shock upon entering the dark compartment. The latency for animals to enter the dark compartment on test day (step-through latency) was recorded up to a maximum of 600 sec. Each animal was used only once in a single experiment.

A one-way analysis of variance (ANOVA) and Newman-Keuls post-hoc comparisons were used to identify significant deficits in passive avoidance responding produced by scopolamine and their reversal by DHX; a p value of less than 0.05 was used as the level of significance.

Scopolamine (3.0 mg/kg) produced a severe deficit in the acquisition of the passive avoidance task. DHX significantly improved scopolamine-induced deficits in step-through latency at a dose of 0.3 mg/kg (FIG. 1). Doses of 0.1 and 1.0 mg/kg of DHX also increased step-through latency, however, these increases were not statistically-significant. These results are similar to those obtained with drugs such as physostigmine which have been used in the treatment of AD. These results are also consistent with the hypothesis that dopamine D.sub. 1 agonists may be effective in the treatment of dementia.

Dihydrexidine significantly improved the deficits induced by scopolamine over a narrow range of doses (0.1, 0.3, and 1.0 mg/kg ip). Dihydrexidine produced an inverted U-shaped dose-response curve, typical of potential cognitionenhancing agents in this procedure. The improvement in cognitive performance may be due to D₁ dopamine receptor-mediated increases in acetylcholine release induced by dihydrexidine in brain regions involved in cognition (e.g., frontal cortex). Dihydrexidine has been found to produce dose-related increases in acetylcholine release in the striatum and frontal cortex of conscious, freely-moving rats using in vivo microdialysis.

### Method Example 3

### MPTP-Treated Monkeys as a Model for Parkinson's Disease Subjects and Behavioral Testing

Two adult male Macaca fascicularis monkeys (4.7 and 5.7 kg initial body weight) and 1 female Macaca nemistrina monkey (5.0 kg initial body weight) were trained to perform a delayed response task. Briefly, animals were trained and tested on delayed response while seated in a restraining chair placed inside a sound attenuating modified Wisconsin General Test Apparatus. The monkey sat behind an opaque screen that when raised, allowed access to a sliding tray that contained recessed food wells with identical sliding white Plexiglas covers that served as stimulus plaques that could be displaced by the animal to obtain rewards (e.g. raisins). Monkeys were trained to retrieve a raisin from one of the food wells after observing the experimenter bait the well. Right and left wells were baited in a randomized, balanced order. Animals were maintained on a restricted diet during the week and tested while food deprived.

Training was accomplished with a non-correction procedure, beginning with a 0 s delay and progressing to a 5 s delay. Animals were trained until performance with a 5 s delay was 90% correct or better for at least 5 consecutive days. Each daily session consisted of 25 trials. A response was scored a “mistake” if the monkey made its response choice to a well that was not baited with reward. A “no response” error was scored if the monkey failed to respond to a trial within 30 s.

### Toxin Administration

Once animals were performing at criterion level, MPTP administration began. MPTP-HCl (in sterile saline) was administered intravenously two or three times per week while animals were seated in the restraining chair with an ankle cuff limiting movement of one leg. The monkeys were trained to allow the experimenter to hold one leg and to not struggle during intravenous injection into the saphenous vein. Personnel administering MPTP wore a disposable gown, latex gloves, and a face mask with a splash shield. Following administration of the toxin, the used syringe was filled with a saturated solution of potassium permanganate (to oxidize any remaining MPTP), capped, and discarded as hazardous waste. Waste pans located beneath the animal's cages and any excreta located in those pans were sprayed with a potassium permanganate solution prior to disposal of the excreta. Laboratory animal care personnel took care not to generate aerosols during cage cleaning.

MPTP was administered to each animal in doses ranging from 0.05 mg/kg at the start of the study to 0.20 mg/kg. Animals received cumulative MPTP doses of 64.7 mg, 23.9 mg, and 61.7 mg on a variable dosing schedule over periods of 346 days, 188 days, and 341 days, respectively. The different total amounts of MPTP administered reflect variability in individual animal sensitivity and response to the toxin. Although animals received different total amounts of toxin over different time periods, the nature of the cognitive deficits were similar in all animals.

### Drug Administration

Pharmacological data were obtained after animals consistently showed at least a 15% performance deficit on delayed response. Compounds and/or compositions described herein are tested by dissolving in physiological saline containing 0.2% ascorbate and administering subcutaneously. Ilustratively, compounds and/or compositions described herein are used at 0.3, 0.6, and 0.9 mg/kg doses, calculated as the free base where appropriate. The order of dose administration is determined randomly. Each dose is tested at least twice in each animal. On some trials, compounds and/or compositions described herein are administered in combination with the dopamine D-1 receptor antagonist, such as SCH-23390 (0.0075 or 0.015 mg/kg). On such trials, SCH-23390 is administered 15 min prior to the compounds and/or compositions described herein.

Delayed response testing begins 8 min after compounds and/or compositions administration. On drug testing days, animals are tested for delayed response performance, administered compounds and/or compositions (or saline), and re-tested on the delayed response task. Saline control trials are performed approximately once every third test session. Saline injections control for effects of receiving an injection and for possible changes in performance as a consequence of being tested a second time in one day. A minimum of 3 days separate compounds and/or compositions trials in any particular animal. Compounds and/or compositions test sessions are conducted only if subjects meet the 15% or more performance deficit requirement on any particular day.

### Data Analysis

Delayed response performance after dihydrexidine administration was compared with matched control performance obtained on the same day prior to drug administration. The total number of correct responses as well as the number of mistakes and “no response” errors were tabulated for each test session. Data were then expressed as mean (_+standard deviation) performance. All animals served as their own controls and statistical analyses consisted of analysis of variance, repeated measures design, with post hoc comparisons (Bonferroni t test).

### Method Example 4

### OHDA or Reserpine Treated Monkeys

This assay is used to assess cognitive function, and is gemerally described in Amsten et al., Psychopharmacol. 116:143-51 (1994); the disclosure of that assay is incorporated herein by reference.

### Method Example 5

### C-6 glioma cells transfected with the rhesus macaque D1A receptor (C-6-mD_{1A})

Cells were grown in DMEM-H medium containing 4,500 mg/l glucose, L-glutamine, 5% fetal bovine serum and 600 ng/ml G418. In the present studies, the density of mD_{1A} receptor binding sites in untreated cells was approximately 50 fmol/mg protein for C-6-mDlA cells. Cells were plated into 24-well plates and allowed to grow to confluence (usually 2-4 days), after which they were used for either dose-response or desensitization studies. For the binding studies, 75-cm² flasks of confluent cells were treated as described below. All studies (functional and receptor binding) used cells from passages 2 to 20. Cells were maintained in a humidified incubator at 37° C. with 95% O₂ and 5% CO₂.

### Method Example 6

### Dose-Response Studies

Agonist intrinsic activity was assessed by the ability of selected compounds to stimulate adenylate cyclase, as measured by cAMP accumulation in whole cells. Confluent plates of cells were incubated with drugs dissolved in DMEM-H supplemented with 20 mM HEPES, 0.1% ascorbic acid and 500 μM IBMX (pH 7.2; media A). The final volume for each well was 500 μl. In addition to the dose-response curves run for each drug, basal levels of cAMP and isoproterenol-stimulated cAMP accumulation were evaluated for each plate. Each condition was run in duplicate wells. After a 10-min incubation at 37° C., cells were rinsed briefly with media, and the reaction was stopped by the addition of 500 μl of 0.1 N HCl. Cells were then allowed to chill for 5 to 10 min at 4° C., the wells were scraped, and the contents placed into 1.7-ml centrifuge tubes. An additional 1 ml of 0.1 N HCl was added to each tube, for a final volume of 1.5 ml/tube. Tubes were vortexed briefly, and then spun in a BHG HermiLe Z 230 M microcentrifuge for 5 min at 15,000×g to eliminate large cellular particles. Cyclic AMP levels for each sample were determined radioimmunoassay.

### Method Example 7

### Receptor Desensitization Assay

Plates of confluent cells were incubated with test drugs dissolved in plain DMEM-H media supplemented with 20 mM HEPES and 0.1% ascorbic acid (pH 7.2; media B). Cells, in a final volume of 500 μl/well, remained in the incubator during the desensitization period. At the end of the desensitization period, cells were rinsed for 30 min at 37° C. with 500 μl of media B. Cells were then challenged with 10 μM dopamine (dissolved in media A) for 10 min at 37° C., followed by a brief rinse with 500 μl of media A. The reaction was stopped with the addition of 500 μl of 0.1 N HCl, the plates were scraped and the contents placed into 1.7-ml centrifuge tubes. After vortexing briefly, these tubes were centrifuged and then cyclic AMP levels were evaluated by RIA. Basal activity (i.e., in the absence of drug) was measured before and after incubation with each concentration of test drug.

### Method Example 8

### Radioimmunoassay of cAMP

The concentration of cAMP in each sample was determined with an RIA of acetylated cAMP (modified as described by Harper & Brooker, J. Cyclic Nucleotide Res. 1:207-218 (1975). Iodination of cAMP was performed according to Patel and Linden, Anal. Biochem. 168:417-420 (1988). Assay buffer was 50 mM sodium acetate buffer with 0.1% sodium azide (pH 4.75). Standard curves of cAMP were prepared in buffer at concentrations of 2 to 500 fmol/assay tube. To improve assay sensitivity, all samples and standards were acetylated with 10 μl of a 2:1 solution of triethylamine/acetic anhydride. Samples were assayed in duplicate. Each assay tube contained 10 μl of sample, 100 μl of buffer, 100 μl of primary antibody (sheep, anti-cAMP, 1:100,000 dilution with 1% BSA in buffer) and 100 μl of [¹²⁵I]cAMP (50,000 dpm/100 μl of buffer); total assay volume was ˜300 μl. Tubes were vortexed and stored at 4° C. overnight (approximately 18 hr). Antibody-bound radioactivity then was separated by the addition of 10 μl of BioMag rabbit, anti-goat IgG (Advanced Magnetics, Cambridge Mass.), followed by vortexing and further incubation at 4° C. for 1 hr. To these samples 1 ml of 12% polyethylene glycol/50 mM sodium acetate buffer (pH 6.75) was added, and all tubes were centrifuged at 1700×g for 10 min. Supernatants were aspirated and radioactivity in the resulting pellet was determined with an LKB Wallac gamma counter (Gaithersburg, Md.).

### Method Example 9

### Analysis of Affinity for Agonists at C-6-mDIA Receptors

Flasks of cells in the same passage were rinsed with 5 ml hypoosmotic buffer (1 mM HEPES, 2 mM EGTA, pH 7.4), and then incubated with 7 ml hypoosmotic buffer for 5 to 10 min at 4° C. Cells were then scraped off the bottom of the flask with a rubber policeman, collected into 50-ml tubes and centrifuged at 28,000×g at 4° C. for 20 min. The resulting pellet was resuspended in binding buffer (50 mM IEPES, pH 8.0), homogenized with a Brinkmann Polytron on a setting of 5 for 10 sec, and either used immediately or stored in 1-ml aliquots at −80° C. until use in binding assays. Aliquots contained approximately 1 mg/ml of protein, as measured with the BCA protein assay reagent (Pierce, Rockford, Ill.).

Competition binding studies were done to evaluate the affinity of the different agonists for the mD1A receptor. Membranes were diluted in assay buffer A (50 mM HEPES, 0.9% NaCl, pH 8.0) and 100 μl of membranes (approximately 50 μg) was incubated with 0.3 nM [3H]SCH23390 (prepared according to Wyrick et al., J Labelled Compd. Radiopharm. 23:685-692 (1986), specific activity, 85 Ci/mmol, the disclosure of which is incorporated herein by reference) and increasing concentrations of competing drug (0.01 nM-1 μM) in assay buffer B (50 mM HEPES, 0.9% NaCl, 0.001% BSA, pH 8.0). BSA was omitted from assay buffer A to determine protein levels in the samples accurately. (BSA was used as the standard in protein determinations.) Nonspecific binding was determined by 5 μM SCH23390, because there is no binding of SCH23390 in wild-type cells. Tubes were run in triplicate in a final volume of 500 μl. After incubation at 37° C. for 15 min, tubes were filtered rapidly through Skatron glass fiber filter mats (11734) and rinsed with 5 ml of ice-cold wash buffer (10 mM Tris, 0.9% NaCl, pH 7.4) with a Skatron Micro Cell Harvester (Skatron Instruments Inc., Sterling, Va.). Filters were allowed to dry, then punched into scintillation vials (Skatron Instruments Inc., Sterling, Va.). OptiPhase ‘HiSafe’ II scintillation cocktail (1 ml) was added to each vial. After shaking for 30 min, radioactivity in each sample was determined on an LKB Wallac 1219 Rackbeta liquid scintillation counter.

### Method Example 10

### Effect of Agonist Exposure on D₁ Receptor Expression Levels

Flasks of cells in the same passage were exposed to 7 ml media B, or 7 ml media B supplemented with 10 μM concentrations of the various drugs for 2 hr. Cells were then rinsed with 7 ml media B (30 min), and then membranes were prepared as described above. Saturation binding studies were done to evaluate the level of expression of receptors in control and desensitized membranes and were the same as the competition studies with the following modifications. Membranes were diluted in assay buffer A and 100 μl of membranes (approximately 50 μg) was incubated with six concentrations of [³H]SCH23390 (0.09-1.1 nM), prepared in assay buffer B. Nonspecific binding was determined using 5 μM SCH23390.

### Method Example 11

### Data Analysis

For dose-response studies, data were calculated for each sample and expressed initially as pmol cAMP per mg protein per min. Base-line values of cAMP were subtracted from the total amount of cAMP produced for each drug condition. To minimize inter-assay variation, data for each drug were expressed relative to the percentage of the stimulation produced by 100 μM dopamine in each assay. Normalized dose-response curves were analyzed by nonlinear regression with an algorithm for sigmoid curves in the curve-fitting program Prism (Graphpad Inc., San Diego, Calif.). In all cases, analysis of the residuals indicated an excellent fit with r values greater than 0.99. For each curve, the program provided point estimates of both the EC₅₀ and the maximal stimulation. For desensitization studies, cAMP levels also were expressed initially as picomoles per minute, and then converted to percent dopamine-induced desensitization (dopamine=100%) in each assay. These values then were averaged to obtain desensitization levels for all drugs studied. Desensitization data were analyzed by one-way analysis of variance, followed by Dunnett's test. For competition binding studies, the raw data (expressed in dpm) were analyzed by nonlinear regression with a sigmoid dose-response model in Prism. The software generated estimates of both the IC₅₀ and the n_{H}. The IC₅₀ was converted to an apparent K_{0.5} with the Cheng-Prusoff equation for bimolecular competitive interactions. For saturation studies, the raw data (expressed in dpm) were analyzed by nonlinear regression with a one-site rectangular hyperbola model in Prism. The software generated estimates of both the K_{D} and Bₘₐₓ for each curve. Bₘₐₓ estimates were transformed to fmol per milligram of protein, and then converted to percent of control Bₘₐₓ. These values were analyzed by one-way analysis of variance, followed by Dunnett's test.

### Method Example 12

### Human Clinical Trial for Schizotypal Personality Disorder Entry Criteria & Inclusion Criteria

All patients and controls are medically and neurologically healthy, without current abuse of illicit substances or alcohol or a past history of substance dependence, and at least two weeks medication free of psychotropic or any systemic medications, prescription or non-prescription. Patients enter the program off all medications; or alternatively >99% medication free of entry. Patients are withdrawn from psychotropic medications if they are clearly clinically ineffective according to both the treating clinician and patient and patients are not withdrawn from neuroleptic medication. Subjects include both men and women between 18 and 60 years of age. Schizotypal personality disordered patients meet requisite DSM-IV criteria for SPD. Patients may have met criteria for major depressive disorder in the past, but not currently. It is appreciated that a history of depression may be a concomitant of schizotypal and other personality disorders and a past history of depression has not been found to affect the findings to date.

### Exclusion Criteria

Patients do not meet current or lifetime DSM-IV or RDC criteria for schizophrenia or any schizophrenia related psychotic disorder or for bipolar disorder. Other Axis I disorders are transient and preceded by the personality disorder diagnosis primarily responsible for ongoing functional impairment. Patients with neurologic complications, physical illness, low IQ, and poor visual activity are excluded.

Controls are screened for a personal history of Axis I and II disorders and family history of psychiatric disorders. Demographic characteristics are obtained and subsequently are selected for similarity to patients on the basis of parental SES.

Clinical Assessment & Diagnostic Assessment The Structured Clinical Interview for DSM-IV (SCID-I/P) is utilized to evaluate Axis I diagnoses (First et al., 1996). The Schedule for Interviewing DSM-IV Personality Disorders-IV (SIDP-IV) is utilized to evaluate criteria for DSM-IV personality disorders on the basis of one or two Master's level psychologists interviewing the patient and a third interviewing an informant close to the patient. This instrument, which has evolved over changes in the DSM, generally has a reliability of K=0.73 for SPD with a range of 0.68-0.84 for each individual SPD criterion. It is understood that the biologic studies discriminating SPD from comparison groups using this instrument may support its validity.

### Medical Evaluation Procedures

All patients and controls receive a comprehensive medical evaluation prior to their participation in any studies which includes a medical history and physical exam, complete blood count, blood chemistry (SMA-18), VDRL, thyroid function tests, routine urinalysis, urine toxicology screen, breathalyzer, EKG, ESR and a chest x-ray. Women receive a pregnancy test. Patients are excluded for presence or positive history of severe medical or neurological illness or any cardiovascular disease.

### Exclusion Criteria for Substance Abuse

All patients are screened for alcohol and drug/use/dependence using the SCID-P interview by one or two reliable raters. Patients who meet criteria for past dependence or recent abuse are excluded from the study.

### Cognitive Battery

The cognitive battery includes measures of attention including a standard visual and auditory continuous performance task: tests of working memory including the modified AX version of the CPT (AX-CPT) (Braver & Cohen, Prog. Brain Res. 121:327-49 (1999)), the N-back task (Callicott et al., Cereb. Cortex 10:1078-92 (1998); Callicott et al., Neuropsycopharmacology 18:186-96 (2000)), the DOT test of visual spatial working memory (Kirrane et al., Neuropsycopharmacology 22:14-18 (2000)) and the Paced Auditory Serial Addition Test which measures verbal working memory (Diehr et al., Assessment 5:375-87 (1998)).

Illustrative Protocol for dihydrexidine (6a) Patients are studied in a protocol room with monitoring from nursing staff of possible side-effects and vital signs every 15 minutes. Dihydrexidine or placebo is administered at 10:00AM on two distinct protocol days, separated by at least an intervening day. Dihydrexidine is administered in a dose 0.2 mg/kg (but no greater than 20 mg) administered subcutaneously. Cognitive testing is administered starting at 1:00PM for a duration of approximately an hour to an hour and a half, in which time the testing is completed on both protocol days. 15 SPD and 15 normal control subjects are entered into these protocols. Subjects are randomized, stratified within group, to a placebo first or active first condition. In addition to cognitive testing clinical assessment of symptoms are obtained using the PANSS, CGI, SPQ, Beck depression, and Spielberger Anxiety Ratings.

Patients are medication-free for at least two weeks (six weeks for fluoxetine) and refrain from smoking cigarettes past midnight the night before and throughout the days of the cognitive testing.

### Data Analytic Plan

Differences between healthy controls and SPD subjects on the cognitive outcome variables are measured by multi-variant analysis comparing placebo and drug day in both groups. Correlation on analysis with other clinical variables such as number of schizotypal criteria or D₁, receptor binding is performed with appropriate correlational analysis, either Pearson or Spearman, depending on the distributions of the data.

### Power Analysis

Effect sizes in the large range are observed in the initial pergolide trial, so that adequate power for this sample size to detect large effect size would be available in the pilot sample.

### Method Example 13

### Using fMRI to Investigate the Brain Changes Induced by a Cognitive Enhancer in Patients with Schizophrenia

This method assesses whether addition of a cognitive enhancing medication to current antipsychotic therapy may improve functionality of networks necessary in working memory and internal concept generation. Cognitive impairments may be cardinal features of schizophrenia and predictors of poor vocational and social outcome. Imaging studies with verbal fluency tasks (VFT) suggest that in schizophrenia, the combination of a failure to deactivate the left temporal lobe and a hypoactive frontal lobe reflects a functional disconnectivity between the left prefrontal cortex and temporal lobe, or an abnormal cingulate gyrus modulates such fronto-temporal connectivity.

Brain activity in 6 subjects on stable atypical antipsychotics performing a VF is serially measured, using BOLD fMRI. Measurements are made at baseline and again after groups are randomized to receive 12 weeks of donepezil (an acetylcholinesterase inhibitor) and placebo in a blind cross-over design. Donepezil addition provided a functional normalization with an increase in leftfrontal lobe and cingulate activity when compared to placebo and from baseline scans. This study provides support for the cingulate's role in modulating cognition and neuronal connectivity in schizophrenia.

### Method Example 14

### Human Clinical Trial for Regional Brain Activity (Blood Flow and Task-Specific Activation) in Patients with Schizophrenia

This method assesses whether a single dose, illustratively 20 mg subcutaneous (sc) of 6a, when compared to a saline control injection, (a) produces measurable increases in resting blood flow in the prefrontal cortex of patients with schizophrenia (as measured by contrast injection perfusion fMRI), (b) results in increased neural activity in regions involved in working memory (as measured by BOLD fMRI), (c) is tolerated with few side effects and/or (d) demonstrates a potential to improve cognitive performance.

This method includes a within subject cross-over design in 20 adults (18-65 yrs of age) with SCID diagnosed schizophrenia. Subjects are outpatients taking stable doses of antipsychotic medications, who have a moderate level of remaining negative symptoms. During a screening visit subjects are consented, rated, and receive training and practice on several computer administered neuropsychological tests. Subjects are admitted on the evening prior to testing. The following morning at 8 am they are taken to a 3T MRI scanner, with IV's, s.c and hep locks in place. They are scanned with a morning resting blood flow scan, followed by a BOLD fMRI scan during the n-back working memory task. They then receive 20 mg of a D₁ receptor agonist described herein, such as dihydrexidine 6a, or placebo, sc over 15 minutes. Over the next 45 minutes they have intermittent MRI scans of perfusion and BOLD activity during the working memory task. Response data and serum levels are also be collected. Subjects are then be returned to the hospital for observation. A repeat MRI scan is performed at 6 pm, without any infusions. The following morning they have a repeat of the Day 1 schedule, and receive either a D₁ receptor agonist described herein or placebo, whichever they did not receive on Day 1. Subjects are discharged from the hospital after the 6 pm scan on Day 2. Follow-up safety interviews are conducted at 1 week, 1 month, and 3 months post-discharge.

Inclusion Criteria include subjects with DSM-IV criteria for schizophrenia determined by the Structured Clinical Interview for DSM-IV (SCID) and with some symptoms despite treatment as defined by: PANS score >50 but less then 90, and PANS negative score of at least 4. Patients are between the ages of 18 and 65 of either gender. Patients are on stable doses of antipsychotic medications for at least 2 weeks. Patients are free of the following psychotropic medications: tricyclic antidepressants, phenothiazines, thiothixenes, clozapine, anticholinergics or stimulants for at least two weeks. Concurrent Axis II diagnoses are allowed except for Mental Retardation.

Exclusion Criteria include a past history of epilepsy or seizure disorder, mass brain lesions, metal in the skull, or a history of major head trauma; subjects who demonstrate recent (2 week) acute exacerbation of their psychosis or with catatonic subtype; subjects diagnosed with schizoaffective disorder according to the DSM-IV; subjects diagnosed with Substance Dependence (DSM-IV) and current Major Depressive Disorder (Calgary depression rating scale >9), subjects with history of clinically significant cardiovascular or cerebrovascular diseases, uncontrollable blood pressure, or abnormal ECG; subjects with renal or hepatic dysfunction; pregnant women or nursing mothers; smokers with greater than 2 packs per day use; subjects with claustrophobia or who have previously had problems with MRI scanning; and subjects with allergies to injectable contrast agents.

### Primary Study Endpoint(s)

Prefrontal Cortex Blood Flow. Resting prefrontal cortex blood flow is measured using the perfusion fMRI technique at baseline and intermittently over the hour following administration of a D₁ receptor agonist described herein, such as 20 mg of sc of 6a, or placebo, expressed as absolute data, as well as change from the morning baseline (expressed as a percent). Within day as well as between day comparisons are made to test for potentially increased rCBF with the D₁ receptor agonist.

Blood flow changes. Use echoplanar BOLD-FMRI on a specially modified 3.0 T MRI scanner to measure relative regional cerebral blood flow (rCBF) during a working memory task (the n-back).

### Secondary Study Endpoints

In order to characterize the effects of the D₁ receptor agonist in schizophrenic patients, assess reaction time and error rates on the n-back, symptom checklists of side effects and BPRS and PANS scores.

### Method Example 15

### Binding and Activity of Dihydrexidine at Dopamine receptors

| | | | Cloned Receptors (nM) | | | | |
|---|---|---|---|---|---|---|---|
| | Rat Striatum | | D_{1A} | D_{2L} | D₃ | D₄ | D₅ |
| | (nM) | | C-6 | C-6 | C-6 | CHO | HEK |
| Drug | D₁-like | D₂-like | (monkey) | (rat) | (rat) | (rat) | (human) |
| SCH 23390 | 0.69 | — | 0.32 | — | — | — | 1.0 |
| chlorpromazine | — | 1.19 | — | 0.74 | 0.9 | 20 | — |
| dihydrexidine | 5.5 | 24.4 | 2.2 | 183 | 18 | 13 | 16 |

Dihydrexidine was screened for activity at 40 binding sites (other than the D₁ site) and been found to be inactive (IC₅₀>10 μM) at all except D₂ dopamine receptors IC₅₀=130 nM) and alpha₂ adrenergic receptors (IC₅₀=ca. 230 nM). Aside from the D₁ site, dihydrexidine appears to stimulate only postsynaptic D₂ dopamine receptors. Dihydrexidine is as efficacious and is approximately 70 times more potent than dopamine in the stimulation of adenylate cyclase. This effect is blocked by the D₁ antagonist SCH 23390, but not by D₂5-HT₂, muscarinic, or alpha- or beta-adrenergic receptor antagonists. Dihydrexidine shows full efficacy in stimulating adenylate cyclase in rat, monkey, and human brain tissue. Dihydrexidine is inactive in releasing dopamine or in blocking its reuptake.

### Effects on Cognitive Behavior in Monkeys

As in Parkinson patients, primates with lesions of dopaminergic neurons exhibit difficulty in performing procedural cognitive tasks. Cognitive deficits have been reported in monkeys depleted of dopamine in the prefrontal cortex, and in asymptomatic MPTPtreated primates. Local injection of D₁ antagonists into the prefrontal cortex of monkeys induced errors and increased latency in performance of a task requiring memory guided saccades suggesting a significant role for the D₁, receptor in mnemonic, predictive function of the primate prefrontal cortex. Consistent with this interpretation are the observations of Arnsten et al. Administration of the partial D₁ agonist SKF 38393 improved spatial working memory in aged and reserpine-treated monkeys; the full D₁ agonist dihydrexidine produced improvements in young, intact monkeys. Dihydrexidine has recently been found to improve cognitive deficits in monkeys produced by chronic low dose MPTP treatment.

### Method Example 16

### Binding and activity of dinapsoline at dopamine receptors

| | | | Cloned Receptors (nM) | | | | |
|---|---|---|---|---|---|---|---|
| | Rat Striatum | | D_{1A} | D_{2L} | D₃ | D₄ | D₅ |
| | (nM) | | C-6 | C-6 | C-6 | CHO | HEK |
| Drug | D₁-like | D₂-like | (monkey) | (rat) | (rat) | (rat) | (human) |
| SCH 23390 | 0.69 | — | 0.32 | — | — | — | 1.0 |
| chlorpromazine | — | 1.19 | — | 0.74 | 0.9 | 20 | — |
| dinapsoline | 5.93 | 31.3 | 6.1 | 59 | 10 | 60 | 5.0 |
| SKF 38393 | 20 | — | 8.6 | — | — | — | 80 |
| quinpirole | >5000 | 28.8 | — | 221 | 4.5 | — | — |

Dinapsoline was as effective as dopamine in activating adenylate cyclase in rat brain striatum. In addition, dinapsoline was as effective as dopamine even when receptor reserve is reduced, indicating equal intrinsic activity.

Dinapsoline also displayed full agonist activity in stimulating adenylate cyclase (AC) at the cloned human D₁-like receptors. Dinapsoline is equally efficacious and more potent at both the D₁ and D₅ receptors when compared to dopamine. The data for several experiments are summarized in the following table, indicating that dinapsoline does not functionally discriminate between the D₁ and D₅ receptors for stimulating AC:

Dinapsoline potently activates hD₁ and hD₅ receptors

EC₅₀ (nM) ± SEM

| Test Ligand | D₁ | D₅ |
|---|---|---|
| dopamine | 486 ± 157 | 114 ± 186 |
| dinapsoline | 28 ± 9 | 10 ± 2 |

Studies completed in HEK cells represent at least three separate experiments (expressed as mean±SEM).

The interaction of dinapsoline with D₂-like receptors coupled to a number of different signaling systems has been studied. The most widely used endpoint, adenylate cyclase (AC), is stimulated by D₁-like receptors, yet D₂-like receptors inhibit cAMP synthesis. Full agonist activity is gauged by comparison of the activity of a test ligand to the activity of dopamine or the prototypical D₂ agonist quinpirole.

The ability of dinapsoline to inhibit forskolin (FSK)-stimulated AC activity through D_{2L} and D₄ receptors expressed in CHO cells was studied.

Dinapsoline inhibits AC to the same extent as the prototypical D₂ agonist quinpirole. This result is indicative of full agonist activity at D_{2L} receptors coupled to cAMP synthesis. The following table summarizes the effects of dinapsoline at both D_{2L} and D₄ receptors, indicating that dinapsoline is a full agonist for the inhibition of cAMP synthesis at both D_{2L} and D₄ receptors expressed in CHO cells.

Dinapsoline potently activates D_{2L} and D₄ receptors

EC₅₀ (nM) ± SEM

| Test Ligand | D_{2L} | D₄ |
|---|---|---|
| dopamine | — | 1752 ± 682 |
| dinapsoline | 81 ± 21 | 60 ± 18 |
| quinpirole | 3 ± 1 | — |

At least three separate experiments were performed (expressed as mean±SEM).

1. A pharmaceutical composition comprising a dopamine D₁ receptor agonist; a dopamine D₂ receptor antagonist; and a pharmaceutically acceptable carrier, diluent, excipient, or combination thereof, wherein the amount of the dopamine D₁ receptor agonist and the amount of the dopamine D₂ receptor antagonist are each effective for treating a patient at risk of developing or having a neurological, psychotic, or psychiatric disorder.

2. The pharmaceutical composition of claim 1 wherein the dopamine D1 receptor agonist is a compound selected from the group consisting of hexahydrobenzophenanthridines, hexahydrothienophenanthridines, phenylbenzazepines, chromenoisoquinolines, naphthoisoquinolines, analogs and derivatives thereof, pharmaceutically acceptable salts thereof, and combinations thereof.

3. The pharmaceutical composition of claim 1 wherein the neurological, psychotic, or psychiatric disorder is selected from the group consisting of schizophrenia, schizophreniform disorders, schizoaffective disorders, cognitive disorders, memory disorders, autism, Alzheimer's disease, dementia, bipolar disorder, depression in combination with psychotic episodes, and other disorders that include a psychosis.

4. The pharmaceutical composition of claim 1 wherein the dopamine D₁ receptor agonist is a full agonist.

5. The pharmaceutical composition of claim 1 wherein the dopamine D₁ receptor agonist is selective for a dopamine D₁ receptor subtype.

6.-8. (canceled)

9. The pharmaceutical composition of claim 1 wherein the dopamine D₂ receptor antagonist does not exhibit significant binding at the dopamine D₁ receptor.

10.-12. (canceled)

13. The pharmaceutical composition of claim 1 wherein the dopamine receptor agonist is a compound selected from the group of formulae consisting of (a) 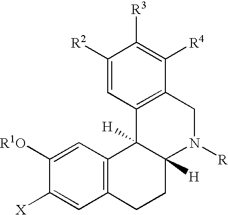
wherein
R is hydrogen or C₁-C₄ alkyl;
R¹ is hydrogen, acyl, benzoyl, pivaloyl, an optionally substituted phenyl protecting group;
X is hydrogen, fluoro, chloro, bromo, iodo; or X is a group having the formula —OR⁵ wherein R⁵ is hydrogen, C₁-C₄ alkyl, acyl, benzoyl, pivaloyl, an optionally substituted phenyl protecting group; or the groups R¹ and R⁵ are taken together to form a divalent radical having the formula —CH₂— or —(CH₂)₂—; and
R², R³, and R⁴ are each independently selected from the group consisting of hydrogen, C₁-C₄ alkyl, phenyl, fluoro, chloro, bromo, iodo, and a group —OR⁶ wherein R⁶ is hydrogen, acyl, benzoyl, pivaloyl, or an optionally substituted phenyl protecting group;
or a pharmaceutically acceptable salt thereof; 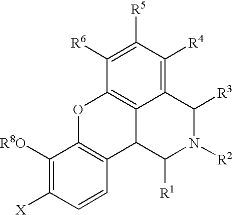
wherein
R¹, R², and R³ are each independently selected from the group consisting of hydrogen, C₁-₄ alkyl and C₂-C₄ alkenyl:
R⁴, R⁵, and R⁶ are each independently selected from the group consisting of hydrogen, C₁-C₄ alky, phenyl, halo, and a group having the formula —OR, where R is hydrogen, acyl, benzoyl, pivaloyl, or an optionally substituted phenyl protecting group,
R⁸ is hydrogen, C₁-C₄ alkyl, acyl, or an optionally substituted phenyl protecting group;
X is hydrogen or halo; or X is a group having the formula —OR⁹, where R⁹ is hydrogen, C₁-C₄ alkyl, acyl, or an optionally substituted phenyl protecting group; or when X is a group having the formula —OR⁹, R⁸ and R⁹ are taken together to form a divalent group having the formula —CH₂—;
or a pharmaceutically acceptable salt thereof; and
(c) 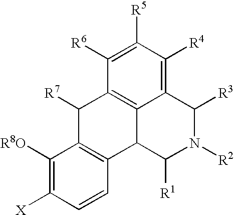
wherein
R¹, R², and R³ are each independently selected from the group consisting of hydrogen, C₁-C₄ alkyl and C₂-₄-alkenyl;
R⁴, R⁵, and R⁶ are each independently selected from the group consisting of hydrogen, C₁-C₄ alkyl, phenyl, halogen, and a group having the formula —OR, where R is hydrogen, acyl, benzoyl, pivaloyl, or an optionally substituted phenyl protecting group;
R⁷ is selected from the group consisting of hydrogen, hydroxy, C₁-C₄ alkyl C₂-C₄ alkenyl, C₁-C₄ alkoxy, and C₁-C₄ alkylthiol
R⁸ is hydrogen, C₁-C₄ alkyl, acyl, or an optionally substituted phenyl protecting group; and
X is hydrogen, fluoro, chloro, bromo, or iodo; or X is a group having the formula —OR⁹, where R⁹ is hydrogen, C₁-C₄ alkyl, acyl, or an optionally substituted phenyl protecting group; or when X is a group having the formula —OR⁹, R⁸ and R⁹ are taken together to form a divalent group having the formula —CH₂—;
and pharmaceutically acceptable salts thereof.

14. (canceled)

15. The pharmaceutical composition of claim 13 wherein the dopamine receptor agonist is a compound of formula (a), and at least one of the groups R², R³, and R⁴ is other than hydrogen.

16. The pharmaceutical composition of claim 13 wherein the dop amine receptor agonist is a compound of formula (a), and R is hydrogen or methyl; R¹ is hydrogen; X is hydrogen, bromo, or —OR², and R² is hydrogen.

17.-28. (canceled)

29. The pharmaceutical composition of claim 13 wherein the dopamine receptor agonist has a half-life in the range from about 30 minutes to about 3 hours.

30.-32. (canceled)

33. The pharmaceutical composition of claim 13 wherein the dopamine receptor agonist is a compound of formula (b), and at least one of the groups R⁴, R⁵, and R⁶ is other than hydrogen.

34.-36. (canceled)

37. The pharmaceutical composition of claim 13 wherein the dopamine receptor agonist is a compound of formula (c), and at least one of the groups R⁴, R⁵, and R⁶ is other than hydrogen.

38. The pharmaceutical composition of claim 1 wherein the dopamine D₂ receptor antagonist is an antipsychotic agent.

39. The pharmaceutical composition of claim 1 wherein the dopamine D₂ receptor antagonist is an atypical antipsychotic agent.

40. The pharmaceutical composition of claim 1 further comprising one or more cholinergic agents, cholinergic agonists, acetylcholine mimetics, acetylcholine esterase inhibitors, or combinations thereof.

41. A method for treating a patient at risk of developing and/or having a neurological, psychotic, and/or psychiatric disorder, said method comprising the step of administering to the patient an effective amount of a composition according to claim 1.

42. A method for treating a patient at risk of developing and/or having a neurological, psychotic, and/or psychiatric disorder, said method comprising the steps of:
administering to the patient an effective amount of a full dopamine D₁ receptor agonist, where the agonist is a compound selected from the group consisting of hexahydrobenzophenanthridines, hexahydrothienophenanthridines, phenylbenzodiazepines, chromenoisoquinolines, naphthoisoquinolines, pharmaceutically acceptable salts thereof, and combinations thereof; and
administering to the patient an effective amount of a dopamine D₂ receptor antagonist;
where the agonist and the antagonist are administered contemporaneously.

43. The method of claim 41 wherein the agonist and the antagonist are administered simultaneously.

44. The method of claim 41 wherein the agonist and the antagonist are administered in a unitary dosage form.

45.-62. (canceled)
